(19) United States
(12) Patent Application Publication
(10) Pub. No.: US 2007/0160976 A1
(43) Pub. Date: Jul. 12, 2007

(54) Novel cellular function regulating agent produced by a chondrocyte capable of hypertrophication

(75) Inventor: Hiroyuki Okihana, Itabashi-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: PENTAX Corporation, Itabashi-ku (JP)

(21) Appl. No.: 11/643,621

(22) Filed: Dec. 20, 2006

(30) Foreign Application Priority Data
Dec. 20, 2005 (JP) 2005-367174
Dec. 8, 2006 (JP) 2006-332687

(51) Int. Cl. C12Q 1/00; C12N 5/08; C12N 5/06

(52) U.S. Cl. 435/4; 435/325; 435/366; 435/353; 435/354

(57) ABSTRACT

The present invention provides an agent obtainable by culturing a chondrocyte capable of hypertrophication in a differentiation agent producing medium, wherein the differentiation agent producing medium comprises at least one conventional osteoblast differentiation component selected from the group consisting of glucocorticoid, β-glycerophosphate and ascorbic acid. The differentiation agent producing medium may comprise all of glucocorticoid, β-glycerophosphate and ascorbic acid.

### CROSS-REFERENCE TO RELATED APPLICATIONS

The present application claims priority to Japanese Patent Application No. 2005-367174, filed on Dec. 20, 2005 and Japanese Patent Application No. 2006-332687 filed on Dec. 8, 2006, which are herein incorporated in their entirety.

### TECHNICAL FIELD

The present invention relates to a cellular function regulating agent produced by a chondrocyte capable of hypertrophication and a production method and methods of use thereof.

### BACKGROUND ART

Osteogenesis is a preferred method to treat diseases associated with decreasing osteogenesis, damage of the bone, or bone deficits. When a bone tissue sustains damage such as fracture or abscission due to bone tumor, bone generating cells, known as osteoblasts, proliferate and differentiate to regenerate bone, and thereby cure bone fracture or deficits. In the case of mild damage, immobilization of the bone at the affected area allows osteoblasts to be activated, and thereby the adjacent area is repaired. When osteoblasts cannot be effectively activated in circumstances such as complex fracture, large damages of the ostectomy, or damage in combination with osteomyelitis, autologous bone transplantation is generally considered as a standard treatment for such damage or deficits. When the damaged region is too large to repair with autologous bone, artificial bone may be used in partial combination with autologous bone. However, in humans, sources of autologous bone are limited and the amount available for collection is limited. In addition, a disadvantage arises when an additional operation is required to collect bone of limited availability. Moreover, supplying autologous bone is accompanied by high costs and pain to the donor. Furthermore, the use of autologous bone causes a new deficit to the region where the normal autologous bone is originated from.

Therefore, surgical treatments using an artificial bone implant and other bone repairing materials have been introduced. It is also possible to repair a regional defect of an organic tissue (e.g., bone) by implanting reparative organic tissue such as a bone repairing material when such defective region is derived from trauma and resection of bone tumors. Hydroxyapatite (HAP) and tricalcium phosphate (TCP) are known mainly as bone repairing materials.

However, compared to autologous bone, conventional artificial bone implants and bone repair materials also have disadvantages such as poor osteogenic ability, difficulties in generating bone, low rigidity and fragility. Therefore, after these surgical procedures, the prognosis for such procedures is often poor and multiple operations are often needed. Although the rate of using artificial bone has increased, for the above reasons, it remains at about 30% while autologous bone is used at the remaining 60-70% instance.

In the United States, allogeneic bone is often used. On the other hand, in Japan, the use of cavaderic tissues is unfamiliar, and thus cavaderic tissues are not used so often. Although Bone Banks are an alternative way of providing autologous bone, so far, the stock is insufficient.

In order to improve the above-stated disadvantages of conventional artificial bone, attempts have been made to utilize regenerative medicine using the regenerative ability of cells, and to apply treatments for fractures and bone deficits. These attempts have also been applied to increase the rate of repairing bone deficits after surgical procedures. Stem cells derived from bone marrow are generally used in such regenerative medicine. It has been proposed to use an implant of tissues of biological organisms, including cultured bone and the like, which is produced by incubating bone marrow stem cells and differentiated osteoblasts derived from a patient with a bone repairing material. Bone repairing materials, which include many bone marrow mesenchymal stem cells and differentiated osteoblasts, are implanted into a defective region of bone, wherein the bone marrow mesenchymal stem cells and differentiated osteoblasts are proliferated on a bone repairing material as a scaffold. The aforementioned disadvantages of artificial bone can be compensated and improved by reducing the period of osteogenesis, in comparison to methods of implanting a bone repairing material only.

In order to differentiate mesenchymal stem cells derived from bone marrow to osteoblasts in conventional methods of the regeneration, Maniatopoulos et al. has described a method using three compounds consisting of dexamethasone, β-glycerophosphate and ascorbic acid, and a method using modified working concentrations has also been described. However, these methods are artificial, not natural. There are undifferentiated stem cells among those treated by these three compounds. As a result, there is an anxiety for the property and function of a differentiated osteoblast.

Therefore, there is a need to provide safe, low-cost and stable osteoblasts for treating diseases associated with the decrease of osteogenesis, damage of the bone or bone deficits.

It is believed that BMP (Bone Morphogenetic Protein)-2, BMP-4, and BMP-7 play important role in osteogenesis by inducing osteoblasts. The BMP-2, BMP-4, and BMP-7 are believed to induce to osteoblasts. There are many family members of the BMP family. However, other than BMP-2, BMP-4 and BMP-7 homologs, the family members are obtained based on the sequence of previously identified BMP-2 and lack references to their function, which do not always have potentials for inducing the differentiation of osteoblasts. It is reported that BMP-2, BMP-4 and BMP-7 differentiate into osteoblast effectively in mice and rats, but the efficiency is only a thousandth of that in human (Wozney, J. M. et al.: Novel Regulators of Bone Formation: Molecular Clones and Activities. Science, 242: 1528-1534, 1988, Wuerzler K K et al.: Radiation-Induced Impairment of Bone Healing Can Be overcome by Recombinant Human Bone Morphogenetic Protein-2. J. Craniofacial Surg., 9: 131-137, 1998, Govender S et al.: Recombinant Human Bone Morphogenetic Protein-2 for treatment of Open Tibial Fractures. J. Bone Joint Surg., 84A: 2123-2134, 2002, Johnsson R et al.: Randomized Radiostereometric Study Comparing Osteogenic Protein-1 (BMP-7) and Autograft Bone in Human Noninstrumented Posterolateral Lumber Fusion. Spine, 27: 2654-2661, 2002.).

I observed that osteogenesis due to intracartilaginous ossification is induced by implanting BMP into heterotopias. Wozney et al. who cloned BMP used the term “cartilage-inducing activity” upon measuring the activity of BMP (Wozney, J. M. et al.: Novel Regulators of Bone Formation: Molecular Clones and Activities. Science, 242: 1528-1534, 1988.). I reported that osteogenesis is not directly induced by BMP-2, BMP-4, and BMP-7, but by an agent producing a chondrocyte capable of hypertrophication to differentiate osteoblasts, wherein the chondrocyte capable of hypertrophication is induced by BMP-2, BMP-4, and BMP-7 (Okihana, H.: seichonankotsu no seisansuru honekeiseuinshi [an osteogenesic agent producing growth cartilage], igaku no ayumi [Journal of Clinical and Experimental Medicine], 165: 419, 1993, Okihana, H. & Shimomura, Y: Osteogenic Activity of Growth Cartilage Examined by Implanting Decalcified and Devitalized Ribs and Costal Cartilage Zone, and Living Growth Cartilage Cells. Bone, 13: 387-393, 1992). This unknown agent, which is a peptide or derived from a biological organism, has a molecular weight of 50,000 or more and directly affects the induction, chemotaxis and activation of osteoblasts.

Japanese Laid-Open Publication No. 2004-305259 disclosed a production method of biological tissue prosthesis. The production method comprises of adhering stem cells at biological tissue prosthesis, inducing the adhered stem cells to differentiation, thereby making the effect of formation of a biological tissue using biological tissue prosthesis as a scaffold, and treating to devitalize the formed tissue and cells. Japanese Laid-Open Publication No. 2004-305259 does not describe a cell-regulating agent produced by a chondrocyte that is capable of hypertrophication and inducing the differentiation of undifferentiated cells into osteoblasts.

Japanese Laid-Open Publication No. 2004-305260 disclosed a production method of biological tissue prosthesis. The production method comprises of adhering stem cells at biological tissue prosthesis, inducing the adhered stem cells to the differentiation, thereby making the effect of formation of biological tissue using biological tissue prosthesis as a scaffold; and treating to dissolute the formed tissue and cells, wherein the step of treating comprises freezing the biological tissue prosthesis and drying thereof. Japanese Laid-Open Publication No. 2004-305260 does not describe a cell-regulating agent produced by a chondrocyte that is capable of hypertrophication and inducing the differentiation of undifferentiated cells into osteoblasts.

Japanese Laid-Open Publication No. 2004-49142 disclosed method for producing cultured bone. The method for producing the cultured bone comprises a primary culturing step for obtaining a mesenchymal stem cell by culturing a bone marrow cell collected from a patient in a prescribed culture medium, a secondary culturing step for differentiating the cultured mesenchymal stem cell to an osteoblast by culturing the cultured mesenchymal stem cell in a prescribed bone-forming culture medium, a recovery step for recovering the differentiated osteoblasts and a produced bone substrate and a mixing step for mixing the recovered osteoblast and bone substrate with bone prosthetic material granules. Japanese Laid-Open Publication No. 2004-49142 does not describe a cell-regulating agent produced by a chondrocyte that is capable of hypertrophication and inducing the differentiation of undifferentiated cells into osteoblasts.

Japanese Laid-Open Publication No. 2005-205074 disclosed a method for manufacturing the cultured bone by making a bone filler material carrying mesenchymal stem cells obtained by culturing the cells sampled from a patient, culturing the mesenchymal stem cells carried by the bone filler material, and differentiating to osteoblasts. The publication also disclosed a method for manufacturing the cultured bone in which the osteoblasts are carried by the bone filler material, after culturing the mesenchymal stem cells obtained from the cells sampled from the patient and differentiating to the osteoblasts. In this method, platelet-rich plasma needs to be added to the culture liquid for culturing the cells sampled from the patient, the culture liquid for culturing the mesenchymal stem cells, or the culture liquid after differentiating to the osteoblasts. Japanese Laid-Open Publication No. 2005-205074 does not describe a cell-regulating agent produced by a chondrocyte that is capable of hypertrophication and inducing the differentiation of undifferentiated cells into osteoblasts.

Japanese National Phase PCT Laid-Open Publication No. 2003-531604 disclosed a method for isolating a mesenchymal stem cell from the human tissue after birth and including the human prepuce tissue after birth, as well as a method for differentiating the isolated mesenchymal stem cells to various cell lineages including osteogenesis, adipogenesis, and a cartilage formation lineage and the like. Japanese Laid-Open Publication No. 2003-531604 does not describe a cell-regulating agent produced by a chondrocyte that is capable of hypertrophication and inducing the differentiation of undifferentiated cells into osteoblasts.

### SUMMARY OF THE INVENTION

The object of the present invention is to provide a cellular function regulating agent producing a chondrocyte capable of hypertrophication, as well as a method for the production and uses thereof. The agent is available to treat the diseases associated with the decrease of osteogenesis, damage of the bone, or bone deficits, especially bone tumors, complex fractures and the like.

The object of the present invention is to provide an agent which produces a chondrocyte capable of hypertrophication and is a novel cell-regulating agent with respect to the osteogenetic ability, safety thereof, rate of bone regeneration, strength of the regenerated bone and so on.

The object of the present invention is to provide an agent capable of inducing the differentiation of osteoblasts to a board range of cells including conventional cell lines and/or cells distinct from conventional cells.

The objects mentioned above have been partially solved in the present invention by finding that a chondrocyte capable of hypertrophication produces a cell-regulating agent capable of inducing the differentiation of the undifferentiated cell into an osteoblast, and that the agent has a potential for inducing the differentiation of osteoblasts for a wide rang of cells including conventional cell lines and/or non-conventional cells.

The present invention provides that an agent, which is a peptide or derived from a biological organism, has a molecular weight of 50,000 or more and directly affects the induction, chemotaxis, activation of osteoblast. This chondrocyte capable of hypertrophication is capable to induce osteogenesis by differentiating directly to an osteoblast, unlike the low molecular weight inducers of BMP-2, BMP-4, and BMP-7.

To achieve the objects mentioned above, the present invention provides the following:

In one aspect, the present invention provides an agent obtainable by culturing a chondrocyte capable of hypertrophication in a differentiation agent producing medium, wherein the differentiation agent producing medium comprises at least one conventional osteoblast differentiation component selected from the group consisting of glucocorticoid, β-glycerophosphate and ascorbic acid.

In one embodiment, the agent according to the present invention, is separable to a fraction of a molecular weight over 50,000, wherein the culture supernatant cultured in the differentiation agent producing medium is placed in a centrifugal filter and subjected to centrifugal ultrafiltration of 4,000×g, 4° C. for 30 minutes under conditions suitable for the separation of a macromolecular weight fraction and a low molecular weight fraction.

In another embodiment, the agent according to the present invention is capable of inducing differentiation of osteoblasts from undifferentiated cells.

In one embodiment, the undifferentiated cell employed in the present invention is a cell which is not differentiated by glucocorticoid, β-glycerophosphate and ascorbic acid.

In another embodiment, the agent according to the present invention is capable of increasing the value of alkaline phosphatase (ALP) activity of a C3H10T1/2 cell, which is exposed to the agent in Eagle's basal medium, to more than about one time of that of the cell cultured in Eagle's basal medium without the agent, wherein the alkaline phosphatase activity is determined by following steps:

A) determining two absorbances at 405 nm, wherein to one absorbance sample of 100 μl with or without the agent, 50 μl of 4 mg/ml p-nitrophenyl phosphate and 50 μl of alkali buffer (pH 10.3) are added respectively, reacted at 37° C. for 15 minutes, and 50 μl 1N NaOH is added to terminate the reaction, and to the other absorbance sample, a further 20 μl concentrated hydrochloric acid is added; and

B) calculating the difference in absorbance before and after addition of the concentrated hydrochloric acid, wherein the difference of absorbance is an indicator of the alkaline phosphatase activity.

In another embodiment, the agent according to the present invention is capable of increasing the value of alkaline phosphatase (ALP) activity of a C3H10T1/2 cell when the C3H10T1/2 cell is exposed to the agent in Eagle's basal medium, wherein the alkaline phosphatase activity is determined by the following steps:

A) determining two absorbances at 405 nm, wherein to one absorbance sample of 100 μl with or without the agent, 50 μl of 4 mg/ml p-nitrophenyl phosphate and 50 μl of alkali buffer (pH 10.3) are added respectively, reacted at 37° C. for 15 minutes, and 50 μl 1N NaOH is added to terminate the reaction, and to the other absorbance sample a further 20 μl concentrated hydrochloric acid is added; and

B) calculating the difference in absorbance before and after addition of the concentrated hydrochloric acid, wherein the difference in absorbance is an indicator of the alkaline phosphatase activity.

In another embodiment, the agent according to the present invention is capable of enhancing expression of a specific substance for osteoblasts selected from the group consisting of type I collagen, bone proteoglycan, alkaline phosphatase, osteocalcin, matrix Gla protein, osteoglycin, osteopontin, bone sialic acid protein, osteonectin and pleiotrophin.

In one embodiment, the agent according to the present invention has a property selected from the group consisting of preventing induction of the differentiation of undifferentiated cells into osteoblasts and preventing induction of the alkaline phosphatase activity in undifferentiated cells by heating for 3 minutes in boiling water.

In another embodiment, the agent according to the present invention is prevented to induce differentiation of undifferentiated cells into osteoblasts by heating for 3 minutes in boiling water.

In another embodiment, the agent according to the present invention is prevented to induce the alkaline phosphatase activity by heating for 3 minutes in boiling water.

In one embodiment, the chondrocyte capable of hypertrophication employed in the present invention is derived from a mammal.

In another embodiment, the mammal employed in the present invention is a human, a mouse, a rat, or a rabbit.

In one embodiment, the chondrocyte capable of hypertrophication employed in the present invention is a cell sampled from the region selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture, and the cartilaginous part of a bone proliferation phase.

In another embodiment, the chondrocyte capable of hypertrophication employed in the present invention is a cell capable of hypertrophication induced by differentiation.

In another embodiment, the chondrocyte capable of hypertrophication employed in the present invention expresses at least one marker selected from the group consisting of type X collagen, alkaline phosphatase, osteonectin, type II collagen, cartilage proteoglycan or components thereof, hyaluronic acid, type IX collagen, type XI collagen, or chondromodulin.

In one embodiment, the ability of hypertrophication of the chondrocyte capable of hypertrophication employed in the present invention is discriminated by morphological change.

In another embodiment, the chondrocyte capable of hypertrophication employed in the present invention is determined to be capable of hypertrophication when a significant increase in size thereof is observed by preparing a pellet of the cells by centrifugation of HAM's F12 culture medium including 5×10⁵ cells, culturing the pellet for a pre-determined period, and comparing the size of the cells observed under a microscope before culture with the size after the culture thereof.

In one embodiment, the differentiation agent producing medium employed in the present invention comprises at least one conventional osteoblast differentiation component selected from the group consisting of β-glycerophosphate and ascorbic acid.

In another embodiment, the differentiation agent producing medium employed in the present invention comprises both β-glycerophosphate and ascorbic acid as the conventional osteoblast differentiation components.

In another embodiment, the differentiation agent producing medium employed in the present invention comprises all of glucocorticoid, β-glycerophosphate and ascorbic acid as the conventional osteoblast differentiation components.

In another embodiment, the differentiation agent producing medium employed in the present invention comprises Minimum Essential Medium (MEM) or HAM Medium as the basic medium component.

In another embodiment, the differentiation agent producing medium employed in the present invention comprises Minimum Essential Medium (MEM) as the basic component, and β-glycerophosphate and ascorbic acid as the conventional osteoblast differentiation components.

In another embodiment, the differentiation agent producing medium employed in the present invention comprises Minimum Essential Medium (MEM) or HAM medium as a basic medium, and all of glucocorticoid, β-glycerophosphate and ascorbic acid as the conventional osteoblast differentiation components.

In another embodiment, the differentiation agent producing medium employed in the present invention comprises all of comprises Minimum Essential Medium (MEM), glucocorticoid, β-glycerophosphate and ascorbic acid.

In a further embodiment, the differentiation agent producing medium employed in the present invention further comprises a serum component.

In one embodiment, the agent obtainable by culturing a chondrocyte capable of hypertrophication in a differentiation agent producing medium, according to the present invention, is obtained from the supernatant of the differentiation agent producing medium.

In another embodiment, the agent obtainable by culturing a chondrocyte capable of hypertrophication in a differentiation agent producing medium, according to the present invention, exists within the chondrocyte capable of hypertrophication.

In one aspect, the present invention provides a composition comprising the agent obtainable by culturing a chondrocyte capable of hypertrophication in a differentiation agent producing medium.

In one aspect, the present invention provides a composition for inducing the differentiation of osteoblasts comprising the agent obtainable by culturing a chondrocyte capable of hypertrophication in a differentiation agent producing medium.

In one aspect, the present invention provides a composition for inducing differentiation of undifferentiated cells into osteoblasts comprising the agent obtainable by culturing a chondrocyte capable of hypertrophication in a differentiation agent producing medium.

In one embodiment, the composition according to the present invention further comprises at least one conventional osteoblast differentiation component selected from the group consisting of glucocorticoid, β-glycerophosphate and ascorbic acid.

In another embodiment, the composition according to the present invention further comprises at least one conventional osteoblast differentiation component selected from the group consisting of β-glycerophosphate and ascorbic acid.

In another embodiment, the composition according to the present invention further comprises both β-glycerophosphate and ascorbic acid as the conventional osteoblast differentiation components.

In another embodiment, the differentiation agent producing medium employed in the composition according to present invention further comprises all of glucocorticoid, β-glycerophosphate and ascorbic acid as the conventional osteoblast differentiation components.

In one aspect, the present invention provides a method of producing a composition comprising an agent capable of inducing differentiation of osteoblasts, wherein the method comprises culturing a chondrocyte capable of hypertrophication in a differentiation agent producing medium, wherein the differentiation agent producing medium comprises at least one conventional osteoblast differentiation component selected from the group consisting of glucocorticoid, β-glycerophosphate and ascorbic acid.

In one embodiment, the method of producing a composition according to the present invention comprises harvesting a supernatant of the differentiation agent producing medium.

In another embodiment, the method of producing a composition according to the present invention further comprises extracting from the supernatant of the differentiation agent producing medium.

In another embodiment, the chondrocyte capable of hypertrophication employed in the method of producing a composition according to the present invention is derived from a mammal.

In another embodiment, the mammal employed in the method of producing a composition according to the present invention is a human, a mouse, a rat, or a rabbit.

In one embodiment, the chondrocyte capable of hypertrophication employed in the method of producing a composition according to the present invention is a cell sampled from the region selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture, and the cartilaginous part of a bone proliferation phase.

In another embodiment, the chondrocyte capable of hypertrophication employed in the method of producing a composition according to the present invention is a cell capable of hypertrophication induced by differentiation.

In one embodiment, the differentiation agent producing medium employed in the method of producing a composition according to the present invention comprises at least one conventional osteoblast differentiation component selected from the group consisting of β-glycerophosphate and ascorbic acid.

In another embodiment, the differentiation agent producing medium employed in the method of producing a composition according to the present invention comprises both β-glycerophosphate and ascorbic acid as the conventional osteoblast differentiation components.

In another embodiment, the differentiation agent producing medium employed in the method of producing a composition according to the present invention comprises all of glucocorticoid, β-glycerophosphate and ascorbic acid as the conventional osteoblast differentiation components.

In another embodiment, the differentiation agent producing medium employed in the method of producing a composition according to the present invention comprises Minimum Essential Medium (MEM) or HAM Medium as the basic medium component.

In a further embodiment, the differentiation agent producing medium employed in the method of producing a composition according to the present invention comprises Minimum Essential Medium (MEM) as basic component, and β-glycerophosphate and ascorbic acid as conventional osteoblast differentiation components.

In another embodiment, the differentiation agent producing medium employed in the method of producing a composition according to the present invention comprises Minimum Essential Medium (MEM) or HAM medium as a basic medium, and all of glucocorticoid, β-glycerophosphate and ascorbic acid as the conventional osteoblast differentiation components.

In another embodiment, the differentiation agent producing medium employed in the method of producing a composition according to the present invention comprises all of comprises Minimum Essential Medium (MEM), glucocorticoid, β-glycerophosphate and ascorbic acid. In one embodiment, the differentiation agent producing medium employed in the method of producing a composition according to the present invention further comprises a serum component.

In one embodiment, the agent capable of inducing differentiation employed in the method of producing a composition according to the present invention is secreted into the supernatant of the differentiation agent producing medium.

In another embodiment, the agent capable of inducing differentiation employed in the method of producing a composition according to the present invention exists within the chondrocyte capable of hypertrophication.

In one aspect, the present invention provides a method of producing an osteoblast by induction to differentiate an undifferentiated cell into an osteoblast, comprising the steps of:

A) inoculating the undifferentiated cell to a culture scaffold or a culture vessel; and

B) exposing the undifferentiated cell to the agent according to claim 1 by adding a solution including the agent according to claim 1 to the medium or by exchanging the medium for a medium including the agent, after the undifferentiated cell is stabilized.

In one embodiment, the undifferentiated cell employed in the method of producing an osteoblast according to the present invention is derived from a mammal.

In another embodiment, the mammal employed in the method of producing an osteoblast according to the present invention is a human, a mouse, a rat, or a rabbit.

In one embodiment, the chondrocyte capable of hypertrophication employed in the method of producing an osteoblast according to the present invention is a cell sampled from the region selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture, and the cartilaginous part of a bone proliferation phase.

In one embodiment, the medium culturing the undifferentiated cell employed in the method of producing an osteoblast according to the present invention is Eagle's basal medium (BME), Minimum Essential Medium (MEM), Dulbecco's Modified Eagle Medium (DMEM) or HAM medium, or a combination thereof.

In one embodiment, the undifferentiated cell employed in the method of producing an osteoblast according to the present invention is selected from the group consisting of an embryonic stem cell, an embryonic germ stem cell and a tissue stem cell.

In one embodiment, the tissue stem cell employed in the method of producing an osteoblast according to the present invention is selected from the group consisting of a mesenchymal stem cell, a hematopoietic stem cell, a vascular stem cell, a hepatic stem cell, a pancreatic (common) stem cell, and a neural stem cell.

In another embodiment, the tissue stem cell employed in the method of producing an osteoblast according to the present invention is a mesenchymal stem cell.

In a further embodiment, the mesenchymal stem cell employed in the method of producing an osteoblast according to the present invention is a stem cell derived from bone marrow.

In another embodiment, the mesenchymal stem cell employed in the method of producing an osteoblast according to the present invention is derived from adipose tissue, synovial tissue, muscular tissue, peripheral blood, placental tissue, menstrual blood, or cord blood.

In another embodiment, the undifferentiated cell employed in the method of producing an osteoblast according to the present invention is a cell selected from the group consisting of a C3H 10T1/2 cell, an ATDC5 cell, a 3T3-Swiss albino cell, a BALB/3T3 cell, and a NIH3T3 cell.

In another embodiment, the undifferentiated cell employed in the method of producing an osteoblast according to the present invention is a cell selected from the group consisting of a C3H 10T1/2 cell, a 3T3-Swiss albino cell, a BALB/3T3 cell, and a NIH 3T3 cell.

In one aspect, the present invention provides an osteoblast which is induced by contact with an agent derived from a chondrocyte capable of hypertrophication.

In one embodiment, the agent employed in the osteoblast according to the present invention is capable of increasing the value of alkaline phosphatase (ALP) activity of a C3H10T1/2 cell, which is exposed to the agent in Eagle's basal medium, to more than about one time that of the cell cultured in Eagle's basal medium without the agent, wherein the alkaline phosphatase activity is determined by the following steps:

A) determining two absorbancies at 405 nm, wherein to one absorbance sample of 100 μl with or without the agent 50 μl of 4 mg/ml p-nitrophenyl phosphate and 50 μl of alkali buffer (pH 10.3) are added respectively, reacted at 37° C. for 15 minutes, and 50 μl 1N NaOH is added to terminate the reaction, and to the other absorbance sample, a further 20 μl concentrated hydrochloric acid is added; and

B) calculating the difference in absorbance before and after addition of the concentrated hydrochloric acid, wherein the difference in absorbance is an indicator of the alkaline phosphatase activity.

In one embodiment, the agent employed in the osteoblast according to the present invention is capable of increasing the value of the alkaline phosphatase (ALP) activity of a C3H10T1/2 cell when the C3H10T1/2 cell is exposed to the agent in Eagle's basal medium, wherein the alkaline phosphatase activity is determined by the following steps:

A) determining two absorbancies at 405 nm, wherein to one absorbance sample of 100 μl with or without the agent, 50 μl of 4 mg/ml p-nitrophenyl phosphate and 50 μl of alkali buffer (pH 10.3) are added respectively, reacted at 37° C. for 15 minutes, and 50 μl 1 N NaOH is added to terminate the reaction, and to the other absorbance sample, a further 20 μl concentrated hydrochloric acid is added; and

B) calculating the difference in absorbance before and after addition of the concentrated hydrochloric acid, wherein the difference in absorbance is an indicator of the alkaline phosphatase activity.

In one embodiment, the osteoblast according to the present invention is derived from an undifferentiated cell.

In one aspect, the present invention provides a method of producing an agent capable of inducing differentiation of osteoblasts, wherein the method comprises culturing a chondrocyte capable of hypertrophication in a differentiation agent producing medium, wherein the differentiation agent producing medium comprises at least one conventional osteoblast differentiation component selected from the group consisting of glucocorticoid, β-glycerophosphate and ascorbic acid.

In one aspect, the present invention provides a composition for use in producing an agent capable of inducing the differentiation of osteoblasts, wherein the composition comprises a chondrocyte capable of hypertrophication.

In one aspect, the present invention provides a kit for producing an agent capable of inducing differentiation of osteoblasts comprising:

A) a chondrocyte capable of hypertrophication; and

B) at least one conventional osteoblast differentiation component selected from the group consisting of glucocorticoid, β-glycerophosphate and ascorbic acid.

In one aspect, the present invention provides a composite material for producing an agent capable of inducing differentiation of osteoblasts comprising:

A) a chondrocyte capable of hypertrophication; and

B) a scaffold.

In one embodiment, the scaffold employed in the composite material according to the present invention comprises a material selected from the group consisting of calcium phosphate, calcium carbonate, alumina, zirconia, apatite-wollastonite deposited glass, gelatin, collagen, chitin, fibrin, hyaluronic acid, extracellular matrix mixture, silk, cellulose, dextran, agarose, agar, synthetic polypeptide, polylactic acid, polyleucine, alginic acid, polyglycolic acid, polymethyl methacrylate, polycyanoacrylate, polyacrylonitrile, polyurethan, polypropylene, polyethylene, polyvinyl chloride, ethylene-vinyl acetate copolymer, nylon and a combination thereof.

In another embodiment, the scaffold employed in the composite material according to the present invention is comprised of hydroxyapatite.

In one aspect, the present invention provides a kit for producing an agent capable of inducing the differentiation of osteoblasts comprising:

A) the composite material according to claim 64; and

B) at least one conventional osteoblast differentiation component selected from the group consisting of glucocorticoid, β-glycerophosphate and ascorbic acid.

In one aspect, the present invention provides a use of a chondrocyte capable of hypertrophication in the production of an agent capable of inducing differentiation of osteoblasts.

In one aspect, the present invention provides a use of a chondrocyte capable of hypertrophication and a conventional osteoblast differentiation component in production of an agent capable of inducing differentiation of osteoblasts.

In one aspect, the present invention provides a composition for enhancing or inducing osteogenesis in a biological organism, wherein the composition comprises a chondrocyte capable of hypertrophication, which has a potential for inducing differentiation of osteoblasts.

In one aspect, the present invention provides a composite material for enhancing or inducing osteogenesis in a biological organism, wherein the composite material comprises:

A) a chondrocyte capable of hypertrophication, which is capable of inducing differentiation of osteoblasts; and

B) a scaffold that is biocompatible with the biological organism.

In one aspect, the present invention provides a kit for enhancing or inducing osteogenesis in a biological organism comprising:

A) a chondrocyte capable of hypertrophication; and

B) at least one conventional osteoblast differentiation component selected from the group consisting of glucocorticoid, β-glycerophosphate and ascorbic acid.

In one aspect, the present invention provides a use of:

A) a chondrocyte capable of hypertrophication, which is capable of inducing differentiation of osteoblasts; and

B) a scaffold that is biocompatible with the biological organism, in the manufacture of an implant or a bone repairing material for enhancing or inducing osteogenesis in a biological organism.

In one aspect, the present invention provides a method for enhancing or inducing osteogenesis in a biological organism, comprising locating a composite material on a region in need thereof, wherein the composite material comprises a chondrocyte capable of hypertrophication, which has a potential for inducing differentiation of osteoblasts and a scaffold that is biocompatible with the biological organism.

### EFFECT OF THE INVENTION

The present invention provides a novel cell-regulating agent produced by a chondrocyte capable of hypertrophication and a producing method and method of use thereof. The novel cell-regulating agent can induce the differentiation of an undifferentiated cell into a more normal osteoblast. Such a cell-regulating agent produced by a chondrocyte capable of hypertrophication can lead undifferentiated cell to osteoblast, whereby making it possible to treat regions having a poor prognosis after implantation in prior art. Such a cellular function regulating agent produced by a chondrocyte capable of hypertrophication has not been provided by the prior art, but it is instead provided by the present invention for the first time. The present invention provides an agent capable of inducing the differentiation of osteoblasts for a board range of cells including conventional cell lines and/or cells distinct from conventional cells. Thus, the present invention could make a cell differentiate into an osteoblast, whereas the cell is incapable of being induced for differentiation using prior art agents.

These and other advantages of the present invention will be apparent from the drawings and a reading of the detailed description as follow.

### BRIEF DESCRIPTION OF THE DRAWINGS

FIG. 1A shows chondrocytes capable of hypertrophication diluted in cell suspension, inoculated to hydroxyapatite, and stained with alkaline phosphatase. The cells (1×10⁶ cells/ml) were inoculated to hydroxyapatite, incubated in 5% CO₂ incubator at 37° C. for one week, and stained with alkaline phosphatase. The hydroxyapatite was stained red with alkaline phosphatase.

FIG. 1B shows the result of toluidine blue staining of the samples stained with alkaline phosphatase in FIG. 1A. With toluidine blue, the same areas as FIG. 1A were stained blue, indicating the presence of cells.

FIG. 1C shows resting cartilage cells diluted in cell suspension, inoculated to hydroxyapatite, and stained with alkaline phosphatase. 1×10⁶ cells/ml were inoculated to hydroxyapatite, incubated in 5% CO₂ incubator at 37° C. for one week, and stained with alkaline phosphatase. The hydroxyapatite was not stained with alkaline phosphatase.

FIG. 1D shows the result of olivine blue staining the samples stained with alkaline phosphatase in FIG. 1C. With olivine blue, the hydroxyapatite stained blue, indicating the presence of cells.

FIG. 1E shows chondrocytes derived from articular cartilage diluted in cell suspension, inoculated to hydroxyapatite, and stained with alkaline phosphatase. 1×10⁶ cells/ml were inoculated to hydroxyapatite, incubated in a 5% CO₂ incubator at 37° C. for one week, and stained with alkaline phosphatase. The hydroxyapatite was not stained with alkaline phosphatase.

FIG. 1F shows the result of toluidine blue staining of samples stained with alkaline phosphatase in FIG. 1E. With toluidine blue, the hydroxyapatite was displayed in blue spotted staining, indicating the presence of cells.

FIG. 2 shows the alkaline phosphatase activity when the chondrocytes capable of hypertrophication derived from costa/costal cartilage were incubated in an MEM differentiation agent producing medium and an MEM growth medium, respectively, and the supernatants thereof were added to mouse C3H10T1/2 cells, which were then cultured. The value of alkaline phosphatase activity of the supernatant of the cell culture adding only an MEM differentiation agent producing medium was defined as 1. In the 4 week-old rat group, the relative value of the activity increased to about 4.1 times when a culture supernatant was collected 4 days after the medium was added, to about 5.1 times when a culture supernatant collected 1 week after the medium was added, to about 5.4 times when a culture supernatant collected 2 weeks after the medium was added, and to about 4.9 times when a culture supernatant collected 3 weeks after the medium was added. In the 8 week-old rat group, the relative value of the activity increased to about 2.9 times when a culture supernatant collected 4 days after the medium was added, to about 3.1 times when a culture supernatant collected 1 week after the medium was added, to about 3.8 times when a culture supernatant collected 2 weeks after the medium was added, and to about 4.2 times when a culture supernatant collected 3 weeks after the medium was added. There was little difference in the alkaline phosphatase activities of the groups of the 4 and 8 week-old rats between the supernatant of the cell culture adding an MEM growth medium and adding an MEM growth medium only. The following abbreviations show culture supernatants added. Four weeks old differentiation supernatant: culture supernatant from a chondrocyte capable of hypertrophication, derived from 4 weeks old rat, cultured in an MEM differentiation agent producing medium; Eight weeks old differentiation supernatant: culture supernatant from a chondrocyte capable of hypertrophication, derived from 8 weeks old rat, cultured in an MEM differentiation agent producing medium; Four weeks old growth supernatant: culture supernatant from a chondrocyte capable of hypertrophication, derived from 4 weeks old rat, cultured in an MEM growth medium; Eight weeks old growth supernatant: culture supernatant from a chondrocyte capable of hypertrophication, derived from 8 weeks old rat, cultured in an MEM growth medium;

FIG. 3A shows the result of alkaline phosphatase staining when the chondrocytes capable of hypertrophication derived from costa/costal cartilage were incubated in an MEM differentiation agent producing medium and an MEM growth medium, respectively, and the supernatants thereof were added to mouse C3H10T1/2 cells, which were then cultured. The mouse C3H10T1/2 cells were inoculated in 24-well plates (BME medium). Eighteen hours after inoculation, a fraction of each culture supernatant were added to the plates, then after 72 hours, stained with alkaline phosphatase. Upper column: it was confirmed in the case of adding culture supernatant from an MEM differentiation agent producing medium, the C3H10T1/2 cells were stained red and had the activities. Lower column: it was confirmed in the case of adding the culture supernatant from an MEM growth medium, the C3H10T1/2 cells were not stained and do not have the activities.

FIG. 3B shows the result of alkaline phosphatase staining when the chondrocytes capable of hypertrophication derived from costa/costal cartilage were incubated in an MEM differentiation agent producing medium, and the supernatants thereof were added to mouse C3H10T1/2 cells, which were then cultured. The mouse C3H10T1/2 cells were inoculated to hydroxyapatites (BME medium). Eighteen hours after inoculation a fraction of each culture supernatant was added to the hydroxyapatites and, after 72 hours, stained with alkaline phosphatase. It was confirmed in the case of adding the culture supernatant from an MEM differentiation agent producing medium, the samples were stained red and had the activities.

FIG. 3C shows the result of toluidine blue staining when the chondrocytes capable of hypertrophication derived from costa/costal cartilage were incubated in an MEM differentiation agent producing medium, and the supernatants thereof were added to mouse C3H10T1/2 cells, which were then cultured. The mouse C3H10T1/2 cells were inoculated to hydroxyapatites (BME medium). Eighteen hours after inoculation, the fraction of each culture supernatant was added to the hydroxyapatites and, after 72 hours, stained with toluidine blue. With toluidine blue, the samples were stained blue, indicating the presence of cells.

FIG. 3D shows the result of alkaline phosphatase staining when the chondrocytes capable of hypertrophication derived from costa/costal cartilage were incubated in MEM growth medium, and the supernatants thereof were added to mouse C3H10T1/2 cells, which were then cultured. The mouse C3H10T1/2 cells were inoculated to hydroxyapatites (BME medium). Eighteen hours after inoculation, a fraction of each culture supernatant was added to the hydroxyapatites and, after 72 hours, stained with alkaline phosphatase. It was confirmed that, in the case of adding culture supernatant from an MEM growth medium, the samples were not stained and do not have the activities.

FIG. 3E shows the result of toluidine blue staining when the chondrocytes capable of hypertrophication derived from costa/costal cartilage were incubated in MEM growth medium, and the supernatants thereof were added to mouse C3H10T1/2 cells, which were then cultured. The mouse C3H10T1/2 cells were inoculated to hydroxyapatites (BME medium). Eighteen hours after inoculation, a fraction of each culture supernatant was added the hydroxyapatites and, after 72 hours, stained with toluidine blue. With toluidine blue, the samples were stained blue, indicating the presence of cells.

FIG. 4 shows the alkaline phosphatase activity when the resting cartilage cells derived from costal cartilage were incubated in an MEM differentiation agent producing medium and an MEM growth medium, respectively, and each supernatant thereof was added to mouse C3H10T1/2 cells, which were then cultured. Adding the supernatant of the cell culture in an MEM differentiation agent producing medium and adding the supernatant of the cell culture in an MEM growth medium differed little from adding an MEM differentiation agent producing medium only and adding an MEM growth medium only, in the alkaline phosphatase activity. The following abbreviations show culture supernatants added. Eight weeks old differentiation supernatant: culture supernatant from a resting cartilage cell, derived from 8 weeks old rats, cultured in an MEM differentiation agent producing medium; Eight weeks old growth supernatant: culture supernatant from a resting cartilage cell, derived from 8 weeks old rats, cultured in an MEM growth medium. Each value was indicated by the value resulting from addition of only an MEM differentiation agent producing medium and the value resulting from the addition of only an MEM growth medium is defined as 1.

FIG. 5A shows the alkaline phosphatase activity when chondrocytes derived from articular cartilage were incubated in an MEM differentiation agent producing medium and an MEM growth medium, respectively, and each supernatant thereof was added to mouse C3H10T1/2 cells, which were then cultured. In the alkaline phosphatase activity of the chondrocytes derived from articular cartilage, adding the supernatant of the cell culture in an MEM differentiation agent producing medium and adding the supernatant of the cell culture in an MEM growth media differed little from adding MEM differentiation agent producing medium only and adding MEM growth medium only. The following abbreviations show culture supernatants added. Eight weeks old differentiation supernatant: culture supernatant from a articular cartilage cell, derived from 8 weeks old rats, cultured in an MEM differentiation agent producing medium; Eight weeks old growth supernatant: culture supernatant from a articular cartilage cell, derived from 8 weeks old rats, cultured in an MEM growth medium. Each value was indicated by the value resulting from addition of only MEM differentiation agent producing medium and the value resulting from the addition of only MEM growth medium is defined as 1.

FIG. 5B shows the alkaline phosphatase activity when the chondrocytes capable of hypertrophication derived from costa/costal cartilage were incubated in a HAM differentiation agent producing medium, and the supernatants thereof were added to mouse C3H10T1/2 cells, which were then cultured. The value was indicated by defining the value resulting from addition of only the HAM differentiation agent producing medium as 1. The alkaline phosphatase activity increased when the culture supernatant from costa/costal cartilage-derived chondrocytes capable of hypertrophication cultured in a HAM differentiation agent producing medium was added.

FIG. 5C shows the alkaline phosphatase activity when the chondrocytes capable of hypertrophication derived from costa/costal cartilage were incubated in HAM growth medium, and the supernatants thereof were added to mouse C3H10T1/2 cells, which were then cultured. The value was indicated by defining the value resulting from the addition of only HAM growth medium as 1. The alkaline phosphatase activity did not increase when the culture supernatant from costa/costal cartilage-derived chondrocytes capable of hypertrophication cultured in a HAM growth medium was added.

FIG. 6A shows the presence of the agent in the culture supernatant from chondrocytes capable of hypertrophication cultured in an MEM differentiation agent producing medium. The agent is capable of increasing the value of alkaline phosphatase activity in 3T3-Swiss albino cells and BALB/3T3 cells, and inducing the differentiation of undifferentiated cells into osteoblasts. On the other hand, FIG. 6A shows the absence of the agent in the culture supernatant from chondrocytes capable of hypertrophication cultured in an MEM growth medium. Furthermore, FIG. 6A shows the absence of the agent in the culture supernatant from chondrocytes incapable of hypertrophication cultured in an MEM differentiation agent producing medium or an MEM growth medium.

FIG. 6B shows the alkaline phosphatase activity when the chondrocytes capable of hypertrophication were incubated in the medium containing dexamethasone, β-glycerophosphate, ascorbic acid or a combination thereof as the conventional osteoblast differentiation components. The supernatants thereof were added to mouse C3H10T1/2 cells, which were then cultured. Dex: dexamethasone, BGP: β-glycerophosphate, Asc: ascorbic acid.

FIG. 7A shows the result of alkaline phosphatase staining when the chondrocytes capable of hypertrophication derived from costa/costal cartilage were incubated in an MEM differentiation agent producing medium, and a fraction of the supernatants thereof having a molecular weight over 50,000 was added to mouse C3H10T1/2 cells inoculated in 24-well plates, which were then cultured. The samples were stained red. It was confirmed that the agent capable of increasing the value of alkaline phosphatase activity is present in the fraction of the supernatants thereof having a molecular weight over 50,000.

FIG. 7B shows the result of alkaline phosphatase staining when the chondrocytes capable of hypertrophication derived from costa/costal cartilage were incubated in an MEM differentiation agent producing medium, and a fraction of the supernatants thereof having a molecular weight over 50,000 was added to mouse C3H10T1/2 cells inoculated with a hydroxyapatite, which were then cultured. The hydroxyapatite was stained red. It was confirmed that the agent capable of increasing the value of alkaline phosphatase activity is present in the fraction of the supernatants thereof having a molecular weight over 50,000.

FIG. 7C shows the result of alkaline phosphatase staining when the chondrocytes capable of hypertrophication derived from costa/costal cartilage were incubated in an MEM differentiation agent producing medium, and a fraction of the supernatants thereof having a molecular weight below 50,000 was added to mouse C3H10T1/2 cells inoculated in 24-well plates, which were then cultured. The agent capable of increasing the value of alkaline phosphatase activity was not observed in the fraction of the supernatants having a molecular weight below 50,000.

FIG. 7D shows the result of alkaline phosphatase staining when the chondrocytes capable of hypertrophication derived from costa/costal cartilage were incubated in an MEM differentiation agent producing medium, and a fraction of the supernatants thereof having a molecular weight below 50,000 was added to mouse C3H10T1/2 cells inoculated with a hydroxyapatite, which were then cultured. The agent capable of increasing the value of alkaline phosphatase activity was not observed in the fraction of the supernatants having a molecular weight below 50,000.

FIG. 8 shows the result of alkaline phosphatase staining when the chondrocytes capable of hypertrophication derived from mouse costa/costal cartilage and the resting cartilage cells derived from costal cartilage were incubated in an MEM differentiation agent producing medium and an MEM growth medium, respectively, and each supernatant thereof was added to mouse C3H10T1/2 cells, which were then cultured. The alkaline phosphatase activity increased 3.1 times, when the chondrocytes capable of hypertrophication were incubated in an MEM differentiation agent producing medium and the supernatants thereof were added. Adding the culture supernatant from chondrocytes capable of hypertrophication cultured in an MEM differentiation agent producing medium and adding the culture supernatants from costal cartilage-derived resting cartilage cells cultured in an MEM differentiation agent producing medium and an MEM growth medium, respectively, differed little from adding an MEM differentiation agent producing medium only and adding an MEM growth medium only, in the alkaline phosphatase activity. The following abbreviations show culture supernatants added. GC differentiation supernatant: Culture supernatant from chondrocytes capable of hypertrophication cultured in an MEM differentiation agent producing medium, GC growth supernatant: Culture supernatant from chondrocytes capable of hypertrophication cultured in an MEM growth medium, RC differentiation supernatant: Culture supernatant from resting cartilage cells cultured in an MEM differentiation agent producing medium, RC growth supernatant: Culture supernatant from resting cartilage cells cultured in an MEM growth medium. Each values were indicated by the value resulting from the addition of only MEM differentiation agent producing medium and the value resulting from the addition of only MEM growth medium is defined as 1.

FIG. 9 shows the effect of a culture medium on the differentiation of the undifferentiated cells into osteoblasts. The chondrocyte capable of hypertrophication, resting cartilage cells and articular cartilage cells were cultured in an MEM differentiation agent producing medium and an MEM growth medium, respectively. Each culture supernatant thereof was added to mouse C3H10T1/2 cells, which were then cultured, and thereby the alkaline phosphatase activities were measured. A HAM medium and an MEM medium were used as the medium for culturing mouse C3H10T1/2 cells. When a HAM medium was used in the culture of mouse C3H10T1/2 cells, alkaline phosphatase activity was only observed in culture supernatant from a chondrocyte capable of hypertrophication cultured in an MEM differentiation agent producing medium. When an MEM medium was used in the culture of mouse C3H10T1/2 cells, the same result was observed. The following abbreviations show culture supernatants added. GC differentiation supernatant: Culture supernatant from chondrocytes capable of hypertrophication cultured in an MEM differentiation agent producing medium, GC growth supernatant: Culture supernatant from chondrocytes capable of hypertrophication cultured in an MEM growth medium, RC differentiation supernatant: Culture supernatant from resting cartilage cells cultured in an MEM differentiation agent producing medium, RC growth supernatant: Culture supernatant from resting cartilage cells cultured in an MEM growth medium, AC differentiation supernatant: Culture supernatant from articular cartilage cells cultured in an MEM differentiation agent producing medium, AC growth supernatant: Culture supernatant from articular cartilage cells cultured in an MEM growth medium. Each values were indicated by the value resulting from the addition of only MEM differentiation agent producing medium and the value resulting from the addition of only MEM growth medium is defined as 1.

FIG. 10 shows the alkaline phosphatase activity when the agent, which was produced by a chondrocyte capable of hypertrophication, inducing the differentiation of undifferentiated cells into osteoblasts was heated. The culture supernatant from a chondrocyte capable of hypertrophication cultured in an MEM differentiation agent producing medium was heated for 3 minutes in boiling water. Non-heated culture supernatant, heated culture supernatant, and an MEM differentiation agent producing medium alone was individually added to mouse C3H10T1/2 cells and the alkaline phosphatase activities were measured after 72 hours. The alkaline phosphatase activity did not increase when the culture supernatant was heated. It was confirmed that the agent inducing the differentiation of undifferentiated cells into osteoblasts was degenerated (inactivated) by heat treatment. The following abbreviations show culture supernatants added. GC heated: Treated culture supernatant from chondrocytes capable of hypertrophication cultured in an MEM differentiation agent producing medium, GC differentiation supernatant: Culture supernatant from chondrocytes capable of hypertrophication cultured in an MEM differentiation agent producing medium, differentiation supernatant only: an MEM differentiation agent producing medium only. Each values were indicated by defining the value resulting from the addition of only MEM differentiation agent producing medium as 1.

FIG. 11A shows the activity of TGFβ in an MEM differentiation agent producing medium including the agent of the present invention.

FIG. 11B shows the activity of BMP in an MEM differentiation agent producing medium including the agent of the present invention.

### BEST MODE FOR CARRYING OUT THE INVENTION

The present invention is described hereafter. It is to be understood that, unless otherwise described, singular representations throughout the present specification include the concept of plural thereof. Therefore, it is to be understood that, unless particularly described, singular articles such as “a”, “an” and “the” in the English language, “un”, “une”, “le” and “la” in the French language, and “ein”, “eine”, “der”, “die” and “das”, and the like, in the German language, or others include the concept of plural. It should be also understood that terms used herein have the definitions ordinarily used in the art unless otherwise mentioned. Therefore, unless otherwise defined, all technical and scientific terms used herein have the same meaning as that commonly understood by those skilled in the art. Otherwise, the present application (including definitions) takes precedence.

(Definition of Terms)

The definitions of the terms particularly used herein are listed below.

(Cell)

A “growth cartilage cell” or a “growth chondrocyte” is interchangeably used herein to refer to a cell in a tissue which forms bone during developmental or growth stages, and periods of bone recovery or proliferation (i.e., growth cartilage). A growth cartilage cell generally refers to a tissue which forms bone during growth stage, while it herein means a tissue which forms bone during developmental or growth stages, and periods of bone proliferation or recovery. The growth cartilage cell is also referred to as cartilage capable of hypertrophication, calcified cartilage or epiphysial (line) cartilage. When using a growth cartilage cell in humans, cells derived from a human are preferred, but it is also possible to use non-human cells since problems such as immunological rejection can be avoided using techniques well-known in the art.

The growth cartilage cell according to the present invention is derived from a mammal, preferably from a human, a mouse, a rat or a rabbit.

The growth cartilage cell according to the present invention can be sampled from the chondro-osseous junction of costa, epiphysial line of long bone (e.g., femoris, tibia, fibula, humerus, ulna, and radius), epiphysial line of vertebra, zone of proliferating cartilage of hand bone, foot bone, breast bone and others, perichondrium, bone primordium formed from cartilage of fetus, the callus region of a healing bone-fracture, and the cartilaginous part of bone proliferation phase. These growth cartilage cells can be prepared, for example, by the methods described in the Examples of the present specification.

“A chondrocyte capable of hypertrophication” according to the present specification refers to a cell which can undergo hypertrophic growth in the future. A chondrocyte capable of hypertrophication includes a “growth cartilage cell” collected from a living organism, as well as any other cells capable of hypertrophication determined by a method for determining “the ability of hypertrophication” defined hereinafter.

The chondrocyte capable of hypertrophication according to the present invention is derived from a mammal, preferably a human, a mouse, a rat, or a rabbit. When using chondrocytes capable of hypertrophication in humans, the cells are preferably derived from a human, but it is also possible to use non-human cells since problems such as immunological rejection can be avoided using techniques well-known in the art. The chondrocyte capable of hypertrophication according to the present invention can be obtained, for example, from the chondro-osseous junction of costa, epiphysial line of long bone (e.g., femoris, tibia, fibula, humerus, ulna, and radius), epiphysial line of vertebra, zone of proliferating cartilage of e.g., hand bones, foot bones, sterna, perichondrium, bone primordium formed from cartilage of fetus, the callus region of a healing bone-fracture, and the cartilaginous part of bone proliferation phase. The chondrocyte capable of hypertrophication according to the present invention can be obtained by inducing the differentiation of an undifferentiated cell.

The chondrocyte capable of hypertrophication according to the present invention may be a chondrocyte obtained from any region other than above-described. Because a bone formed by endochondral ossification (enchondral ossification) is formed by the same mechanism irrespective of the region of the body. In other words, the chondrocyte is formed and substituted with a bone. The major part of bone other than cranium and clavicle is formed by the endochondral ossification (enchondral ossification). Therefore, there are chondrocytes capable of hypertrophication in the major part of bone other than cranium and clavicle on the body. The chondrocyte capable of hypertrophication is capable of osteogenesis.

The chondrocyte capable of hypertrophication may be morphologically characterized by hypertrophy.

“Hypertrophy” according to the present specification can be determined morphologically under a microscope. The hypertrophy of a cell refers to a cell observed adjacent to the growth layer, which aligns in a columnar state, or alternatively refers to a cell that is larger than the surrounding cells.

Cells are determined to be capable of hypertrophication when a significant increase in size thereof is observed by preparing a pellet of the cells centrifuged in HAM's F12 culture medium which contains 5×10⁵ cells, culturing the pellet for a pre-determined period, and comparing the sizes of the cells before and after cell culture under a microscope.

“Resting cartilage cells” and “resting chondrocytes” are interchangeably, according to the present specification, referred to a cartilage cell or a chondrocyte located in the region apart from the chondro-osseous junction of the costa (zone of proliferating cartilage), which is a tissue that exists as cartilage throughout the entire lifetime. A cell located in the resting cartilage is referred to as a resting cartilage cell. An “articular cartilage cell” according to the present specification refers to a cell in the cartilaginous tissue (articular cartilage) located on an articular surface.

The chondrocyte according to the present specification is determined by identifying the expression of at least one marker selected from the group consisting of type II collagen, cartilage proteoglycan (aglycan) or components thereof, hyaluronic acid, type IX collagen, type XI collagen, and chondromodulin. Among chondrocytes, a cell capable of hypertrophication is further determined by identifying the expression of at least one marker selected from the group consisting of type X collagen, alkaline phosphatase, and osteonectin. Chondrocytes not expressing any of type X collagen, alkaline phosphatase or osteonectin, are determined as not having the ability of hypertrophication potency. Therefore, the chondrocyte capable of hypertrophication described herein may be also determined by identifying the expression of at least one selected from chondrocyte markers and of at least one selected from markers for hypertrophic chondrocytes, instead of observing morphological hypertrophy. The localization or expression of these markers is identified by any method of analyzing proteins or RNA extracted from cultured cells, such as specific staining, immunohistochemical methods, in situ hybridization, Western blotting, or PCR.

A “chondrocyte marker” according to the present specification refers to any substance whose localization or expression in a chondrocyte aids in the identification of the chondrocyte. Preferably, it refers to any substance which can be used to identify the chondrocyte by their localization or expression (for example, localization or expression of type II collagen, cartilage proteoglycan (aglycan) or components thereof, hyaluronic acid, type IX collagen, type XI collagen, or chondromodulin). A “marker for chondrocyte capable of hypertrophication” as used herein refers to any substance whose localization or expression in a chondrocyte capable of hypertrophication aids in the identification of the chondrocyte. Preferably, it refers to any substance which can be used to identify the chondrocyte capable of hypertrophication by their localization or expression (for example, localization or expression of type X collagen, alkaline phosphatase and osteonectin).

“Cartilage proteoglycan” according to the present specification refers to a macromolecule, wherein the plurality of glucosaminoglycans, such as chondroitin tetrasulfate, chondroitin hexasulfate, keratan sulfate, O-linked oligosaccharide, N-linked oligosaccharide and others, are combined with a core protein. The cartilage proteoglycan further binds to hyaluronic acid via a linkage protein to form cartilage proteoglycan aggregates. In the cartilaginous tissue, the glucosaminoglycan is rich and occupies 20-40% of dry weight of the tissue. Cartilage proteoglycan is also referred to as aglycan.

“Bone proteoglycan” according to the present specification refers to a macromolecule which has a smaller molecular weight than cartilage proteoglycan, wherein glucosaminoglycans such as chondroitin sulfate, dermatan sulfate, O-linked oligosaccharides, N-linked oligosaccharides and others, are combined with a core protein. In the bone tissue, glucosaminoglycan occupies 1% or less dry weight of decalcified bone. Bone proteoglycan may include decorin and biglycan.

An “osteoblast” according to the present specification is a cell which locates on the bone matrix and forms and calcifies the bone matrix. Osteoblasts are cells of 20-30 μm diameter and in cubic or columnar forms. As used herein, an osteoblast may include a “preosteoblast”, which is a precursor cell of an osteoblast.

Osteoblasts are determined by the expression of at least one marker selected from the group consisting of type I collagen, bone proteoglycan (e.g., decorin, biglycan), alkaline phosphatase, osteocalcin, matrix Gla protein, osteoglycin, osteopontin, bone sialic acid protein, osteonectin and pleiotrophin. Additionally, osteoblasts can be determined by identifying chondrocyte markers (such as type II collagen, cartilage proteoglycan (aglycan) or components thereof, hyaluronic acid, type IX collagen, type XI collagen, or chondromodulin), that are not expressed therein. These markers are identified by their localization or expression by any methods of analyzing proteins or RNA extracted from cultured cells, such as specific staining, immunohistochemical methods, in situ hybridization, Western blotting, or PCR.

“Osteoblast marker” according to the present specification refers to any substance whose localization or expression in an osteoblast aids in the identification of the osteoblast. Preferably, it refers to any substance which can be used to identify osteoblasts by their localization or expression (for example, localization or expression of type I collagen, Bone proteoglycan (e.g., decorin, biglycan), alkaline phosphatase, osteocalcin, matrix Gla protein, osteoglycin, osteopontin, bone sialic acid protein, osteonectin or pleiotrophin). Osteoglycin is referred to as osteoinductive factor (OIF). Osteopontin is referred to as BSP-1 or 2ar. Bone sialic acid protein is referred to as BSP-II. Pleiotrophin is referred to as osteoblast specific protein (OSF-1). Osteonectin is referred to as SPARC, or BM-40.

Osteoblasts may be identified, for example, by:

determining a cell to be positive for a marker that only identifies osteoblasts;

determining a cell to be positive for a marker identifying osteoblasts and chondrocytes capable of hypertrophication, while not identifying chondrocytes, and determining said cell to be positive for a marker that identifies osteoblasts and chondrocytes, while not identifying chondrocytes capable of hypertrophication;

determining a cell to be positive for a marker identifying osteoblasts and chondrocytes capable of hypertrophication, but to be negative for a marker that does not identify osteoblasts, while identifying chondrocytes capable of hypertrophication; or

determining a cell to be positive for a marker identifying osteoblasts and chondrocytes as positive, but to be negative for a marker that does not identify osteoblasts, while identifying chondrocytes.

Chondrocytes capable of hypertrophication may be identified, for example, by:

determining a cell to be positive for a marker that only identifies chondrocytes capable of hypertrophication;

determining a cell to be positive for a marker identifying chondrocytes capable of hypertrophication and osteoblasts, while not identifying chondrocytes, and determining said cell to be positive for a marker that identifies chondrocytes capable of hypertrophication and chondrocytes, while not identifying osteoblasts;

determining a cell to be positive for a marker identifying chondrocytes capable of hypertrophication and osteoblasts, but to be negative for a marker that does not identify chondrocytes capable of hypertrophication, while identifying osteoblasts; or

determining a cell to be positive for a marker identifying chondrocytes capable of hypertrophication and chondrocytes, but to be negative for a marker that does not identify chondrocytes capable of hypertrophication, while identifying chondrocytes.

Chondrocytes (without the ability of hypertrophication) may be identified, for example, by:

determining a cell to be positive for a marker that only identifies chondrocytes;

determining a cell to be positive for a marker identifying chondrocytes and osteoblasts, while not identifying chondrocytes capable of hypertrophication, and determining said cell to be positive for a marker that identifies chondrocytes and chondrocytes capable of hypertrophication, while not identifying osteoblasts;

determining a cell to be positive for a marker identifying chondrocytes and osteoblasts, but to be negative for a marker that does not identify chondrocytes, while identifying osteoblasts; or

determining a cell to be positive for a marker identifying chondrocytes and chondrocytes capable of hypertrophication, but to be negative for a marker that does not identify chondrocytes, while identifying chondrocytes capable of hypertrophication.

Chondrocytes, chondrocytes capable of hypertrophication and osteoblasts may be identified herein, for example, using the combinations of markers listed below, in Table A:

| | | chondrocyte | |
|---|---|---|---|
| | chondro- | capable of | osteo- |
| | cyte | hypertrophication | blast |
| type II collagen, cartilage | + | + | − |
| proteoglycan (aglycan), | | | |
| hyaluronic acid, type IX | | | |
| collagen, type XI collagen, | | | |
| chondromodulin | | | |
| type X collagen | − | + | − |
| alkaline phosphatase, | − | + | + |
| osteonectin | | | |
| type I collagen, bone | − | − | + |
| proteoglycan (e.g., decorin, | | | |
| biglycan), osteocalcin, | | | |
| matrix Gla protein, | | | |
| osteoglycin, osteopontin, | | | |
| bone sialic acid protein, | | | |
| pleiotrophin | | | |

+: expressed
−: not expressed

As used herein, the term “induce to differentiation” refers to the development process of parts in a biological organism such as cells, tissues and organs, wherein the development process is a process of inducing to the formation of tissues or organs having specific features. The terms “differentiation” and “induce to differentiation” are mainly used in embryology, development biology and the like. The tissues and organs in a biological organism are formed by the divisions of a fertilized ovum consisting of a single cell until one reaches adulthood. It is difficult to distinguish between cells and cell populations in the early development of a biological organism which is before differentiation or is not well differentiated, because the cells and cell populations do not have any morphological or functional feature at all. Such condition is referred to “undifferentiation”. Furthermore, “differentiation” occurs in an organ, and thereby various cells comprising of the organ develop to a specific cell and a cell population. This is referred to as differentiation within the organ in organogenesis. Such an induction to development is referred to as inducing to differentiation.

As used herein, “ability to inducing the differentiation into osteoblast” refers to the ability to differentiate an undifferentiated cell, preferably an embryonic stem (ES) cell, an embryonic germ (EG) stem cell or a tissue stem cell, more preferably a mesenchymal stem cell to an osteoblast. The ability of inducing differentiation of an osteoblast may be decided by measuring an osteoblast marker (e.g., alkaline phosphatase). Specifically, it is determined, that the agent of the present invention is capable of inducing differentiation to an osteoblast, by increasing the value of alkaline phosphatase activity of a C3H10T1/2 cell (e.g., alkaline phosphatase activity of whole the cell) which is exposed to the agent in Eagle's basal medium to be higher by more than about one times that of the cell cultured in Eagle's basal medium without the agent, wherein the alkaline phosphatase activity is determined by the following steps: A) determining the two absorbances at 405 nm, wherein, for one absorbance of the 100 μl samples with or without the agent, 50 μl of 4 mg/ml p-nitrophenyl phosphate and 50 μl of alkali buffer (Sigma, A9226) are added, respectively, and reacted at 37° C. for 15 minutes, and 50 μl 1N NaOH is added to terminate the reaction, While for the other absorbance of the samples, a further 20 μl concentrated hydrochloric acid is added; and B) calculating the difference in absorbance before and after the addition of the concentrated hydrochloric acid, wherein the difference in absorbance is an indicator of the alkaline phosphatase activity. Furthermore, it is determined, that the agent of the present invention is capable of inducing the differentiation to an osteoblast, by increasing the value of the alkaline phosphatase (ALP) activity (e.g., alkaline phosphatase activity of whole the cell) of a C3H10T1/2 cell when the C3H10T1/2 cell is exposed to the agent in Eagle's basal medium. The alkaline phosphatase activity is determined by the following steps: A) determining two absorbances at 405 nm, wherein, for one absorbance of the two 100 μl samples with or without the agent, 50 μl of 4 mg/ml p-nitrophenyl phosphate and 50 μl of alkali buffer (Sigma, A9226) are added, respectively, and reacted at 37° C. for 15 minutes, and 50 μl 1N NaOH is added to terminate the reaction, while for the other absorbance of the samples, a further 20 μl concentrated hydrochloric acid is added; and B) calculating the difference in absorbance before and after the addition of the concentrated hydrochloric acid, wherein the difference in absorbance is an indicator of the alkaline phosphatase activity. 0-10 m M of p-nitro phenol solutions are prepared in each experiment to measure the absorbance and to thereby plot a linear calibration curve of their values using concentration as the X axis and absorbance as the Y axis. The absolute value is then calculated from the absorbance using this calibration curve.

As described herein, “ability of inducing the differentiation to an osteoblast” for an undifferentiated cell (e.g., embryonic stem cells, embryonic germ stem cells, mesenchymal stem cells, hematopoietic stem cells, vascular stem cells, hepatic stem cells, pancreatic (common) stem cells, neural stem cells) refers to the ability to differentiate an undifferentiated cell to an osteoblast. For example, the ability to differentiate to an osteoblast may include the ability to differentiate an undifferentiated cell which is not differentiated by glucocorticoid, β-glycerophosphate and ascorbic acid into an osteoblast. The ability of differentiate to an osteoblast may be decided by the following experiment. A density of 1.25×10⁴ cells/cm² of subject cells are inoculated evenly in 24-well plates (Becton Dickinson, 2.5×10⁴/well), and cultured in a 5% CO₂ incubator at 37° C. for 72 hours, and measured for the induced or increasing expression of at least one osteoblast marker.

As described herein, an “undifferentiated cell” refers to a cell which has not reached terminal differentiation or a cell which has the ability to differentiate. As used herein, the undifferentiated cell may be a stem cell (e.g., an embryonic stem cell, an embryonic germ stem cell or a tissue stem cell), which may be a mesenchymal stem cell, a hematopoietic stem cell, a vascular stem cell, a hepatic stem cell, a pancreatic (common) stem cell, a neural stem cell. The undifferentiated cell further includes all cells on the way of differentiation, which may be a C3H10T1/2 cell, an ATDC5 cell, a 3T3-Swiss albino cell, a BALB/3T3 cell, a NIH3T3 cell and the like. An undifferentiated cell used in the present invention may be any cell in which the cell can differentiate into osteoblast.

As used herein, the term “stem cell” refers to a cell capable of self-replication and pluripotency (i.e., multipotency). Typically, stem cells can regenerate an injured tissue. Stem cells used herein may be, but not limited to, embryonic stem (ES) cells, embryonic germ (EG) stem cells or tissue stem cells (also called tissular stem cells, tissue-specific stem cells, or somatic stem cells). A stem cell may be an artificially produced cell (e.g., a fusion cell, a reprogrammed cell, or the like used herein) as long as it can have the above-described abilities. Embryonic stem cells are pluripotent stem cells derived from early embryos. An embryonic stem cell was first established in 1981, which has been applied to the production of knockout mice since 1989. In 1998, a human embryonic stem cell was established and currently becoming available for regenerative medicine. It is believed that an embryonic germ cell is formed by dedifferentiation by exposing a primordial germ cell to a specific surrounding agent. While an embryonic germ cell has a property as an embryonic stem cell, the embryonic germ cell holds a part of a property of the primordial germ cell derived from. Tissue stem cells are present in tissue, have a lower level of pluripotency than embryonic stem cells, and have a relatively limited level of differentiation, unlike embryonic stem cells. Generally, stem cells have an undifferentiated intracellular structure, a high nucleus/cytoplasm ratio and few intracellular organelles. As used herein, stem cells may be preferably mesenchymal stem cells, though tissue stem cells, embryonic germ cells or embryonic stem cells may also be employed depending on the circumstances.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

The origin of a stem cell is categorized into the ectoderm, endoderm, or mesoderm. Stem cells of ectodermal origin are mostly present in the brain, including neural stem cells. Stem cells of endodermal origin are mostly present in bone marrow, including blood vessel stem cells, hematopoietic stem cells, mesenchymal stem cells, and the like. Stem cells of mesoderm origin are mostly present in organs, including liver stem cells, pancreas stem cells, and the like.

“Mesenchymal stem cell” as described herein refers to a stem cell observed in mesenchymal tissue. The mesenchymal tissue includes, but is not limited to bone marrow, adipose tissue, vascular endothelium, smooth muscle, cardiac muscle, skeletal muscle, cartilage, bone, and ligament. Mesenchymal stem cells are typically derived from bone marrow, adipose tissue, synovial tissue, muscular tissue, peripheral blood, placental tissue, menstrual blood, or cord blood.

A “growth medium” according to the present specification, refers to a medium containing a basal medium, antibiotics (e.g., penicillin and streptomycin), an antibacterial agent (e.g., amphotericin B) and a serum component (e.g., human serum, bovine serum, fetal bovine serum). Typically, the serum component may make up to 20% of the medium. Furthermore, if the base medium is a minimum essential medium (MEM), the medium is referred to as an “MEM growth medium”. If the base medium is a HAM medium, the medium is referred to as a “HAM growth medium”.

As described herein, a “differentiation agent producing medium” refers to a medium which comprises a basic medium, and at least one conventional osteoblast differentiation component selected from the group consisting of glucocorticoid, β-glycerophosphate and ascorbic acid. The differentiation agent producing medium may comprise at least one conventional osteoblast differentiation component selected from the group consisting of β-glycerophosphate and ascorbic acid. The differentiation agent producing medium may comprise all of glucocorticoid, β-glycerophosphate and ascorbic acid as conventional osteoblast differentiation component. Preferably, the differentiation agent producing medium comprises Minimum Essential Medium (MEM) as the basic component, and β-glycerophosphate and ascorbic acid as the conventional osteoblast differentiation components. Preferably, “the differentiation agent producing medium” may further comprise a serum component (e.g., human serum, bovine serum, fetal bovine serum). Typically, the serum component may make up to 20% of the medium. Furthermore, if the base medium is a minimum essential medium (MEM), the medium is referred to as an “MEM differentiation agent producing medium”. If the base medium is a HAM medium, the medium is referred to as a “HAM differentiation agent producing medium”. It has not been shown that the differentiation agent producing medium itself have the ability to differentiate a C3H10T1/2 cell, a 3T3-Swiss albino cell, a Balb 3T3 cell into an osteoblast. Therefore, it is believed that an agent in the present invention is different from the components included in the differentiation agent producing medium.

As described herein, a “conventional osteoblast differentiation component” has been proposed by Maniatopoulos, C et. al.: Bone formation in vitro by stromal cells obtained from bone marrow of young adult rats. Cell Tissue Res., 254: 317-330, 1988. Therefore, the conventional osteoblast differentiation component is a component used to differentiate a bone marrow cell into an osteoblast, and refers to a combination of glucocorticoid, β-glycerophosphate and ascorbic acid.

As descried herein, “glucocorticoid” is an adrenal cortex hormone, and is a generic name of a steroid hormone associated with the saccharometabolism. Glucocorticoid is known as a component to differentiate a bone marrow cell to an osteoblast (Maniatopoulos, C. et. al.: Bone formation in vitro by stromal cells obtained from bone marrow of young adult rats. Cell Tissue Res., 25 4: 317-330, 1988.), but not the same effect of differentiation as described above. Glucocorticoid is also referred to as glycocorticoid. Typically, glucocorticoid includes, but not limited to, dexamethasone, betamethasone, predonisolone, predonisone, cortisone, cortisol, corticosterone. Preferably, dexamethasone is used. Chemically synthesized substance having the same effect as native glucocorticoid may be included. These typical glucocorticoids are used in the culture of a chondrocyte capable of hypertrophication together with β-glycerophosphate and ascorbic acid, and thereby produce an agent having the activity of differentiating a C3H10T1/2 cell to an osteoblast. Therefore, in the p resent invention, these typical glucocorticoids can be incorporated in the differentiation agent producing medium. The glucocorticoid may comprise a concentrations of 0.1 nM-10 mM, preferably 10-100 nM in the differentiation agent producing medium.

As described herein, “β-glycerophosphate” is a generic name of a salt attached to a phosphate group at the β-position of glycerophosphoric acid (C₃H₅(OH)₂OPO₃H₂). The salt may include calcium salt or sodium salt. β-glycerophosphate is known as a component to differentiate a bone marrow cell into an osteoblast (Maniatopoulos, C. et. al.: Bone formation in vitro by stromal cells obtained from bone marrow of young adult rats. Cell Tissue Res., 254: 317-330, 1988.), but not the same effect of differentiation as described above. The β-glycerophosphate is used in the culture of a chondrocyte capable of hypertrophication together with glucocorticoid and ascorbic acid, thereby produces an agent having the activity of differentiating a C3H10T1/2 cell to an osteoblast. Therefore, in the present invention, β-glycerophosphate can be incorporated in the differentiation agent producing medium. β-glycerophosphate may comprise a concentration of 0.1 m M-1 M, preferably 10 mM, in the differentiation agent producing medium.

As described herein, “ascorbic acid” is a white water-soluble vitamin crystalline. The ascorbic acid is comprised in the majority of plants, especially citrus fruit. The ascorbic acid is also referred to as vitamin C. Ascorbic acid is known as a component to differentiate a bone marrow cell into an osteoblast (Maniatopoulos, C. et. al.: Bone formation in vitro by stromal cells obtained from bone marrow of young adult rats. Cell Tissue Res., 254: 317-330, 1988), but not the same effect of differentiation as described above. Ascorbic acid, according to the present invention, may include ascorbic acid and derivatives thereof. Ascorbic acid includes, but not limited to, L-ascorbic acid, L-ascorbic acid sodium, L-ascorbyl palmitate, L-ascorbyl stearate, L-ascorbic acid 2-glucoside, ascorbyl acid phosphate magnesium, and ascorbic acid glucoside. Chemically synthesized substances having the same effect as native ascorbic acid may be included. These typical ascorbyl acids are used in the culture of a chondrocyte capable of hypertrophication together with glucocorticoid and β-glycerophosphate, thereby producing an agent having the activity of differentiating a C3H10T1/2 cell into an osteoblast. Therefore, in the present invention, these typical ascorbic acids can be incorporated in the differentiation agent producing medium. The ascorbic acid may be comprised a concentration of 0.1 μg/ml-5 mg/ml, preferably 10-50 μg/ml, in the differentiation agent producing medium.

### DESCRIPTION OF THE PREFERRED EMBODIMENTS

The best modes of the present invention are described below. It is appreciated that the embodiments provided below are be provided for the purpose of a better understanding of the invention. The scope of the invention should not be limited to the following descriptions. Therefore, it is apparent that those skilled in the art can read the descriptions herein and modify them appropriately within the scope of the present invention.

(An Agent Capable of Inducing Differentiation of an Osteoblast)

In one aspect, the present invention provides an agent which can be obtained by culturing a chondrocyte capable of hypertrophication in a differentiation agent producing medium. It has been known that osteogenesis is a preferred method to treat diseases associated with decreasing osteogenesis, damage of the bone or bone deficits, and that the osteoblast plays an important role in osteogenesis. However, the osteoblast used in these treatments has not been provided with safety, low-cost and stability. As such, there is an anxiety over the property and function thereof. The present invention has an effect which can differentiate an undifferentiated cell to an osteoblast by exposing the undifferentiated cell, preferably a mesenchymal stem cell, to an agent produced by a chondrocyte capable of hypertrophication. The osteoblast can be provided with safety, low-cost and stability using the agent. Such an agent can be obtained by culturing a chondrocyte capable of hypertrophication in a differentiation agent producing medium. The differentiation agent producing medium comprises Minimum Essential Medium (MEM) or HAM medium as a basic medium, and at least one conventional osteoblast differentiation component selected from the group consisting of β-glycerophosphate and ascorbic acid. The differentiation agent producing medium may further comprise glucocorticoid. It has not been shown that the differentiation agent producing medium itself dose have the ability to differentiate a C3H10T1/2 cell, a 3T3-Swiss albino cell, or a Balb/3T3 cell into an osteoblast.

In one preferred embodiment, the agent of the present invention is capable of increasing the value of the alkaline phosphatase (ALP) activity (e.g., alkaline phosphatase activity of a whole cell) of a C3H10T1/2 cell which is exposed to the agent in Eagle's basal medium to be higher than those of the cell cultured in Eagle's basal medium without the agent by more than about one times. The alkaline phosphatase activity is determined by the following steps: A) determining two absorbances at 405 nm, wherein for one absorbance of the 100 μl samples with or without the agent, 50 μl of 4 mg/ml p-nitrophenyl phosphate and 50 μl of alkali buffer (Sigma, A9226) are added, respectively, and reacted at 37° C. for 15 minutes, and 50 μl 1N NaOH was added to terminate the reaction, while for the other absorbance of the samples, a further 20 μl concentrated hydrochloric acid was added; and B) calculating the difference of absorbance before and after the addition of the concentrated hydrochloric acid, the difference in absorbance is an indicator of the alkaline phosphatase activity. Preferably, the alkaline phosphatase activity shows an increase by at least 2 times, at least 3 times, at least 4 times, at least 5 times, at least 6 times, at least 7 times, at least 8 times, at least 9 times, at least 10 times, at least 11 times, at least 12 times, or at least 13 times.

In one preferred embodiment, the agent of the present invention is capable of increasing the value of the alkaline phosphatase (ALP) activity (e.g., alkaline phosphatase activity of a whole cell) of a C3H10T1/2 cell when the C3H10T1/2 cell is exposed to the agent in Eagle's basal medium. The alkaline phosphatase activity is determined by the following steps: A) determining two absorbances at 405 nm, wherein, for one absorbance of the 100 μl samples with or without the agent, 50 μl of 4 mg/ml p-nitrophenyl phosphate and 50 μl of alkali buffer (Sigma, A9226) are added, respectively, and reacted at 37° C. for 15 minutes, and 50 μl 1N NaOH was added to terminate the reaction, while for the other absorbance of the samples, a further 20 μl concentrated hydrochloric acid was added; and B) calculating the difference in absorbance before and after the addition of the concentrated hydrochloric acid, the difference in absorbance is an indicator of the alkaline phosphatase activity. Preferably, the alkaline phosphatase activity shows an increase by at least 2 times, at least 3 times, at least 4 times, at least 5 times, at least 6 times, at least 7 times, at least 8 times, at least 9 times, at least 10 times, at least 11 times, at least 12 times, or at least 13 times.

As described herein “agent” or “factor” may be any substance or component as long as it achieves the purpose intended. For example, an agent capable of inducing the differentiation of osteoblast of the present invention may be, for example, a protein, a polypeptide, an oligopeptide, a peptide, an amino acid, a nucleic acid, a polysaccharide, a lipid, an organic low molecular weight molecule or a composite molecule thereof. In the present specification, an agent capable of inducing the differentiation of osteoblast, obtainable by culturing a chondrocyte capable of hypertrophication in the differentiation agent producing medium, may be simplex or complex as long as it can have the activity itself. It is understood that an agent obtained by another method or an agent of a distinct formation is interchangeably used in the present invention, if the agent has the same activity of the agent capable of inducing the differentiation of undifferentiated cells into osteoblast of the present invention. Such an agent can be identified using a common technique by those skilled in the art, based on the disclosure of the present specification, in addition to agents basically identified in the Examples.

The present agent has the ability to increase the expression of an osteoblast specific substance selected from the group consisting of type I collagen, bone proteoglycan (e.g., decorin, biglycan), alkaline phosphatase, osteocalcin, matrix Gla protein, osteoglycin, osteopontin, bone sialic acid protein, osteonectin and pleiotrophin. Therefore, the agent having the ability to differentiate an osteoblast herein is characterized by increasing the alkaline phosphatase activity of an undifferentiated cell in the enzyme activity, or the agent having the ability to express at least one osteoblast marker in the undifferentiated cell at the level of the gene expression or protein expression. In a preferred embodiment, the agent capable of inducing the differentiation of osteoblast in the present invention can be identified by measuring the increase in alkaline phosphatase activity, and the expression or localization of an osteoblast marker in an undifferentiated cell. In another embodiment of the present invention, an agent capable of inducing the differentiation of osteoblast is prevented to induce the differentiation of undifferentiated cells into osteoblasts by heating for 3 minutes in boiling water (generally, including about 96° C.-100° C., e.g., about 96° C., about 97° C., about 98° C., about 99° C. and about 100° C.). The boiling is confirmed by observation. Preventing the induction of the differentiation of undifferentiated cells into osteoblasts is referred as a state that does not substantially increase the localization or expression of a osteoblast marker. In another embodiment in the present invention, an agent capable of inducing the differentiation of osteoblast is prevented to induce the alkaline phosphatase activity by heating for 3 minutes in boiling water. Preventing the induction of an alkaline phosphatase activity of undifferentiated cells is referred as a state that does not substantially increase the alkaline phosphatase activity.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. There terms, as used herein, preferably refer to form translated by nucleic acid molecules, but are not limited to those composed of naturally-occurring amino acid only. The term may include those assembled into a composite of a plurality of polypeptide chains. The term also includes a naturally-occurring or an artificially modified amino acid polymer. Such modification includes disulfide bond formation, glycosylation, lipidation, acetylation, phosphorylation, or any other manipulation or modification (e.g., conjugation with a labeling moiety). This definition encompasses a peptide containing at least one amino acid analog (e.g., a nonnaturally-occurring amino acid), a peptide-like compound (e.g., peptoid), and other variants known in the art.

It should be understood that, as particularly mentioned herein, “protein” refers to an amino acid polymer having a relatively large molecular weight or a variant thereof, and “peptide” refers to an amino acid polymer having a relatively low molecular weight or a variant thereof.

In another embodiment, a chondrocyte capable of hypertrophication according to the present invention is derived from a mammal, preferably human, mouse, rat, or rabbit. There are two manner of ossification, the membranous ossification and chondral ossification. Membranous ossification is a manner which functions at the formation of flat bone (e.g., majority of cranial bones and clavicle) in nearby surface. In membranous ossification, membranous bone is directly formed without passing a chondrocyte intra connective tissue. The membranous ossification is also referred to as intramembraous ossification or connective tissue ossification. The chondral ossification is a manner which functions at the formation of endoskeleton (e.g., vertebra, costa, limb bone, and the like) in interior body. In chondral ossification, cartilages form first, the blood vessels infiltrate into the cadre to calcify the chondrocyte, and form calcified cartilages. The formation of calcified chondrocyte is crashed momentarily, and then osteogenesis and bone and primordial bone marrow occurs. In this conjuncture, after cartilage primordium is formed intra cartilage, the cartilage primordium is affected by the growth hormone and the like. Thereby a chondrocyte elongates and increases in size thereof towards long axis and minor axis. Thereafter, the blood vessels infiltrate in epiphysis to induce ossification. The chondral ossification is also endochondral ossification or enchondral ossification. (See, Fujita Hisao, Fujita Tsuneo, “hone no hassei” hyojun soshikigaku gairon, page 127 [“the development of bone” standard histological review, page 127]; kososhiki no kigen to shinka-josetsu-, Suda Tateo, The BONE, 18 kan, pages 421-426, 2004 [The origin and evolution of the hard tissue-introduction-, Suda Tateo, The BONE, 18th volume, pages 421-426, 2004; nainankotsu seikotsu keisei no katei, Suzuki Fujio, “hone wa donoyonishite dekiruka” Osaka Daigaku Shuppankai, page 21, 2004 [The process of endochondral ossification, Suzuki Fujio, “How does bone formation?” Osaka University Press, page 21, 2004]; Suzuki Takao et al. edit, “Hone no jiten”, Asakura shoten [Suzuki Takao et al. edit, “The dictionary of bone”, Asakura shoten]). Therefore, the chondrocyte capable of hypertrophication in the invention, which is capable of inducing the differentiation of undifferentiated cells into osteoblasts, exists evenly in the mammal including a rat, mouse, rabbit, human and the like. The agent plays play an important role in ossification. Thus, the agent of the present invention can be produced form the chondrocyte capable of hypertrophication by using the same procedure, in mammals and the like in which endochondral ossification occurs, in spite of species.

Using molecular biology methods, it was demonstrated that ossification is induced by implanting human recombinant BMP protein into rat, and BMP derived from human functions in the same manner as BMP protein derived from rat (See, Wozney, J. M. et al., Science, 242: 1528-1534, 1988. and Wuerzler K K. et al., J. Craniofacial Surg., 9: 131-137, 1998). It is also proven that the agent associated with ossification can be interchangeably used in between human and rat. It is known that these BMPs are different in the level of the amino acid sequences, but are substantively identical to the properties as protein (i.e., solid state properties on conditions of generation, and the like). The chondrocyte capable of hypertrophication according to the present invention may be isolated or induced from, for example, a region such as the chondro-osseous junction of costa, epiphysial line of long bone (e.g., femoris, tibia, fibula, humerus, ulna, and radius), epiphysial line of vertebra, zone of proliferating cartilage of ossicle (e.g., hand bones, foot bones and sterna), perichondrium, bone primordium formed from cartilage of fetus, the callus region of a healing bone-fracture and the cartilaginous part of bone proliferation phase. The chondrocyte capable of hypertrophication used in the present invention may be a chondrocyte obtained from any regions in which the chondrocyte can be capable of hypertrophication. The chondrocyte capable of hypertrophication can be obtained by inducing differentiation.

When an agent according to the present invention is produced by a chondrocyte, the chondrocytes may be typically adjusted to a cell density of 4×10⁴ cells/cm². The cell density is normally used in 10⁴ cells/cm² to 10⁶ cells/cm². However, cell densities of less than 10⁴ cells/cm² or more than 10⁶ cells/cm² may also be adjusted.

In the present invention, the culture of the chondrocyte capable of hypertrophication is prepared using cells isolated or induced by methods as described above.

The chondrocyte capable of hypertrophication used in the present invention may be cultured in any medium, which may include, but not limited to, Ham's F12 (HamF12), Dulbecco's Modified Eagle Medium (DMEM), Minimum Essential Medium (MEM), Minimum Essential Medium-alpha (alpha-MEM), Eagle's basal medium (BME), Fitton-Jackson Modified Medium (BGJb). The chondrocyte capable of hypertrophication may be cell cultured in a medium containing any substance which enhances the proliferation, differentiation or both of cells. In the present invention, the differentiation agent producing medium may comprise at least one conventional osteoblast differentiation component selected from the group consisting of glucocorticoid (e.g., dexamethasone, predonisolone, predonisone, cortisone, betamethasone, cortisol, corticosterone), β-glycerophosphate and ascorbic acid. The agent of the present invention is produced using the differentiation agent producing medium only comprising β-glycerophosphate and ascorbic acid. Preferably, the differentiation agent producing medium comprises all of glucocorticoid, β-glycerophosphate and ascorbic acid. In present invention, the differentiation agent producing medium may further comprise other components such as transforming growth factor-beta (TGF-beta), bone morphogenetic factor (BMP), leukemia inhibitory factor (LIF), colony stimulating factor (CSF), insulin-like growth factor (IGF), fibroblast growth factor (FGF), platelet-rich plasma (PRP), platelet-derived growth factor (PDGF), and vascular endothelial growth factor (VEGF). It may be useful that the differentiation agent producing medium further comprising a serum component (e.g., human serum, bovine serum, fetal bovine serum) is used.

The osteoblast produced by the agent capable of inducing differentiation of an osteoblast of the present invention can be implanted alone, or as composite materials with implants or bone repair materials into a subject, thereby making it possible for osteogenesis.

“Subject” as used herein refers to a biological organism to which a treatment according to the present invention is applied. It is also referred to as a “patient”. The subject or patient may be a dog, a cat or a horse, preferably a human.

An “implant” or a “bone repairing material” as used herein is utilized as the meaning generally used in the art. As used herein, they are substantially used in the same sense but, as particularly defined, an “implant” means all the materials used to fill and a “bone repairing material” means a material used to repair a defective region of bone.

A subcutaneous test for osteogenesis is a test for evaluating osteogenic function to generate bone in a region wherein bone does not originally exist, which is also referred to as prosthesis. Since this test can be performed easily, it is broadly used in the art. In the case of bone treatment, a bone deficit test can be used as a method of testing. Osteogenesis only occurs during this test under the conditions which readily promotes osteogenesis. Osteogenesis is performed by osteoblasts already existing in the immediate environment of a deficit and also those are induced/migrated thereto. Thus, it is normally believed that the rate of osteogenesis is better in the bone deficit test than in the subcutaneous test. It is well-known that the result of the subcutaneous test is consistent with the rate of osteogenesis in the actual bone deficit (see, e.g., Urist, M. R., Science, 150: 893-899 (1965), Wozney, J. M. et al., Science, 242: 1528-1532 (1988), Johnson, E. E. et al., Clin. Orthop., 230: 257-265 (1988), Ekelund, A. et al., Clin. Orthop., 263: 102-112 (1991), and Riley, E. H. et al., Clin. Orthop., 324: 39-46 (1996)). Therefore, if osteogenesis is observed as a result of the subcutaneous test, those skilled in the art understand that osteogenesis should also be induced in the bone deficit test.

(Composition Comprising an Agent Derived from a Chondrocyte Capable of Hypertrophication)

In one aspect, the present invention provides a composition comprising an agent obtainable by culturing a chondrocyte capable of hypertrophication in the differentiation agent producing medium. In another embodiment, the composition according to the present invention is used to induce the differentiation of the osteoblast, and is preferably used to induce differentiation of the undifferentiated cell into the osteoblast. The composition of the present invention may comprise at least one conventional osteoblast differentiation component selected from the group consisting of glucocorticoid, β-glycerophosphate and ascorbic acid. The composition of the present invention may comprise at least one conventional osteoblast differentiation component selected from the group consisting of β-glycerophosphate and ascorbic acid. In another embodiment, the composition of the present invention may comprise all of glucocorticoid, β-glycerophosphate and ascorbic acid as conventional osteoblast differentiation components. In another embodiment, the composition of the present invention may comprise both β-glycerophosphate and ascorbic acid as conventional osteoblast differentiation components.

(Composition Used for Production of an Agent Capable of Inducing the Differentiation of the Osteoblast)

In one aspect, The invention provides a composition used for production of an agent capable of inducing the differentiation of the osteoblast. The composition comprises a chondrocyte capable of hypertrophication. As used herein, the agent capable of inducing the differentiation of osteoblast may be used any other form above-described in (An agent capable of inducing the differentiation of osteoblast) and (Composition comprising an agent derived from a chondrocyte capable of hypertrophication) and the like in the present specification.

(Composition for Enhancing or Inducing Osteogenesis in a Biological Organism)

In one aspect, the present invention provides a composition for enhancing or inducing osteogenesis in a biological organism, wherein the composition comprises a chondrocyte capable of hypertrophication, which is capable of inducing the differentiation of osteoblasts. As used herein, the agent capable of inducing the differentiation of osteoblast may be any as described above in (An agent capable of inducing the differentiation of osteoblast) and (Composition comprising an agent derived from a chondrocyte capable of hypertrophication) and the like in the present specification.

(Kit for Producing an Agent Capable of Inducing the Differentiation of Osteoblast)

In one aspect, the present invention provides a kit for producing an agent capable of inducing the differentiation of osteoblast. The kit comprises A) composition used for production of an agent capable of inducing the differentiation; and B) conventional osteoblast differentiation component (at least one selected from the group consisting of glucocorticoid, β-glycerophosphate and ascorbic acid). As used herein, the agent capable of inducing the differentiation of osteoblast may be any as described above in (Composition used for production of an agent capable of inducing the differentiation of the osteoblast) and the like, in the present specification.

(Kit for Enhancing or Inducing Osteogenesis in a Biological Organism)

In one aspect, the present invention provides a kit for enhancing or inducing osteogenesis in a biological organism. The kit may comprise A) a chondrocyte capable of hypertrophication; and B) at least one conventional osteoblast differentiation component selected from the group consisting of glucocorticoid, β-glycerophosphate and ascorbic acid. As used herein, the agent capable of inducing the differentiation of osteoblast may be any as described above in (Composition used for production of an agent capable of inducing the differentiation of the osteoblast) and the like, in the present specification.

(Method of Production)

In one aspect, the present invention provides a method of producing a composition comprising an agent capable of inducing the differentiation of osteoblast. The method comprises culturing a chondrocyte capable of hypertrophication in a differentiation agent producing medium. The differentiation agent producing medium may comprise at least one conventional osteoblast differentiation component selected from the group consisting of glucocorticoid (e.g., dexamethasone, predonisolone, predonisone, cortisone, betamethasone, cortisol, corticosterone), β-glycerophosphate and ascorbic acid. The agent of the present invention is produced using the differentiation agent producing medium only comprising β-glycerophosphate and ascorbic acid. Preferably, the differentiation agent producing medium comprises all of glucocorticoid, β-glycerophosphate and ascorbic acid. More preferably, the differentiation agent producing medium comprises both β-glycerophosphate and ascorbic acid.

Glucocorticoid, β-glycerophosphate and ascorbic acid are known as conventional osteoblast differentiation components. However, it is known that their ability is limited. For example, it is known that glucocorticoid, β-glycerophosphate and ascorbic acid are not sufficient to have an effect on C3H10T1/2 cells, 3T3-Swiss albino cells, BALB/3T3 cells. Using the present method of production, the present invention successfully produces an agent capable of inducing the differentiation of osteoblast for a broad range of cells including conventional cell lines and/or cells distinct from conventional cells. Such an agent has not been known so far, therefore, it is considered that the existence of the agent itself is a significant effect.

In a preferred embodiment, the differentiation agent producing medium may further comprise a serum component (e.g., human serum, bovine serum, fetal bovine serum). Typically, the serum component may make up to 20% of the medium.

In another preferred embodiment, a chondrocyte capable of hypertrophication is derived from a mammal, preferably human, mouse, rat, or rabbit. The chondrocyte capable of hypertrophication may be a cell capable of hypertrophication which is induced by inducing differentiation. The chondrocyte capable of hypertrophication used in the present invention may be any chondrocyte which is capable of hypertrophication.

The medium used in the present invention may be any media in which the chondrocyte capable of hypertrophication can proliferate. For example, such medium includes, but not limited to: Ham's F12 (HamF12), Dulbecco's Modified Eagle Medium (DMEM), Minimum Essential Medium (MEM), Minimum Essential Medium-alpha (alpha-MEM), Eagle's basal medium (BME), Fitton-Jackson Modified Medium (BGJb).

In another preferred embodiment, the medium used in culturing the chondrocyte capable of hypertrophication in the present invention may contain any substance which enhances the proliferation, differentiation or both of the cells. The differentiation agent producing medium may comprise at least one conventional osteoblast differentiation component selected from the group consisting of glucocorticoid (e.g., dexamethasone, betamethasone, predonisolone, predonisone, cortisone, cortisol, corticosterone), β-glycerophosphate and ascorbic acid. The agent of the present invention is produced using the differentiation agent producing medium only comprising β-glycerophosphate and ascorbic acid. Preferably, the differentiation agent producing medium comprises all of glucocorticoid, β-glycerophosphate and ascorbic acid. More preferably, the differentiation agent producing medium comprises both β-glycerophosphate and ascorbic acid. In present invention, the differentiation agent producing medium may further comprise other components such as transforming growth factor-beta (TGF-beta), bone morphogenetic factor (BMP), leukemia inhibitory factor (LIF), colony stimulating factor (CSF), insulin-like growth factor (IGF), fibroblast growth factor (FGF), platelet-rich plasma (PRP), platelet-derived growth factor (PDGF), and vascular endothelial growth factor (VEGF). It may be useful that the differentiation agent producing medium further comprises a serum component (e.g., human serum, bovine serum, fetal bovine serum).

In another embodiment, the chondrocyte capable of hypertrophication may be obtained from any region including the chondro-osseous junction of costa, epiphysial line of long bone (e.g., femoris, tibia, fibula, humerus, ulna, and radius), epiphysial line of vertebra, zone of proliferating cartilage of ossicle (e.g., hand bones, foot bones and sterna), perichondrium, bone primordium formed from cartilage of fetus, the callus region of a healing bone-fracture or the cartilaginous part of bone proliferation phase. The chondrocyte capable of a hypertrophication used in the present invention may be chondrocyte obtained from any regions in which the chondrocyte is capable of hypertrophication.

In other preferred embodiment, the agent capable of inducing differentiation of an osteoblast according to the present invention may be obtained by only the step of harvesting a supernatant of the differentiation agent producing medium, wherein the chondrocyte capable of hypertrophication is cultured. Preferably, the agent capable of inducing differentiation of an osteoblast may be further extracted from the supernatant harvested. The agent capable of inducing differentiation of an osteoblast may be secreted from a chondrocyte capable of hypertrophication, and may exist within a chondrocyte capable of hypertrophication.

In the present specification, the period for culturing the chondrocyte capable of hypertrophication may be a period that the agents of quantum sufficit are produced (e.g., several months to half year, or 3 days to 3 weeks (e.g., 3 days, 4 days, 5 days, 6 days, 7 days, 8 days, 9 days, 10 days, 20 days, more than 1 month, half year, 5 months, 4 months, 3 months, 2 months, 1 month, less than 3 weeks and possible combinations thereof within any range). When the period of culture is lengthened, and cells are confluent in a culture vessel, it is preferable to passage the cells.

(A Method of Producing an Agent Capable of Inducing the Differentiation of Osteoblast)

In one aspect, the present invention provides a method of producing an agent capable of inducing the differentiation of osteoblast. The method comprises culturing a chondrocyte capable of hypertrophication in a differentiation agent producing medium, wherein the differentiation agent producing medium may comprise at least one conventional osteoblast differentiation component selected from the group consisting of glucocorticoid, β-glycerophosphate and ascorbic acid. As used herein, the agent capable of inducing the differentiation of osteoblast may be any as described above in (An agent capable of inducing the differentiation of osteoblast), (Composition comprising an agent derived from a chondrocyte capable of hypertrophication) and (Method of production) and the like in the present specification.

(Method of Inducing the Differentiation of Undifferentiated Cell into Osteoblast)

In another aspect, the present invention provides the method of producing the osteoblast by inducing the differentiation of an undifferentiated cell into an osteoblast. The method comprising A) inoculating the an undifferentiated cell to a culture scaffold or a culture vessel; and B) exposing the undifferentiated cell to an agent produced by a chondrocyte capable of hypertrophication by adding a solution including the agent capable of inducing the differentiation of osteoblast to the medium or exchanging the medium for a medium including the agent, after the undifferentiated cell is stabilized. The undifferentiated cell and the agent capable of inducing the differentiation of osteoblast which are used in the method of inducing the differentiation of an undifferentiated cell into an osteoblast may be any as described above in (An agent capable of inducing the differentiation of osteoblast) and (Method of production) and the like in the present specification.

According to the present specification, the undifferentiated cell being “stabilized” means that the cell recovers the natural state, including the state recovered from injury due to an enzyme upon separation or the state that an adhesive cell attaches a vessel and the like.

(Osteoblast)

In another aspect, the present invention provides an osteoblast produced by contacting an agent derived from a chondrocyte capable of hypertrophication with an undifferentiated cells. In the production of osteoblast, any that is described above in (An agent capable of inducing the differentiation of osteoblast) and the like in the present specification may be used. The osteoblast produced can be used in the same manner as a natural osteoblast. Therefore, the osteoblast in the present invention can be used in the therapy for bone deficits and the like, and for the production of the implant alone, a composite material together with scaffold, or the like.

(Composite Material for Producing an Agent Capable of Inducing the Differentiation of Osteoblast)

In one aspect, the invention provides a composite material for producing an agent capable of inducing the differentiation of osteoblast. The composite material may comprise A) a chondrocyte capable of hypertrophication; and B) a scaffold. In one embodiment, the scaffold includes, but not limited to, a material selected from the group consisting of calcium phosphate, calcium carbonate, alumina, zirconia, apatite-wollastonite deposited glass, gelatin, collagen, chitin, fibrin, hyaluronic acid, extracellular matrix mixture (e.g., Matrigel™), silk, cellulose, dextran, agarose, agar, synthetic polypeptide (e.g., PuraMatrix™), polylactic acid, polyleucine, alginic acid, polyglycolic acid, polymethyl methacrylate, polycyanoacrylate, polyacrylonitrile, polyurethan, polypropylene, polyethylene, polyvinyl chloride, ethylene-vinyl acetate copolymer, nylon and a combination thereof. Preferably, the scaffold can comprise of hydroxyapatite. As used herein, the agent capable of inducing the differentiation of osteoblast may be any as described above in (An agent capable of inducing the differentiation of osteoblast) and (Composition comprising an agent derived from a chondrocyte capable of hypertrophication) and the like in the present specification.

In another aspect, the present invention provides the kit for producing an agent capable of inducing the differentiation of osteoblast comprising: A) the composite material for producing an agent capable of inducing the differentiation of osteoblast; and B) a conventional osteoblast differentiation component (at least one selected from the group consisting of glucocorticoid, β-glycerophosphate and ascorbic acid). As used herein, the agent capable of inducing the differentiation of osteoblast may be any described as above in (Composite material) and the like in the present specification.

(Composite Material for Enhancing or Inducing Osteogenesis in a Biological Organism)

In one aspect, the present invention provides a composite material for enhancing or inducing osteogenesis in a biological organism. The composite material may comprise A) a chondrocyte capable of hypertrophication, which is capable of inducing the differentiation of osteoblasts; and B) a scaffold that is biocompatible with the biological organism. In one embodiment, the scaffold includes, but not limited to a material selected from the group consisting of calcium phosphate, calcium carbonate, alumina, zirconia, apatite-wollastonite deposited glass, gelatin, collagen, chitin, fibrin, hyaluronic acid, extracellular matrix mixture (e.g., Matrigel™), silk, cellulose, dextran, agarose, agar, synthetic polypeptide (e.g., PuraMatrix™), polylactic acid, polyleucine, alginic acid, polyglycolic acid, polymethyl methacrylate, polycyanoacrylate, polyacrylonitrile, polyurethan, polypropylene, polyethylene, polyvinyl chloride, ethylene-vinyl acetate copolymer, nylon and a combination thereof. Preferably, the scaffold can comprise of hydroxyapatite. As used herein, the agent capable of inducing the differentiation of osteoblast may be any as described above in (An agent capable of inducing the differentiation of osteoblast) and (Composition comprising an agent derived from a chondrocyte capable of hypertrophication) and the like in the present specification.

“Composite material” according to the present specification refers to a material comprising a cell and a scaffold.

“Enhancing” osteogenesis as used herein refers to increasing the rate of osteogenesis at a site where osteogenesis has already occurred. As used herein, “Inducing” osteogenesis refers to causing osteogenesis at a site where the osteogenesis has not occurred.

(Scaffold)

A “scaffold” according to the present specification refers to a material to support cells. The scaffold has constant strength and biocompatibility. As used herein, the scaffold is produced from biological materials, naturally supplied materials, or naturally occurring materials or synthetically supplied materials. As used herein, the scaffold is formed from materials other than organisms such as tissues or cells (i.e., non-cellular material). As used herein, the scaffold is a composition formed from materials other than organisms such as tissues or cells, including materials derived from living organisms such as collagen or hydroxyapatite. As used herein, an “organism” refers a material-system organized to have a living function. That is, the term “organism” distinguishes living beings from other material-systems. The concept of the organism comprises cells, tissues or others, while materials derived from living being, extracted from the organism, are not included in the organism. The scaffold region to which cells are fixed to includes a surface of the scaffold, or an internal pore of the scaffold if it has such internal pore that can contain cells. For example, a scaffold made from hydroxyapatite includes many pores which can normally contain cells sufficiently.

The material for the scaffold includes, but not limited to a material selected from the group consisting of calcium phosphate, calcium carbonate, alumina, zirconia, apatite-wollastonite deposited glass, gelatin, collagen, chitin, fibrin, hyaluronic acid, extracellular matrix mixture (e.g., Matrigel™), silk, cellulose, dextran, agarose, agar, synthetic polypeptide (e.g., PuraMatrix™), polylactic acid, polyleucine, alginic acid, polyglycolic acid, polymethyl methacrylate, polycyanoacrylate, polyacrylonitrile, polyurethan, polypropylene, polyethylene, polyvinyl chloride, ethylene-vinyl acetate copolymer, nylon and combinations thereof. Preferably, the scaffold materials are calcium phosphate, gelatin, or collagen. More preferably, the scaffold material is hydroxyapatite.

These scaffolds may be provided in any form such as a granular form, a block form, or a sponge form. This scaffold may be porous or non-porous. For such scaffolds, those commercially available (e.g., from PENTAX Corporation, OLYMPUS Corporation, Kyocera Corporation, Mitsubishi Pharma Corporation, Dainippon Sumitomo Pharmaceuticals, Kobayashi Pharmaceuticals Co. Ltd., Zimmer Inc.) can be used. Standard procedures for preparation and characterization of scaffolds are known in the art, which only require routine experimentation and techniques commonly known in the art. For example, see U.S. Pat. No. 4,975,526; No. 5,011,691; No. 5,171,574; No. 5,266,683; No. 5,354,557; and No. 5,468,845, which are incorporated herein as references. Other scaffolds are also described, for example, in the following documents: articles for biocompatible materials, such as LeGeros and Daculsi, Handbook of Bioactive Ceramics, II pp. 17-28 (1990, CRC Press); other published descriptions, such as Yang Cao, Jie Weng, Biomaterials 17 (1996) pp. 419-424; LeGeros, Adv. Dent. Res. 2, 164 (1988); Johnson et al., J. Orthopaedic Research, 1996, vol. 14, pp. 351-369; and Piattelli et al., Biomaterials 1996, vol. 17, pp. 1767-1770, the disclosures of which are herein incorporated as references.

“Calcium phosphate” described herein is the generic name for phosphates of calcium, which include, but not limited to compounds represented by the following chemical formulas: CaHPO₄, Ca₃(PO₄)₂, Ca₄O(PO₄)₂, Ca₁₀(PO₄)₆(OH)₂, CaP₄O₁₁, Ca(PO₃)₂, Ca₂P₂O₇, or Ca(H₂PO₄)₂.H₂O.

“Hydroxyapatite” described herein refers to a compound whose general composition is Ca₁₀(PO₄)₆(OH)₂, which is a main component of mammalian hard tissues (bone and teeth), like collagen. Although hydroxyapatite contains a series of calcium phosphates as described above, the PO₄ and OH components within the apatite in the hard tissues of biological organisms are often substituted with a CO₃ component in body fluids. Furthermore, hydroxyapatite is a material having safety approval by the Ministry of Health, Labour and Welfare of Japan, and the FDA (U.S. Food and Drug Administration). Although many commercially available hydroxyapatites are non-absorbable by the body and remain hardly absorbed in the body, some are absorbable.

“Extracellular matrix mixture” in the present specification refers to a mixture of extracellular matrix and growth factor. The extracellular matrix includes, but not limited to, laminin, collagen and the like. The extracellular matrix may be derived from a biological organism or synthesized.

“Bone deficit” as described herein comprises, but not limited to: lesions such as bone tumors, osteoporosis, rheumatoid arthritis, osteoarthritis, osteomyelitis, and osteonecrosis; correction such as immobilization of the bone, foraminotomy and osteotomy; trauma such as complex fracture; and bone deficits derived from collecting ilium.

The osteoblast produced by the agent according to the present invention can be used in the production of extracellular matrix, or the repair and reconstitution of bone. The region implanted includes, but not limited to bone deficits due to the lesion and excision of bone tumor and the like, which are normally desired for repair and reconstitution of bone. The implantation can be conducted in the same manner as known implantation using bone stem cells. The number of the cells implanted is properly selected according to the size of bone deficit and symptoms and the like, and is normally suitable with 10⁴-10⁸ cells. The osteoblast produced by the agent according to the present invention can also be cultured in the tissue culture vessel and proliferated up to a sufficient amount for the use of implantation. Furthermore, the osteoblast produced by the agent according to the present invention can also be attached to a suitable carrier in vitro to enhance the proliferation of a cell and to produce the extracelluar matrix.

The present invention can use optionally together with physiologically active substance such as cytokine.

“Cellular physiologically active substance” or “physiologically active substance” is interchangeably used herein to refer to a substance which affects cells or tissues. Such effects comprise, for example, but not limited to, the control or modification of the cells or tissues. The physiologically active substance includes cytokines or growth factors. The physiologically active substance may be naturally occurring or a synthesized substance. Preferably, the physiologically active substance is produced in a cell. It also includes substances produced in a cell, or substances having a function similar to, but modified from, those produced in a cell. In the present specification, the physiologically active substance may be in the form of proteins including peptides, in the form of nucleic acids, or in other forms.

“Cytokine” used herein is defined as having a similar meaning to that used in the art in the broadest sense. It refers to a physiologically active substance produced in a cell that affects the same or a different cell. Generally, a cytokine is a protein or polypeptide and has activities that control the immune response, modulate the endocrine system, modulate the nervous system, affect anti-tumor action, affect anti-viral action, modulate cell growth, modulate cell differentiation, modulate cellular function and others. In the present specification, cytokines may be proteins, nucleic acids, or in other forms. However at the time of actually affecting cells, cytokines are often proteins, including peptides.

“Growth factor” or “cellular growth factor” used herein interchangeably refers to a substance which enhances or controls the induction of the growth and differentiation of cells. Growth factor is also a proliferation or development factor. In cell culture or tissue culture, growth factors can be added to the medium and substituted for the function of macromolecules in the serum. It is proved that, in addition to cell growth, many growth factors function as factors that regulate differentiation.

Cytokines associated with osteogenesis typically include factors such as transforming growth factor-beta (TGF-beta), bone morphogenetic factor (BMP), leukemia inhibitory factor. (LIF), colony stimulating factor (CSF), insulin-like growth factor (IGF), fibroblast growth factor (FGF), platelet-rich plasma (PRP), platelet-derived growth factor (PDGF), and vascular endothelial growth factor (VEGF); and compounds such as ascorbic acid, glucocorticoid, and glycerophosphoric acid.

Since physiologically active substances such as cytokines and growth factors are generally redundant, cytokines or growth factors known by another name and function (such as cell adhesion activity or cell-matrix adhesion activity) can be also used in the present invention, as long as they have the activity of the physiologically active substance to be used in the invention. Cytokines or growth factors can be used in the implementation of the invention, as long as they have preferred activity (such as stem cell growth activity or osteoblast differentiation activity, the activity of promoting a chondrocyte capable of hypertrophication to produce the agent of the present invention) for the present invention.

The agent of the present invention may be derived from a cell derived from a syngenic or an allogenic individual, or derived from a heterologous individual.

“Derived from syngenic” as used herein means derived from an autologous, pure line, or inbred line.

“Derived from an allogenic individual” as used herein means derived from another individual of the same species that is genetically different.

“Derived from a heterologous individual” as used herein means derived from a heterologous individual. Thus, for example, when the recipient is human, cells from rat are “deriving from an individual who is heterologous in relation to a biological organism”.

(Use in the Production of an Agent Capable of Inducing the Differentiation of Osteoblast)

In one aspect, the present invention provides a use of a chondrocyte capable of hypertrophication for the production of an agent capable of inducing the differentiation of osteoblast. In another aspect, the present invention provides a use of a chondrocyte capable of hypertrophication and the conventional osteoblast differentiation component for the production of an agent capable of inducing the differentiation of osteoblast. In the uses, the agent capable of inducing the differentiation of osteoblast may be any as described above in (An agent capable of inducing the differentiation of osteoblast) and (Composition comprising an agent derived from a chondrocyte capable of hypertrophication) and the like in the present specification.

(Use in the Manufacture of an Implant or a Bone Repairing Material for Enhancing or Inducing Osteogenesis in a Biological Organism)

In one aspect, the present invention provides a use of: A) a chondrocyte capable of hypertrophication, which is capable of inducing the differentiation of osteoblasts; and B) a scaffold that is biocompatible with the biological organism, in the manufacture of an implant or a bone repairing material for enhancing or inducing osteogenesis in a biological organism. The scaffold may be, but not limited to, a material selected from the group consisting of calcium phosphate, calcium carbonate, alumina, zirconia, apatite-wollastonite deposited glass, gelatin, collagen, chitin, fibrin, hyaluronic acid, extracellular matrix mixture (e.g., Matrigel™), silk, cellulose, dextran, agarose, agar, synthetic polypeptide (e.g., PuraMatrix™), polylactic acid, polyleucine, alginic acid, polyglycolic acid, polymethyl methacrylate, polycyanoacrylate, polyacrylonitrile, polyurethan, polypropylene, polyethylene, polyvinyl chloride, ethylene-vinyl acetate copolymer, nylon and a combination thereof. Preferably, the scaffold can comprise of hydroxyapatite. In the uses, the agent capable of inducing the differentiation of osteoblast may be any as described above in (An agent capable of inducing the differentiation of osteoblast) and the like in the present specification.

(Method for Enhancing or Inducing Osteogenesis in a Biological Organism)

In one aspect, the present invention provides a method for enhancing or inducing osteogenesis in a biological organism. The methods may comprise locating a composite material in a region in need thereof, wherein the composite material comprises a chondrocyte capable of hypertrophication, which is capable of inducing the differentiation of osteoblasts and a scaffold that is biocompatible with the biological organism. The scaffold may be, but not limited to, a material selected from the group consisting of calcium phosphate, calcium carbonate, alumina, zirconia, apatite-wollastonite deposited glass, gelatin, collagen, chitin, fibrin, hyaluronic acid, extracellular matrix mixture (e.g., Matrigel™), silk, cellulose, dextran, agarose, agar, synthetic polypeptide (e.g., PuraMatrix™), polylactic acid, polyleucine, alginic acid, polyglycolic acid, polymethyl methacrylate, polycyanoacrylate, polyacrylonitrile, polyurethan, polypropylene, polyethylene, polyvinyl chloride, ethylene-vinyl acetate copolymer, nylon and a combination thereof. Preferably, the scaffold can comprise of hydroxyapatite. In the use, the agent capable of inducing the differentiation of osteoblast may be any as described above in (An agent capable of inducing the differentiation of osteoblast) and the like in the present specification.

Hereinafter, the present invention will be described by various examples. The Examples described below are provided only for illustrative purposes. Accordingly, the scope of the present invention is not limited by the above-described embodiments or the examples below, and instead is limited only by the appended claims.

### EXAMPLES

### Example 1

### Preparation and Detection of a Cellular Function Regulating Agent Produced by Culturing a Chondrocyte Capable of Hypertrophication from Costa/Costal Cartilages in the MEM Differentiation Agent Producing Medium

(Preparation of a Chondrocyte Capable of Hypertrophication from Costa/Costal Cartilages)

Four week-old male rats (Wistar) group and 8 week-old male rats (Wistar) group were, respectively, examined in the present Example. These rats were sacrificed using chloroform. The rats' chests were shaved using a razor and their whole bodies were immersed in Hibitane (10-fold dilution) to be disinfected. The rats' chests were incised and the costa/costal cartilages removed aseptically. The translucent growth cartilage region was collected from the boundary region of the costa/costal cartilages. The growth cartilage was sectioned and incubated in 0.25% trypsin-EDTA/Dulbecco's phosphate buffered saline (D-PBS) at 37° C. for 1 hour, with stirring. The sections were then washed and collected by centrifugation (170×g for 3 min.) and followed by incubation in 0.2% Collagenase (Invitrogen)/D-PBS at 37° C. for 2.5 hours, with stirring. After collection by centrifugation (170×g for 3 min.), the cells were incubated in 0.2% Dispase (Invitrogen)/(HAM+10% FBS) in a stirring flask overnight at 37° C. with stirring. In the following day, the resulting cell suspension was filtered and the cells washed and collected by centrifugation (170×g for 3 min.). The cells were stained with trypan blue and counted under a microscope.

The cells were evaluated as cells not stained were considered to be living cells, and those stained blue were considered to be dead cells.

(Identification of a Chondrocyte Capable of Hypertrophication)

Since the cells obtained in Example 1 were impaired by the enzymes used in cell separation (e.g., trypsin, collagenase, and dispase), they were cultured to recover. Chondrocytes capable of hypertrophication were identified by using their expression of chondrocyte markers and their morphological hypertrophy under a microscope.

(Localization or Expression of Specific Markers for a Chondrocyte Capable of Hypertrophication)

A cell suspension prepared using a method as described above is treated with sodium dodecyl sulfate (SDS). The SDS-treated solution is subjected to SDS polyacrylamide gel electrophoresis. The gel is blotted onto a transfer membrane (Western blotting), reacted with a primary antibody to a chondrocyte marker, and detected with a secondary antibody labeled with an enzyme such as peroxidase, alkaline phosphatase or glucosidase, or a fluorescent tag such as fluorescein isothiocyanate (FITC), phycoerythrin (PE), Texas Red, 7-amino-4-methylcoumarin-3-acetate (AMCA) or rhodamine.

Cell cultures prepared using a method as described above are fixed with 10% neutral formalin buffer, reacted with a primary antibody to a chondrocyte marker, and detected with a secondary antibody labeled with an enzyme such as peroxidase, alkaline phosphatase or glucosidase, or a fluorescent tag such as FITC, PE, Texas Red, AMCA or rhodamine.

The alkaline phosphatase can be detected by staining. A cell culture obtained by the above-described manipulation was fixed with 60% acetone/citric acid buffer, washed with distilled water, and soaked in the mixture of First Violet B and Naphthol AS-MX at RT in the dark for 30 minutes to react, and thereby stained.

(Histological Assessment of the Ability of Hypertrophication in Chondrocytes)

5×10⁵ cells in a HAM's F12 medium were centrifuged to prepare a pellet of cells. The pellet was cultured for a pre-determined period. Cell sizes before and after culture were compared under a microscope. When a significant increase in size was observed, the cells were determined to be capable of hypertrophication.

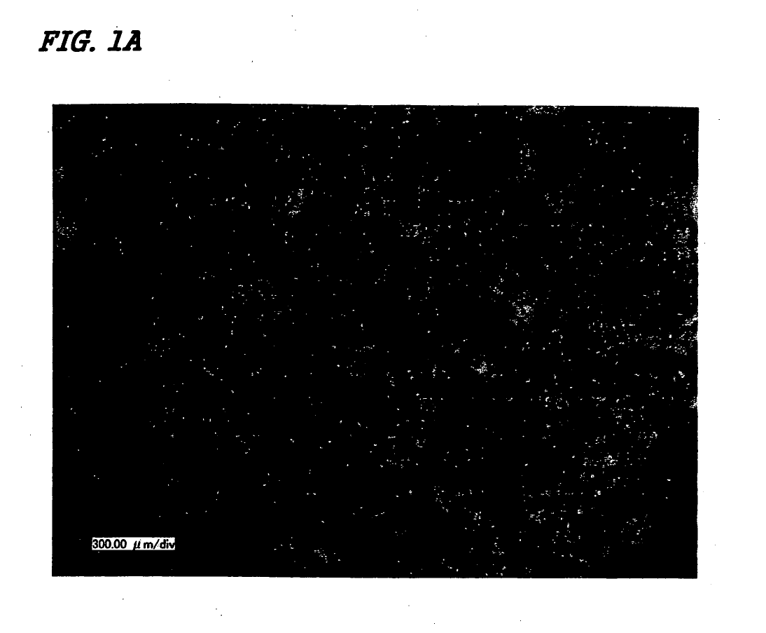

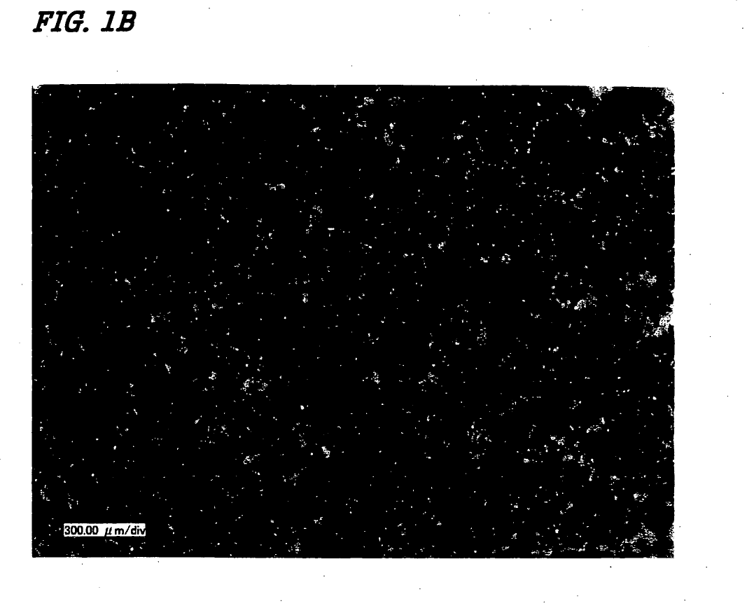

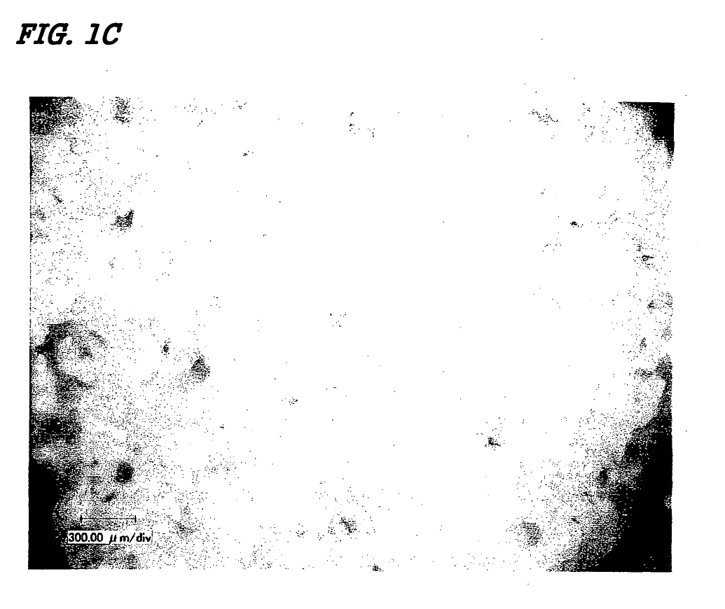

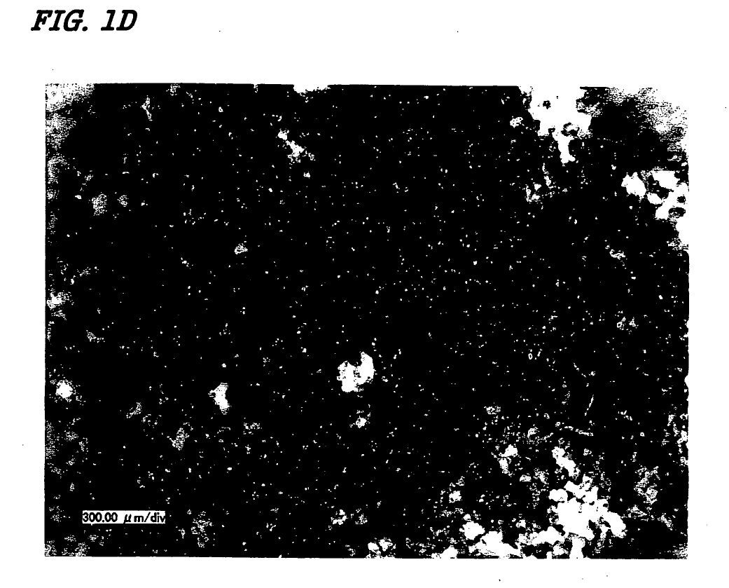

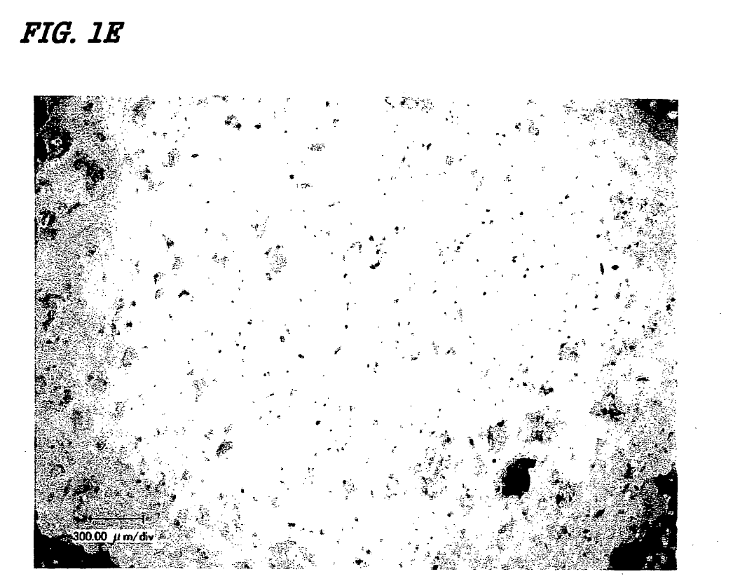

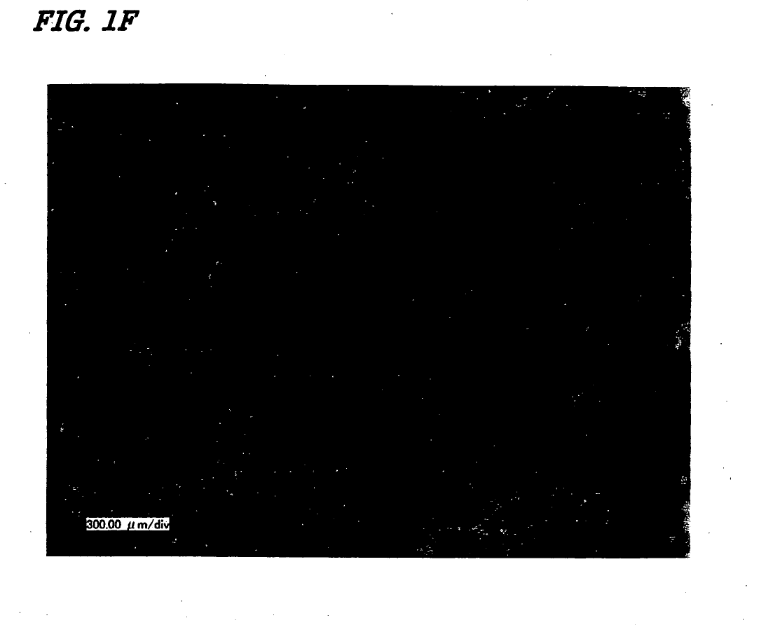

To determine whether chondrocytes capable of hypertrophication were present in cell suspensions in which chondrocytes capable of hypertrophication were diluted, the following experiment was performed. A density of 1×10⁶ cells/ml of chondrocytes capable of hypertrophication were inoculated to hydroxyapatite, and incubated in a 5% CO₂ incubator at 37° C. for one week. The sample (hydroxyapatite inoculated with cells) were then stained with alkaline phosphatase or stained with toluidine blue. For alkaline phosphatase staining, the sample was fixed by immersing in 60% acetone/citric acid buffer for 30 second, rinsed in water, and incubated with an alkaline phosphatase staining solution (2 ml of 0.25% naphthol AS-MX alkaline phosphate (Sigma-Aldrich)+48 ml 25% First Violet B salt solution (Sigma-Ardrich)) at RT in the dark for 30 minutes. For toluidine blue staining, the sample was incubated with a toluidine blue staining solution (0.25% toluidine blue solution, pH 7.0, Wako Pure Chemical Industries Ltd.) at RT for 5 min. The sample displayed red spotted staining with alkaline phosphate (See FIG. 1A). With toluidine blue, the same site of the sample displayed blue spotted staining, showing the presence of cells (See FIG. 1B). Thus, it was observed that cells in hydroxyapatite have an alkaline phosphatase activity.

(Results)

The cells obtained in Example 1 expressed a chondrocyte marker, and were determined to be morphologically-hypertrophic. This shows that the cells obtained in Example 1 were chondrocytes capable of hypertrophication. These cells were used in the following experiments.

(Detection of the Agent Produced by a Chondrocyte Capable of Hypertrophication Collected from the Costa/Costal Cartilage)

Chondrocytes capable of hypertrophication obtained in Example 1 were diluted to 4×10⁴ cell/cm² in an MEM differentiation agent producing medium (Minimum Essential Medium (MEM), with a final of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was inoculated evenly to the dish (Becton Dickinsin), and cultured in a 5% CO₂ incubator at 37° C., followed by a time course (4 day, 7 day, 11 day, 14 day, 18 day, 21 day) collecting supernatants of the medium.

(Studies on Whether the Culture Supernatant Collected is Capable of Inducing the Differentiation of an Undifferentiated Cell into an Osteoblast)

A density of 1.25×10⁴ cell/cm² of mouse C3H10T1/2 cells (Dainippon Sumitomo Pharmaceutical, CCL-226) were inoculated evenly in 24-well plates (Becton Dickinson, 2.5×10⁴/well). Eighteen hours after inoculation, 1 ml of the culture supernatant was added to the plates and cultured in a 5% CO₂ incubator at 37° C. After 72 hours, alkaline phosphatase activity was measured using the following procedures.

(Measurement of an Alkaline Phosphatase Activity)

To measure an alkaline phosphatase activity, 100 μl of the samples with or without the agent were, respectively, mixed with a solution (50 μl) comprising 4 mg/ml p-nitrophenyl phosphate and 50 μl an alkali buffer (Sigma, A9226), and reacted at 37° C. for 15 minutes. Thereafter, the reaction was terminated by adding 50 μl 1N NaOH to the samples, and the absorbances (at 405 nm) were determined thereof. Then, 20 μl concentrated hydrochloric acid were further added the samples to determinate the absorbances (at 405 nm). The difference of these absorbancies was referred to as “absolute active value” (indicated as “absolute value” in the table), and was used as a indicator of the alkaline phosphatase activity. In the 4 week-old group, five experiments were performed and three trials were carried out per experiment. In the 8 week-old group, three experiments were performed and two trials in the first experiment, two trials in the second experiment, and one trial in the third experiment were performed. The absolute value of each sample divided by the absolute value of the medium only control (absolute active value which was determined using the medium as a control only added to mouse C3H10T1/2 cells in the same manner) was referred to “relative active value” (indicated as “relative value” in the table) in the present specification. The relative active value was used as another indicator of the alkaline phosphatase activity. In the present Example, the agent was determined to be capable of increasing a value of alkaline phosphatase (ALP) activity when the agent was capable to increase the value of alkaline phosphatase activity of a mouse C3H10T1/2 cell by more than 1.5 times comparing that of the cells cultured in the medium with and without the agent of the present invention.

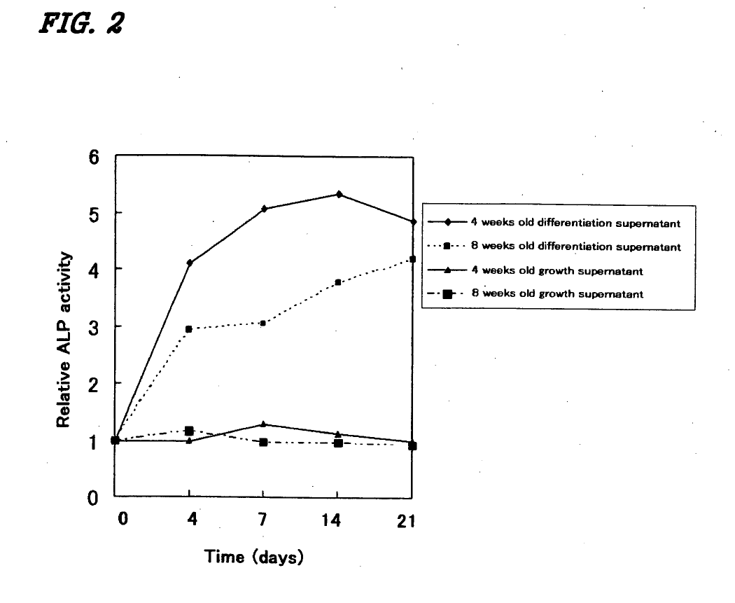

To evaluate the alkaline phosphatase activity using the relative active value, the value of the alkaline phosphatase activity of a sample added with only MEM differentiation agent producing medium was defined as 1. In the group of 4 week-old rats, the relative active value increased about 4.1 times when a culture supernatant collected after 4 days was added, to about 5.1 times when a culture supernatant collected after 1 week was added, to about 5.4 times when a culture supernatant collected after 2 weeks was added, and to about 4.9 times when a culture supernatant collected after 3 weeks was added. In the group of 8 week-old rats, the relative active value increased to about 2.9 times when a culture supernatant collected after 4 days was added, to about 3.1 times when a culture supernatant collected after 1 week was added, to about 3.8 times when a culture supernatant collected after 2 weeks was added, and to about 4.2 times when a culture supernatant collected after 3 weeks was added (see Table 1, upper column, and FIG. 2).

(Identification of an Osteoblast)

(Staining with Alkaline Phosphatase)

(In the Case of Chondrocytes Capable of Hypertrophication were Added to an MEM Differentiation Agent Producing Medium)

A density of 1.25×10⁴ cells/cm² (i.e., 2.5×10⁴/well) of mouse C3H10T1/2 cells (Dainippon Sumitomo Pharmaceutical, CCL-226) were inoculated evenly in 24-well plates (Becton Dickinson). A density of 1×10⁶ cells/ml of mouse C3H10T1/2 cells were inoculated evenly in hydroxyapatites. Eighteen hours after inoculation, the plates and hydroxyapatites were added with the culture supernatant (1 ml) cultured with chondrocytes capable of hypertrophication in an MEM differentiation agent producing medium, and cultured in a 5% CO₂ incubator at 37° C. The cell culture was fixed with 60% acetone/citric acid buffer, washed with distilled water, and soaked in the mixture of First Violet B and Naphthol AS-MX at RT in the dark for 30 minutes to react, and thereby stained.

(In the Case of Chondrocytes Capable of Hypertrophication were Added to an MEM Growth Medium)

A density of 1.25×10⁴ cells/cm² (i.e., 2.5×10⁴/well) of mouse C3H10T1/2 cells (Dainippon Sumitomo Pharmaceutical, CCL-226) were inoculated evenly in 24-well plates (Becton Dickinson). A density of 1×10⁶ cells/ml of mouse C3H10T1/2 cells were inoculated evenly in hydroxyapatites. Eighteen hours after inoculation, the plates and hydroxyapatites were added with the culture supernatant (1 ml) cultured with chondrocytes capable of hypertrophication in an MEM growth medium, and cultured in a 5% CO₂ incubator at 37° C. The cell culture was fixed with 60% acetone/citric acid buffer, washed with distilled water, and soaked in the mixture of First Violet B and Naphthol AS-MX at RT in the dark for 30 minutes to react, and thereby stained.

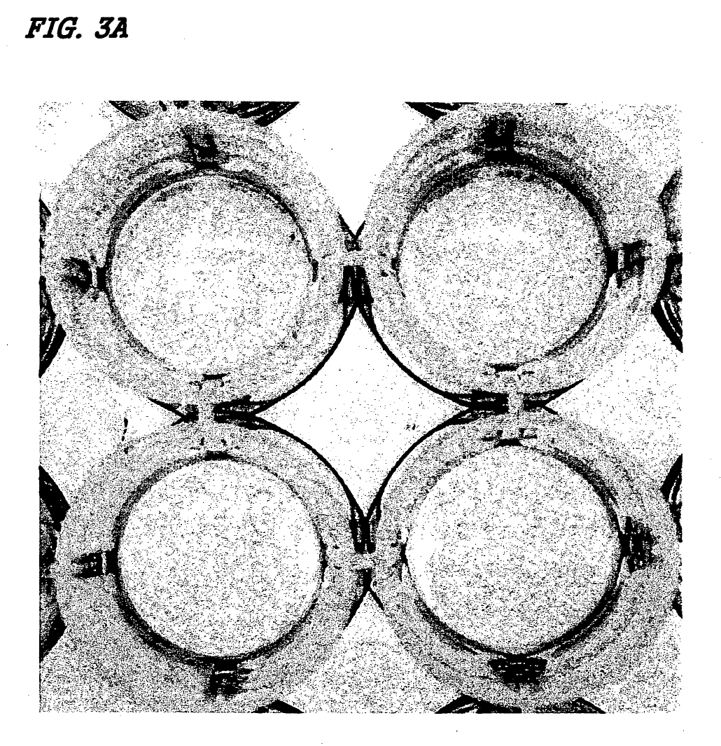

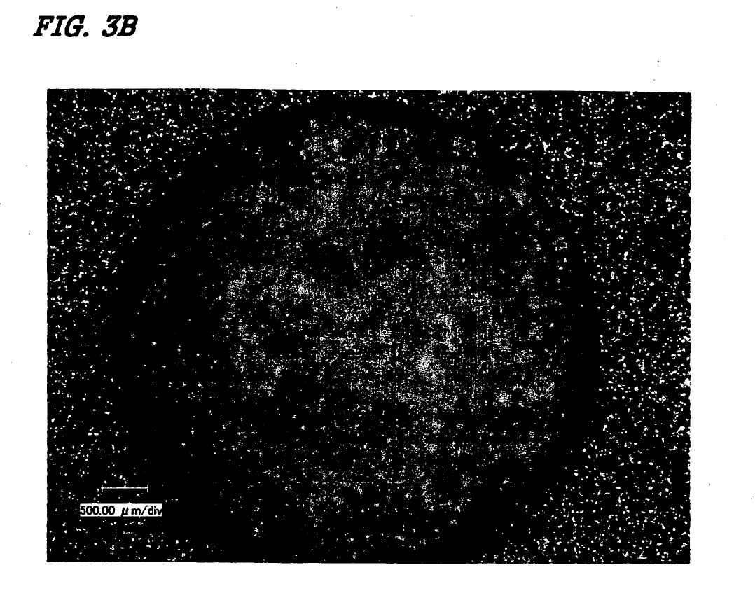

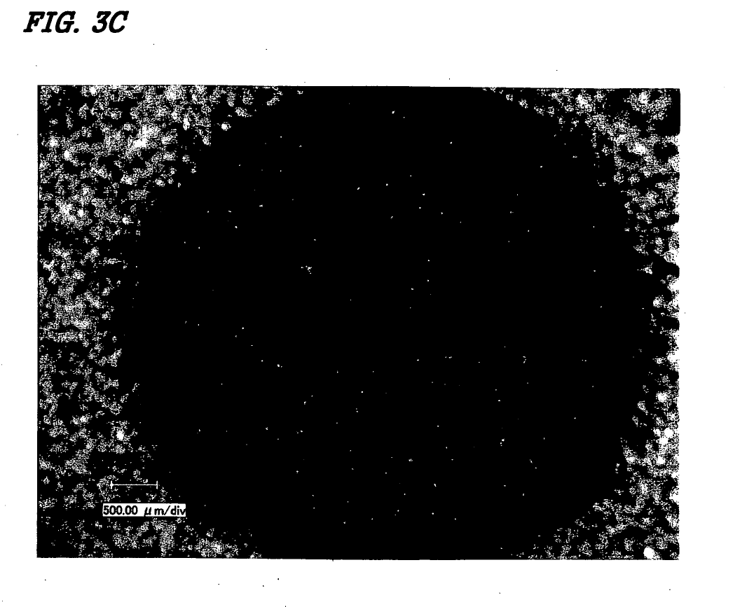

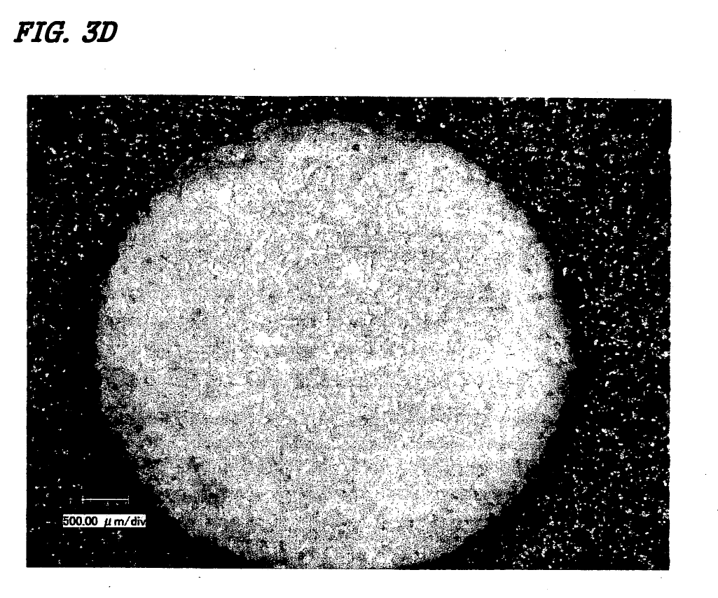

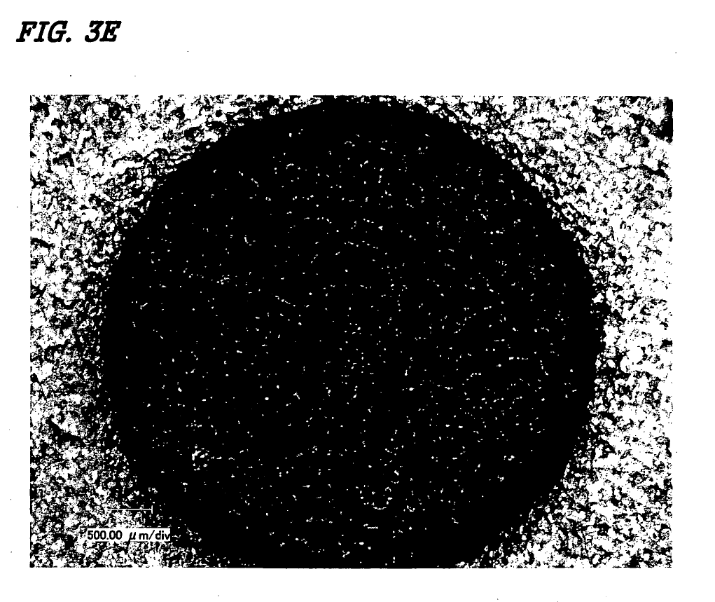

As described above, it was shown that the alkaline phosphatase (ALP) activity, which is one of the osteoblast markers, of mouse C3H10T1/2 cells was increased by an agent capable of inducing the differentiation into osteoblasts. Furthermore, it was also shown that C3H10T1/2 cells were stained red after adding the agent capable of inducing the differentiation into osteobalasts in the alkaline phosphatase staining of C3H10T1/2 cell. Therefore, the expression of alkaline phosphatase was indicated using the staining method. As a result, it was confirmed that C3H10T1/2 cells were differentiated into osteoblasts (see Table 1, upper column, FIG. 2, FIG. 3A, upper column, and FIG. 3B). Furthermore, the pellet of the cells was prepared by the above-described method, and stained with acid toluidine blue and safranine O. As a result, no metachromasia was shown and safranine staining was negative. Thus, it was confirmed that the cells are not chondrocytes. Therefore, it could be confirmed that the differentiated cells was not chondrocytes capable of hypertrophication.

### Comparative Example 1A

### Preparation and Detection of the Agent Produced by Culturing a Chondrocyte Capable of Hypertrophication Derived from Costa/Costal Cartilage in an MEM Growth Medium)

Chondrocytes capable of hypertrophication were collected from the costa/costal cartilage using a method as described in Example 1. The chondrocytes capable of hypertrophication were diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension was cultured followed by a time course (4 day, 7 day, 11 day, 14 day, 18 day, 21 day) collecting supernatants of the medium.

A density of 1.25×10⁴ cells/cm² (i.e., 2.5×10⁴/well) of mouse C3H10T1/2 cells (Dainippon Sumitomo Pharmaceutical, CCL-226) were inoculated evenly in 24-well plates (Becton Dickinson). Eighteen hours after inoculation, the plates were added with 1 ml of the culture supernatant and cultured in a 5% CO₂ incubator at 37° C. After 72 hours, the alkaline phosphatase activity was measured by the method as described in Example 1. To evaluate the alkaline phosphatase activity using a relative active value, the value of alkaline phosphatase activity of a sample added with only the MEM growth medium was defined as 1. In the 4 week-old rat group, the relative active value was about 1.0 time when a culture supernatant collected after 4 days was added, about 1.3 times when a culture supernatant collected after 1 week was added, about 1.1 times when a culture supernatant collected after 2 weeks was added, and about 1.0 time when a culture supernatant collected after 3 weeks was added. In the 8 week-old rat group, the relative active value was about 1.2 times when a culture supernatant collected after 4 days was added, about 1.0 time when a culture supernatant collected after 1 week was added, about 1.0 time when a culture supernatant collected after 2 weeks was added, and about 0.9 time when a culture supernatant collected after 3 weeks was added (see Table 1, lower column, and FIG. 2). There was little difference in the alkaline phosphatase activity of the groups of the 4 and 8 week-old rats between adding the supernatant of the cell culture using MEM growth media and adding an MEM growth medium only.

(Identification of an Osteoblast)

(Staining with Alkaline Phosphatase)

Mouse C3H10T1/2 cells were inoculated in 24-well plates and hydroxyapatites (BME medium), and cultured for 18 hours. Then, the plates and hydroxyapatites were added with the culture supernatant from chondrocytes capable of hypertrophication cultured in an MEM growth medium and the cells were stained with alkaline phosphatase after 72 hours. It was confirmed that the cells were not stained with alkaline phosphatase and did not have the activity (see FIG. 3A, lower column, and FIG. 3D).

TABLE 1
(alkaline phosphatase activity in the case of addition
of the culture supernatant from chondrocytes capable
of hypertrophication cultured in MEM differentiation
agent producing medium or MEM growth medium)

| | | | | |
|---|---|---|---|---|
| 0 day | 4 day | 1 week | 2 weeks | 3 weeks |

MEM differentiation agent producing medium (mean value)

| | | | | | | |
|---|---|---|---|---|---|---|
| 4 weeks | relative value | 1 | 4.1 | 5.1 | 5.4 | 4.9 |
| old | absolute value | | 0.077 | 0.098 | 0.103 | 0.095 |
| | (addition of | | | | | |
| | supernatant) | | | | | |
| | absolute value | 0.023 | 0.023 | 0.024 | 0.023 | 0.021 |
| | (addition of | | | | | |
| | medium only) | | | | | |
| 8 weeks | relative value | 1 | 2.9 | 3.1 | 3.8 | 4.2 |
| old | absolute value | | 0.065 | 0.066 | 0.077 | 0.079 |
| | (addition of | | | | | |
| | supernatant) | | | | | |
| | absolute value | 0.021 | 0.021 | 0.021 | 0.019 | 0.019 |
| | (addition of | | | | | |
| | medium only) | | | | | |

MEM growth medium (mean value)

| | | | | | | |
|---|---|---|---|---|---|---|
| 4 weeks | relative value | 1 | 1.0 | 1.3 | 1.1 | 1.0 |
| old | absolute value | | 0.020 | 0.023 | 0.027 | 0.024 |
| | (addition of | | | | | |
| | supernatant) | | | | | |
| | absolute value | 0.022 | 0.022 | 0.020 | 0.024 | 0.024 |
| | (addition of | | | | | |
| | medium only) | | | | | |
| 8 weeks | relative value | 1 | 1.2 | 1.0 | 1.0 | 0.9 |
| old | absolute value | | 0.023 | 0.021 | 0.019 | 0.017 |
| | (addition of | | | | | |
| | supernatant) | | | | | |
| | absolute value | 0.020 | 0.020 | 0.021 | 0.019 | 0.019 |
| | (addition of | | | | | |
| | medium only) | | | | | |

4 weeks old: five experiments were performed, and 3 trials were carried out per experiment.

8 weeks old: three experiments were performed. Two trials in the first experiment, two trials in the second experiment, and one trial in the third experiment were performed.

Using the method as described in Example 1, it was confirmed that the culture supernatant from a chondrocyte capable of hypertrophication, which was obtained from the costa/costal cartilage by the above-described manipulation, cultured in an MEM growth medium did not express osteoblast markers in C3H10T1/2 cells.

### Conclusion of Example 1 and Comparative Example 1A

When chondrocytes capable of hypertrophication were cultured using an MEM differentiation agent producing medium, it was confirmed that there was the agent increasing an alkaline phosphatase activity of a mouse C3H10T1/2 cell, an undifferentiated cell, and the agent was capable of inducing the differentiation into an osteoblast. On the other hand, when chondrocytes capable of hypertrophication were cultured using an MEM growth medium, it was confirmed that there was not the agent in this culture supernatant. It was shown that a chondrocyte capable of hypertrophication produces the agent capable of inducing the differentiation of an undifferentiated cell into an osteoblast by culturing in an MEM differentiation agent producing medium. Such agent was not known hitherto. Therefore, it is believed that the existence of the agent is unexpected. Furthermore, BMP known hitherto would not have the effect of inducing the differentiation directly into osteoblasts as described in other parts.

### Comparative Example 1B

### Preparation and Detection of the Agent Produced by Culturing Resting Cartilage Cells Derived from Costal Cartilage in an MEM Differentiation Agent Producing Medium

(Preparation of Resting Cartilage Cells Derived from Costal Cartilage)

8 weeks old Male rats (Wistar) were sacrificed using chloroform. The rats' chests were shaved using a razor and their whole bodies were immersed in Hibitane (10-fold dilution) to be disinfected. The rats' chests were incised and the costal cartilage removed aseptically. The region of opaque resting cartilage was collected from the costal cartilage. The resting cartilage was sectioned and incubated in 0.25% trypsin-EDTA/D-PBS (Dulbecco's Phosphate Buffered Saline) at 37° C. for 1 hour, with stirring. The sections were then washed and collected by centrifugation (170×g for 3 min.) and followed by incubation in 0.2% Collagenase (Invitrogen)/D-PBS at 37° C. for 2.5 hours, with stirring. After washes and collection by centrifugation (170×g for 3 min.), the cells were incubated in 0.2% Dispase (Invitrogen)/(HAM+10% FBS) in a stirring flask overnight at 37° C. with stirring. Optionally, the overnight treatment with 0.2% Dispase was omitted. The following day, the resulting cell suspension was filtered and the cells washed and collected by centrifugation (170×g for 3 min.). The cells were stained with trypan blue and counted under a microscope.

The cells were evaluated as cells not stained were considered to be living cells, and those stained blue were considered to be dead cells.

(Identification of Resting Cartilage Cells without the Ability of Hypertrophication Derived from Costal Cartilage)

Using the method as described in Example 1, it was determined if chondrocytes capable of hypertrophication were present in cell suspensions obtained by diluting resting cartilage cells derived from costal cartilage. None of the hydroxyapatites was stained with alkaline phosphatase (see, FIG. 1C). With toluidine blue, the hydroxyapatites displayed blue spotted staining, showing the existence of the cells (see, FIG. 1D). Thus, it was concluded that the cells on the hydroxyapatite didn't have alkaline phosphate activity, indicating that chondrocytes without the ability of hypertrophication were present in the cell suspension used in the present Comparative Example.

By detecting the localization or expression of chondrocyte markers using the method as described in Example 1 and examining the cells morphologically, it was determined that the cells obtained were chondrocytes without the ability of hypertrophication.

(Detection of the Agent Produced by Culturing Resting Cartilage Cells Collected from the Costal Cartilage in an MEM Differentiation Agent Producing Medium)

The resting cartilage cells collected from the costal cartilage were diluted to 4×10⁴ cell/cm² in an MEM differentiation agent producing medium (Minimum Essential Medium (MEM) with a final concentration of 15% FBS (fetal bovine serum), dexamethasone 10 nM, β-glycerophosphate 10 mM, ascorbic acid 50 μg/ml, 100 U/ml penicillin, 0.1 mg/ml streptomycin and 0.25 μg/ml amphotericin B). The cell suspension was cultured and the supernatant of each medium was collected on a time course (4 day, 7 day, 11 day, 14 day, 18 day, 21 day).

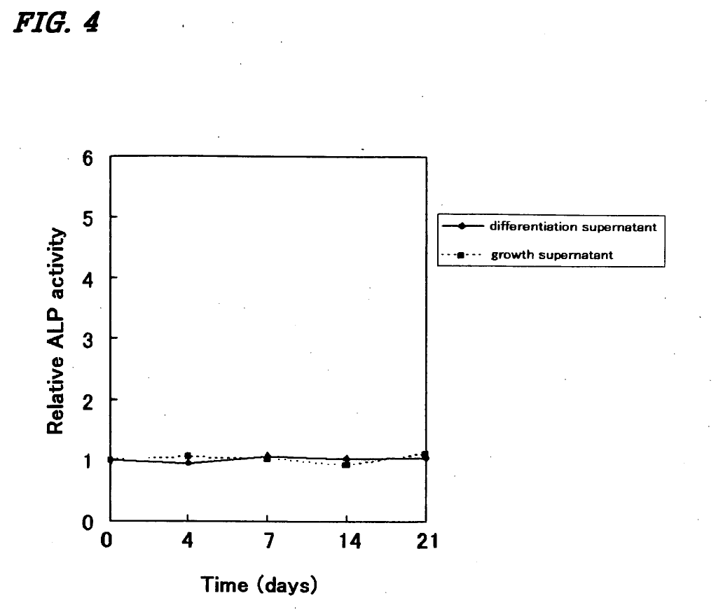

Mouse C3H10T1/2 cells (Dainippon Sumitomo Pharmaceutical, CCL-226) were inoculated evenly in 24-well plates. Eighteen hours after inoculation, 1 ml of the culture supernatant was added to the plates and cultured in a 5% CO₂ incubator at 37° C. After 72 hours, alkaline phosphatase activity was measured by the method as described in Example 1. To evaluate the alkaline phosphatase activity using a relative active value, the value of the alkaline phosphatase activity of a sample added with only the MEM growth medium was defined as 1. The relative active value was about 0.9 time when a culture supernatant collected after 4 days was added, about 1.1 times when a culture supernatant collected after 1 week was added, about 1.0 time when a culture supernatant collected after 2 weeks was added, and about 1.1 times when a culture supernatant collected after 3 weeks was added (see Table 2, upper column, and FIG. 4).

There was little difference in the alkaline phosphatase activity between adding the supernatant of the cell culture using MEM differentiation agent producing medium and adding an MEM differentiation agent producing medium only. It is confirmed that the culture supernatant of the cell culture obtained by the above-described manipulation does not express osteoblast markers in C3H10T1/2 cells.

### Comparative Example 1C

### Preparation and Detection of the Agent Produced by Culturing Resting Cartilage Cells Derived from the Costal Cartilage in an MEM Growth Medium

Resting cartilage cells were collected from the costal cartilage using the method as described in Comparative Example 1B. The resting cells were diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension was cultured and the supernatant of the medium was collected on a time course (4 day, 7 day, 11 day, 14 day, 18 day, 21 day).

Mouse C3H10T1/2 cells (Dainippon Sumitomo Pharmaceutical, CCL-226) were inoculated evenly in 24-well plates. Eighteen hours after inoculation, the plates were added with 1 ml of the culture supernatant and cultured in a 5% CO₂ incubator at 37° C. After 72 hours, alkaline phosphatase activity was measured by the method as described in Example 1. To evaluate the alkaline phosphatase activity using a relative active value, the value of alkaline phosphatase activity of a sample added with only the MEM growth medium was defined as 1. The relative active value was about 1.0 time when a culture supernatant collected after 4 days was added, about 1.0 time when a culture supernatant collected after 1 week was added, about 0.9 time when a culture supernatant collected after 2 weeks was added, and about 1.1 times when a culture supernatant collected after 3 weeks was added (see Table 2, lower column, and FIG. 4).

There was little difference in the alkaline phosphatase activity between adding the supernatant of the cell culture using MEM growth media and adding MEM growth medium only (see Table 2, lower column, and FIG. 4). It is confirmed that the culture supernatant of cell culture obtained by the above-described manipulation does not express osteoblast marker in C3H10T1/2 cell.

TABLE 2
(alkaline phosphatase activity in the case of addition
of the culture supernatant from costal cartilage-derived
resting cartilage cells cultured in MEM differentiation
agent producing medium or MEM growth medium)

| | | | | |
|---|---|---|---|---|
| 0 day | 4 day | 1 week | 2 weeks | 3 weeks |

MEM differentiation agent producing medium (mean value)

| | | | | | | |
|---|---|---|---|---|---|---|
| 8 weeks | relative value | 1 | 0.9 | 1.1 | 1.0 | 1.1 |
| old | absolute value | | 0.014 | 0.015 | 0.015 | 0.014 |
| | (addition of | | | | | |
| | supernatant) | | | | | |
| | absolute value | 0.015 | 0.015 | 0.014 | 0.014 | 0.014 |
| | (addition of | | | | | |
| | medium only) | | | | | |

MEM growth medium (mean value)

| | | | | | | |
|---|---|---|---|---|---|---|
| 8 weeks | relative value | 1 | 1.0 | 1.0 | 0.9 | 1.1 |
| old | absolute value | | 0.014 | 0.012 | 0.012 | 0.012 |
| | (addition of | | | | | |
| | supernatant) | | | | | |
| | absolute value | 0.013 | 0.013 | 0.012 | 0.011 | 0.011 |
| | (addition of | | | | | |
| | medium only) | | | | | |

8 weeks old: three experiments were performed. Three-trials in the first experiment, one trial in the second experiment, and three trials in the third experiment were performed.

### Conclusion of Comparative Example 1B and Comparative Example 1C

It was confirmed that the resting cartilage cells without the ability of hypertrophication collected from costal cartilage did not produce an agent capable of inducing the differentiation of an undifferentiated cell into an osteoblast in an MEM differentiation agent producing medium or an MEM growth medium.

### Comparative Example 1D

### Preparation and Detection of the Agent Produced by Culturing Chondrocytes Derived from Articular Cartilage in an MEM Differentiation Agent Producing Medium

(Preparation of Chondrocytes from Articular Cartilage)

8 weeks old male rats (Wistar) were sacrificed using chloroform. The rats were shaved around their knee joint region using a razor and their whole bodies were immersed in Hibitane (10-fold dilution) to be disinfected. The rats were incised at their knee joint region and the articular cartilage was removed aseptically. The articular cartilage was sectioned and stirred in 0.25% trypsin-EDTA/D-PBS at 37° C. for 1 hour. The sections were then washed and collected by centrifugation (170×g for 3 min.) and followed by stirring with 0.2% Collagenase/D-PBS at 37° C. for 2.5 hours. After washes and collection by centrifugation (170×g for 3 min.), the cells were incubated in 0.2% Dispase/(HAM+10% FBS) in stirring flask overnight at 37° C. with stirring. Optionally, the overnight treatment with 0.2% Dispase was omitted. On the following day, the cell suspension was filtered and the cells were washed and collected by centrifugation (170×g for 3 min.). The cells were stained with trypan blue and counted under a microscope.

The cells were evaluated as cells not stained were considered to be living cells, and those stained blue were considered to be dead cells.

(Identification of Chondrocytes without the Ability of Hypertrophication Derived from Articular Cartilage)

Using the method as described in Example 1, it was determined if chondrocytes capable of hypertrophication were present in the cell suspensions obtained by diluting chondrocytes derived from articular cartilage. None of the hydroxyapatites were stained with alkaline phosphatase (see FIG. 1E). With toluidine blue, the hydroxyapatites displayed blue spotted staining, showing the existence of the cells (see FIG. 1F). Thus, it was concluded that the cells on the hydroxyapatite didn't have alkaline phosphate activity, indicating that chondrocytes without the ability of hypertrophication were present in the cell suspension used in the present Comparative Example.

By detecting the localization or expression of chondrocyte markers using the method as described in Example 1 and examining the cells morphologically, it is determined that the cells obtained are chondrocytes without the ability of hypertrophication.

(Detection of the Agent Produced by Culturing Chondrocytes Collected from the Articular Cartilage in an MEM Differentiation Agent Producing Medium)

The chondrocytes collected from the articular cartilage were diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was cultured and the supernatant of each medium was collected on a time course (4 day, 7 day, 11 day, 14 day, 18 day, 21 day).

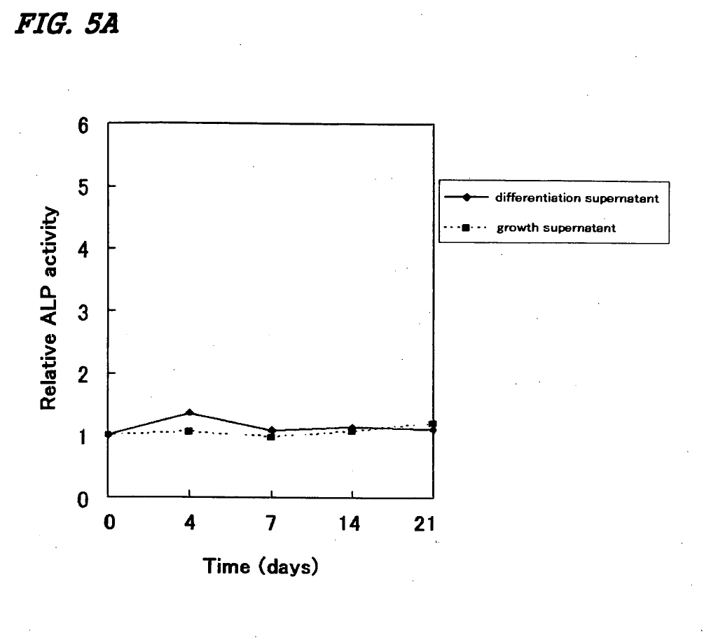

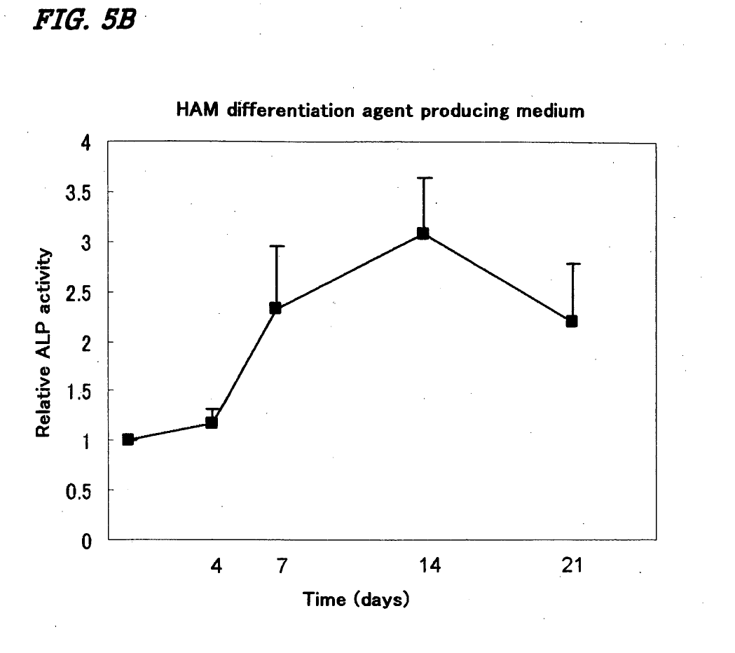

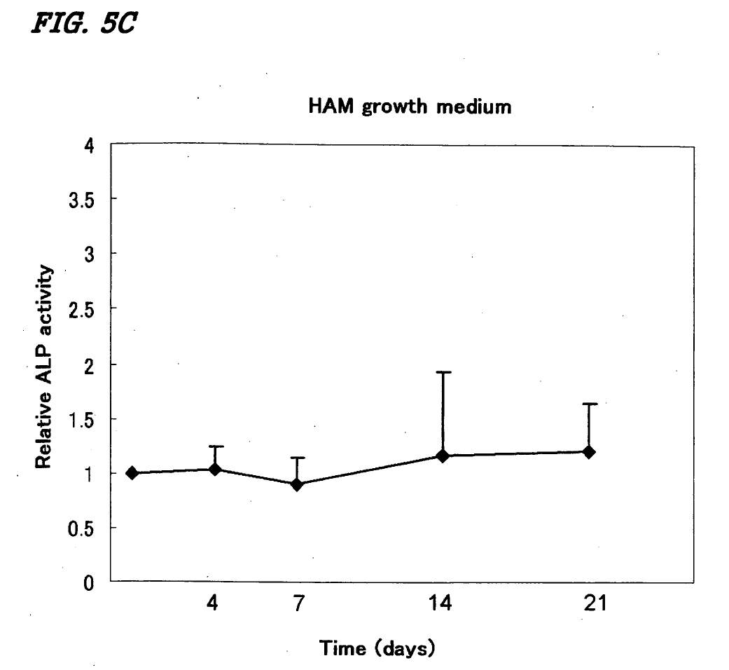

Mouse C3H10T1/2 cells (Dainippon Sumitomo Pharmaceutical, CCL-226) were inoculated evenly in 24-well plates. Eighteen hours after inoculation, the plates were added with the culture supernatant 1 ml and cultured in a 5% CO₂ incubator at 37° C. After 72 hours, alkaline phosphatase activity was measured by the method as described in Example 1. To evaluate the alkaline phosphatase activity using a relative active value, the value of the alkaline phosphatase activity of a sample added with only the MEM growth medium was defined as 1. The relative active value was about 1.4 times when a culture supernatant collected after 4 days was added, about 1.1 times when a culture supernatant collected after 1 week was added, about 1.1 times when a culture supernatant collected after 2 weeks was added, and about 1.1 times when a culture supernatant collected after 3 weeks was added (see Table 3, upper column, and FIG. 5A).

There was little difference in the alkaline phosphatase activity between adding the supernatant of the cell culture using an MEM differentiation agent producing medium and adding an MEM differentiation agent producing medium only. It is confirmed that the culture supernatant of cell culture obtained by the above-described manipulation does not express osteoblast markers in C3H10T1/2 cell.

### Comparative Example 1E

### Preparation and Detection of the Agent Produced by Culturing Chondrocytes Collected from the Articular Cartilage in an MEM Growth Medium

Chondrocytes were collected from the articular cartilage using the method as described in Comparative Example 1D. The chondrocytes were diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension was cultured and followed by collecting the supernatant of the medium on a time course (4 day, 7 day, 11 day, 14 day, 18 day, 21 day).

Mouse C3H10T1/2 cells (Dainippon Sumitomo Pharmaceutical, CCL-226) were inoculated evenly in 24-well plates. Eighteen hours after inoculation, the plates were added with 1 ml of the culture supernatant and cultured in a 5% CO₂ incubator at 37° C. After 72 hours, alkaline phosphatase activity was measured by the method as described in Example 1. To evaluate the alkaline phosphatase activity using a relative active value, the value of the alkaline phosphatase activity of a sample added with only the MEM growth medium was defined as 1. The relative active value was about 1.1 times when a culture supernatant collected after 4 days was added, about 1.0 time when a culture supernatant collected after 1 week was added, about 1.1 times when a culture supernatant collected after 2 weeks was added, and about 1.2 times when a culture supernatant collected after 3 weeks was added (see Table 3, lower column, and FIG. 5A).

There was little difference in the alkaline phosphatase activity between samples added with the supernatant of the cell culture, which were cultured with chondrocytes from articular cartilage using an MEM growth media, and those added with an MEM growth medium only (see, Table 3, lower column and FIG. 5A). It is confirmed that the culture supernatant of the cell culture obtained by the above-described manipulation does not express osteoblast markers in C3H10T1/2 cell.

TABLE 3
(Alkaline phosphatase activity in the case of adding the
culture supernatant from articular cartilage-derived chondrocytes
cultured in an MEM differentiation agent producing medium
or an MEM growth medium)
MEM differentiation agent producing medium (mean value)

| | | 0 day | 4 day | 1 week | 2 weeks | 3 weeks |
|---|---|---|---|---|---|---|
| 8 weeks | relative value | 1 | 1.4 | 1.1 | 1.1 | 1.1 |
| old | absolute value | | 0.020 | 0.019 | 0.019 | 0.020 |
| | (addition of | | | | | |
| | supernatant) | | | | | |
| | absolute value | 0.016 | 0.016 | 0.017 | 0.016 | 0.017 |
| | (addition of | | | | | |
| | medium only) | | | | | |

8 weeks old: Six experiments were performed. First experiment: one trial, Second experiment: one trial, Third experiment: three trials, Fourth experiment: two trials, Fifth experiment: one trial, Sixth experiment: one trial.

MEM growth medium (mean value)

| | | 0 day | 4 day | 1 week | 2 weeks | 3 weeks |
|---|---|---|---|---|---|---|
| 8 weeks | relative value | 1 | 1.1 | 1.0 | 1.1 | 1.2 |
| old | absolute value | | 0.019 | 0.017 | 0.017 | 0.019 |
| | (addition of | | | | | |
| | supernatant) | | | | | |
| | absolute value | 0.018 | 0.018 | 0.018 | 0.014 | 0.017 |
| | (addition of | | | | | |
| | medium only) | | | | | |

8 weeks old: Five experiments were performed. First experiment: two trial, Second experiment: two trial, Third experiment: three trials, Fourth experiment: one trials, Fifth experiment: one trial.

### Conclusion of Comparative 1D and Comparative 1E

It was confirmed that the chondrocytes without the ability of hypertrophication, which were derived from articular cartilage, did not produce an agent capable of induction to differentiate an undifferentiated cell into an osteoblast in an MEM differentiation agent producing medium or an MEM growth medium.

### Example 2

### Preparation and Detection of Cellular Function Regulating Agent Produced by Culturing a Chondrocyte Capable of Hypertrophication Derived from Sternal Cartilage in the MEM Differentiation Agent Producing Medium

(Preparation of Chondrocyte Capable of Hypertrophication from Sternal Cartilage)

Eight weeks old male rats (Wistar) are sacrificed using chloroform. The rats' chests are shaved using a razor and their whole bodies are immersed in Hibitane (10-fold dilution) to be disinfected. The rats' chests are incised and the inferior portion of sternal cartilage and processus xiphoideus are removed aseptically. The translucent growth cartilage regions are collected from the inferior portion of sternal cartilage and processus xiphoideus. The growth cartilages are sectioned and incubated in 0.25% trypsin-EDTA/Dulbecco's phosphate buffered saline (D-PBS) at 37° C. for 1 hour, with stirring. The sections are then washed and collected by centrifugation (170×g for 3 min.), followed by an incubation in 0.2% Collagenase (Invitrogen)/D-PBS at 37° C. for 2.5 hours, with stirring. After washes collection by centrifugation (170×g for 3 min.), the cells are incubated with 0.2% Dispase (Invitrogen)/(HAM+10% FBS) in a stirring flask overnight at 37° C. with stirring. The following day, the resulting cell suspensions are filtered and the cells are washed and collected by centrifugation (170×g for 3 min.). The cells are stained with trypan blue and counted under a microscope.

The cells are evaluated as cells not stained are considered to be living cells, and those stained blue are considered to be dead cells.

(Identification of a Chondrocyte Capable of Hypertrophication)

Chondrocytes capable of hypertrophication are collected and identified using the method as described in Example 1.

(Detection of an Agent Produced by Culturing a Chondrocyte Capable of Hypertrophication from Sternal Cartilage in the MEM Differentiation Agent Producing Medium)

Chondrocytes capable of hypertrophication from sternal cartilage are diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspensions are cultured and the supernatants of each medium are collected on a time course (4 day, 7 day, 11 day, 14 day, 18 day, 21 day).

Mouse C3H10T1/2 cells (Dainippon Sumitomo Pharmaceutical, CCL-226) are inoculated evenly in 24-well plates. Eighteen hours after inoculation, the plates are added with 1 ml of the culture supernatant and cultured in a 5% CO₂ incubator at 37° C. After 72 hours, alkaline phosphatase activity is measured by the method as described in Example 1.

There is an increase in the alkaline phosphatase activity of the samples adding the culture supernatant of cell culture using an MEM differentiation agent producing medium compared to those adding only an MEM differentiation agent producing medium.

(Identification of an Osteoblast)

It is confirmed that the culture supernatant obtained as described above expresses osteoblast markers in mouse C3H10T1/2 cells by, using the same manner as described in Example 1.

### Comparative Example 2

### Preparation of an Agent Produced by Culturing a Chondrocyte Capable of Hypertrophication from Sternal Cartilage in the MEM Growth Medium

Chondrocytes capable of hypertrophication are collected from the sternal cartilage using the method as described in Comparative Example 2. The chondrocytes capable of hypertrophication are diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension is cultured and followed by collecting supernatant of medium on a time course.

Mouse C3H10T1/2 cells (Dainippon Sumitomo Pharmaceutical, CCL-226) are inoculated evenly in 24-well plates. Eighteen hours after inoculation, the plates are added with 1 ml of the culture supernatant and cultured in a 5% CO₂ incubator at 37° C. After 72 hours, alkaline phosphatase activity is measured by the method as described in Example 1.

There is little difference of the alkaline phosphatase activity between samples added with the supernatant of the cell culture using an MEM growth medium and those added with an MEM growth medium only. It is confirmed that the culture supernatant of cell culture obtained by the above-described manipulation does not express osteoblast markers in C3H10T1/2 cells.

### Conclusion of Example 2 and Comparative Example 2

When chondrocytes capable of hypertrophication are cultured using an MEM differentiation agent producing medium, it is confirmed that there is the agent increasing the alkaline phosphatase activity of a mouse C3H10T1/2 cell and capable of inducing the differentiation into an osteoblast in this culture supernatant. On the other hand, when chondrocytes capable of hypertrophication are cultured using an MEM growth medium, it is confirmed that there is not the agent in this culture supernatant. It is found that a chondrocyte capable of hypertrophication produces the agent capable of inducing differentiation of an undifferentiated cell into an osteoblast by culturing in an MEM differentiation agent producing medium.

### Example 3

### Preparation and Detection of a Cellular Function Regulating Agent Produced by Culturing a Chondrocyte Capable of Hypertrophication from Costa/Costal Cartilage in the HAM Differentiation Agent Producing Medium

(Detection of an Agent Produced by Chondrocyte Capable of Hypertrophication Collected from Costa/Costal Cartilage)

The chondrocytes capable of hypertrophication obtained by Example 1 were diluted to 4×10⁴ cell/cm² in a HAM differentiation agent producing medium (HAM medium with a final concentration of 10% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was cultured and the supernatants of each medium were collected on a time course (4 day, 7 day, 11 day, 14 day, 18 day, 21 day).

Mouse C3H10T1/2 cells (Dainippon Sumitomo Pharmaceutical, CCL-226) were inoculated in 24-well plates. Eighteen hours after inoculation, the plates were added with 1 ml of the culture supernatant and cultured in a 5% CO₂ incubator at 37° C. After 72 hours, alkaline phosphatase activity was measured by the method as described in Example 1. To evaluate the alkaline phosphatase activity using a relative active value, the value of the alkaline phosphatase activity of a sample added only a HAM differentiation agent producing medium was defined as 1. The relative active value was about 1.2 times when a culture supernatant collected after 4 days was added, about 2.3 times when a culture supernatant collected after 1 week was added, about 3.1 times when a culture supernatant collected after 2 weeks was added, and about 2.2 times when a culture supernatant collected after 3 weeks was added (see Table 3-2, upper column, and FIG. 5B).

It was shown that the alkaline phosphatase (ALP) activity, which is one of the osteoblast markers, of C3H10T1/2 cells was increased by the agent capable of inducing differentiation into an osteoblast (Table 3-2 and FIG. 5B). Furthermore, the expression of alkaline phosphatase was indicated using the alkaline phosphatase staining method. As a result, it was confirmed to differentiate C3H10T1/2 cells into osteoblasts.

TABLE 3-2
(Alkaline phosphatase activity in the case of addition of
the culture supernatant from chondrocytes capable of hypertrophication
cultured in a HAM differentiation agent producing medium
or a HAM growth medium)
HAM differentiation agent producing medium (mean value)

| | 0 day | 4 day | 1 week | 2 weeks | 3 weeks |
|---|---|---|---|---|---|
| relative value | 1 | 1.2 | 2.3 | 3.1 | 2.2 |
| absolute value | 0.015 | 0.018 | 0.033 | 0.047 | 0.037 |
| (addition of | | | | | |
| supernatant) | | | | | |
| absolute value | 0.015 | | 0.014 | 0.015 | 0.017 |
| (addition of | | | | | |
| medium only) | | | | | |

Three experiments were performed. Three trials were carried out per experiment.

HAM growth medium (mean value)

| | 0 day | 4 day | 1 week | 2 weeks | 3 weeks |
|---|---|---|---|---|---|
| relative value | 1 | 1.0 | 0.9 | 1.2 | 1.2 |
| absolute value | 0.026 | 0.025 | 0.023 | 0.020 | 0.024 |
| (addition of | | | | | |
| supernatant) | | | | | |
| absolute value | 0.026 | | 0.024 | 0.021 | 0.023 |
| (addition of | | | | | |
| medium only) | | | | | |

Five experiments were performed. The First experiment: three trials; Second experiment: three trials; Third experiment: three trials; Fourth experiment: three trials; the Fifth experiment: two trials.

### Comparative Example 3A

### Preparation and Detection of the Agent Produced by Culturing a Chondrocyte Capable of Hypertrophication Derived from the Costa/Costal Cartilage in HAM Growth Medium

Chondrocytes capable of hypertrophication were collected from the costa/costal cartilage using the method as described in Example 1. The chondrocytes capable of hypertrophication were diluted to 4×10⁴ cell/cm² in HAM growth medium (HAM medium with a final concentration of 10% FBS, 100 U/ml penicillin, 0.1 mg/ml streptomycin and 0.25 μg/ml amphotericin B). The cell suspension was cultured and the supernatants of each medium were collected on a time course.

Mouse C3H10T1/2 cell (Dainippon Sumitomo Pharmaceutical, CCL-226) was inoculated in 24-well plates. Eighteen hours after inoculation, the plates were added the culture supernatant 1 ml and cultured in a 5% CO₂ incubator at 37° C. After 72 hours, alkaline phosphatase activity was measured by the method as described in Example 1. To evaluate the alkaline phosphatase activity using a relative active value, the value of the alkaline phosphatase activity of a sample added with only a HAM growth medium was defined as 1. The relative active value was about 1.0 time when a culture supernatant collected after 4 days was added, about 0.9 time when a culture supernatant collected after 1 week was added, about 1.2 times when a culture supernatant collected after 2 weeks was added, and about 1.2 times when a culture supernatant collected after 3 weeks was added (see Table 3-2, lower column, and FIG. 5C).

There was little difference of the alkaline phosphatase activity between samples added with the supernatant of the cell culture using a HAM growth media and those added with a HAM growth medium only (see Table 3-2, lower column, and FIG. 5C). It was confirmed that the culture supernatant of the cell culture obtained by the above-described manipulation did not express osteoblast markers of C3H10T1/2 cell.

### Conclusion of Example 3 and Comparative Example 3A

When chondrocytes capable of hypertrophication were cultured using a HAM differentiation agent producing medium, it was confirmed that there was the agent increasing the alkaline phosphatase activity of a mouse C3H10T1/2 cell, an undifferentiated cell, and capable of inducing differentiation into an osteoblast in this culture supernatant. On the other hand, when chondrocytes capable of hypertrophication were cultured using a HAM growth medium, it was confirmed that there was not the agent in this culture supernatant. It was found that a chondrocyte capable of hypertrophication produced the agent capable of inducing differentiation of an undifferentiated cell into an osteoblast by culturing in a HAM differentiation agent producing medium.

### Comparative Example 3B

### Preparation and Detection of the Agent Produced by Culturing Resting Cartilage Cells Derived from the Costal Cartilage in a HAM Differentiation Agent Producing Medium and a HAM Growth Medium

Resting cartilage cells are collected from the costal cartilage using the method as described in Comparative Example 1B. The resting cartilage cells are diluted to 4×10⁴ cell/cm² in a HAM differentiation agent producing medium (HAM medium with a final concentration of 10% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B) and a HAM growth medium (HAM Medium with a final concentration of 10% FBS, 100 U/ml penicillin, 0.1 mg/ml streptomycin and 0.25 μg/ml amphotericin B), respectively. The cell suspensions are cultured and the supernatants of each medium are collected on a time course.

Mouse C3H10T1/2 cells (Dainippon Sumitomo Pharmaceutical, CCL-226) are inoculated evenly in 24-well plates. Eighteen hours after inoculation, the plates are added with 1 ml of the culture supernatant and cultured. After 72 hours, alkaline phosphatase activity is measured by the method as described in Example 1.

There is little difference of the alkaline phosphatase activity between samples added with the supernatant of the cell culture using a HAM differentiation agent producing medium or a HAM growth medium, and those added with a HAM differentiation agent producing medium only or a HAM growth medium only. It is confirmed that the culture supernatant of the cell culture obtained by the above-described manipulation do not express osteoblast markers in C3H10T1/2 cells and differentiate into osteoblasts. It is confirmed the resting cartilage cells derived from costal cartilage do not produce an agent capable of inducing differentiation of an undifferentiated cell into an osteoblast in a HAM differentiation agent producing medium or a HAM growth medium.

### Conclusion of Example 1, Example 3, Comparative Examples 1A-1E 3A and 3B

According to Examples described above, the chondrocyte capable of hypertrophication produces the agent making the undifferentiated cell differentiate into an osteoblast, in spite of the type of basic medium included in the differentiation agent producing medium. The chondrocyte capable of hypertrophication does not produce the agent making the undifferentiated cell differentiate into an osteoblast in any growth medium. Furthermore, resting cartilage cells and articular cartilage cells without the ability of hypertrophication do not produce the agent making the undifferentiated cell differentiate into an osteoblast in any medium. It is suggested that the agent making the undifferentiated cell differentiate into an osteoblast is only produced by culturing a chondrocyte capable of hypertrophication in a differentiation agent producing medium. Furthermore, since the basic medium included in the medium do not affect the production of the agent of the present invention, it is believed that any medium normally used in a cell culture can be used in the present method.

### Example 4

### Preparation and Detection of Cellular Function Regulating Agent Produced by Culturing a Chondrocyte Capable of Hypertrophication Derived from Human in the MEM Differentiation Agent Producing Medium

(Detection of Chondrocyte Capable of Hypertrophication Derived from Human)

The chondrocyte capable of hypertrophication derived from human tissue (e.g., polydactyly, tumor, provided cartilaginous tissue and the like) are obtained from human tissue resource exploitation organization (domestic organization such as The Health Science Research Resources Bank; Cell Bank, RIKEN BioResource Center; Cell Bank, National Institute of Health Sciences; The Institute of Development, Aging and Cancer at Tohoku University and the like, and foreign organization such as IIAM, ATCC and the like, and cell providing company such as Osiris). The chondrocytes capable of hypertrophication obtained are diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspensions are cultured and the supernatants of each medium are collected on a time course.

The investigational human mesenchymal stem cells obtained from the above-described organizations are inoculated evenly in 24-well plates. Eighteen hours after inoculation, the plates are added with 1 ml of the culture supernatant and cultured. After 72 hours, alkaline phosphatase activity is measured by the method as described in Example 1.

It is shown that an alkaline phosphatase (ALP) activity, which is one of the osteoblast markers, of the investigational human undifferentiated cell is increased by an agent capable of inducing differentiation into an osteoblast. Furthermore, it is confirmed that the alkaline phosphatase is expressed in the alkaline phosphatase staining. As a result, it is confirmed the undifferentiated cell differentiates into an osteoblast.

### Comparative Example 4A

### Preparation and Detection of the Agent Produced by Culturing a Chondrocyte Capable of Hypertrophication Derived from Human in an MEM Growth Medium

The chondrocytes capable of hypertrophication, obtained in the same manner as Example 4, are diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension is cultured and followed by collecting the supernatant of the medium on a time course.

The investigational human undifferentiated cell is inoculated in 24-well plates. Eighteen hours after inoculation, the plates are added with 1 ml of the culture supernatant and cultured. After 72 hours, alkaline phosphatase activity is measured by the method as described in Example 1.

There is little difference of the alkaline phosphatase activity between samples added with the supernatant of the cell culture using MEM growth media and those added with an MEM growth medium only

### Conclusion of Example 4 and Comparative Example 4A

When chondrocytes capable of hypertrophication derived from human are cultured using an MEM differentiation agent producing medium, it is confirmed that the agent capable of inducing differentiation of an undifferentiated cell into an osteoblast are produced. On the other hand, when chondrocytes capable of hypertrophication derived from human are cultured using an MEM growth medium, it is confirmed that the agent capable of inducing differentiation of an undifferentiated cell into an osteoblast are not produced.

### Comparative Example 4B

### Preparation and Detection of the Agent Produced by Culturing Chondrocytes without the Ability of Hypertrophication Derived from Human in an MEM Differentiation Agent Producing Medium or an MEM Growth Medium

The chondrocytes without the ability of hypertrophication derived from human are obtained from the above-described organization. The chondrocytes are diluted to 4×10⁴ cell/cm² in an MEM differentiation agent producing medium and MEM growth medium, respectively. The cell suspensions are cultured and each supernatant of the medium is respectively collected on a time course. The investigational human undifferentiated cells are inoculated in 24-well plates. Eighteen hours after inoculation, the plates are added with 1 ml of the culture supernatant and cultured respectively. After 72 hours, alkaline phosphatase activities are measured by the method as described in Example 1.

When chondrocytes without the ability of hypertrophication derived from human are cultured in an MEM differentiation agent producing medium and an MEM growth medium, respectively, the alkaline phosphatase activities is hardly different.

It is confirmed the chondrocytes without the ability of hypertrophication derived from human do not produce an agent capable of inducing differentiation of an undifferentiated cell into an osteoblast in an MEM differentiation agent producing medium or an MEM growth medium.

### Example 5

### Preparation and Detection of a Cellular Function Regulating Agent Produced by Culturing a Chondrocyte Capable of Hypertrophication Derived from Human in the Ham Differentiation Agent Producing Medium

The chondrocytes capable of hypertrophication are obtained using the method as described in Example 4. The chondrocytes are diluted to 4×10⁴ cell/cm² in a HAM differentiation agent producing medium. The cell suspensions are cultured and each supernatant of the medium is collected on a time course. The investigational human undifferentiated cells are inoculated in 24-well plates. Eighteen hours after inoculation, the plates are added with 1 ml of the culture supernatant and cultured. After 72 hours, alkaline phosphatase activities are measured by the method as described in Example 1.

It is confirmed the chondrocytes capable of hypertrophication derived from human produce an agent capable of inducing differentiation of an undifferentiated cell into an osteoblast in a HAM differentiation agent producing medium.

### Comparative Example 5A

### Preparation and Detection of the Agent Produced by Culturing a Chondrocyte Capable of Hypertrophication Derived from Human in a HAM Growth Medium

The chondrocytes capable of hypertrophication derived from human are diluted to 4×10⁴ cell/cm² in a HAM growth medium. The cell suspensions are cultured and each supernatant of the medium is collected on a time course. The investigational human undifferentiated cells are inoculated in 24-well plates. Eighteen hours after inoculation, the plates are added with 1 ml of the culture supernatant and cultured. After 72 hours, alkaline phosphatase activities are measured by the method as described in Example 1.

It is confirmed that the chondrocytes capable of hypertrophication derived from human do not produce an agent capable of inducing differentiation of an undifferentiated cell into an osteoblast in a HAM growth medium.

### Comparative Example 5B

### Preparation and Detection of the Agent Produced by Culturing Chondrocytes without the Ability of Hypertrophication Derived from Human in a HAM Differentiation Agent Producing Medium and a HAM Growth Medium

The chondrocytes without the ability of hypertrophication, obtained in the same manner as described in Comparative Example 4B, is diluted to 4×10⁴ cell/cm² in a HAM differentiation agent producing medium and a HAM growth medium, respectively. The cell suspensions are cultured and each supernatant of the medium is respectively collected on a time course. The investigational human undifferentiated cells are inoculated in 24-well plates. Eighteen hours after inoculation, the plates is added with 1 ml of the culture supernatant and cultured, respectively. After 72 hours, alkaline phosphatase activities are measured by the method as described in Example 1.

It is confirmed that the culture supernatant obtained does not express osteoblast markers in undifferentiated cells, when the chondrocytes without the ability of hypertrophication derived from human are added culture supernatant of cell culture using a HAM differentiation agent producing medium or an HAM growth medium.

### Conclusion of Examples 4 and 5, Comparative Examples 4A-5B

According to the above-described Examples, the chondrocytes capable of hypertrophication derived from human produce the agent making the undifferentiated cell differentiate into an osteoblast, in spite of the type of basic medium included in the differentiation agent producing medium. The chondrocytes capable of hypertrophication do not produce the agent making the undifferentiated cell differentiate into an osteoblast in any growth medium. Furthermore, chondrocytes without the ability of hypertrophication do not produce the agent making the undifferentiated cell differentiate into an osteoblast in any medium. It is suggested that the agent making the undifferentiated cell differentiate into an osteoblast are only produced by culturing a chondrocyte capable of hypertrophication in a differentiation agent producing medium. Furthermore, since basic medium included in the medium does not affect the production of the agent of the present invention, it is believed that any medium normally used in a cell culture can be used in the present method.

### Example 6

### Studies on Whether the Agent Produced by a Chondrocyte Capable of Hypertrophication is Capable of Inducing Differentiation of an Undifferentiated Cell, Other than a Mouse C3H10T1/2 Cell, into an Osteoblast

Each culture supernatant was obtained by culturing chondrocytes capable of hypertrophication in an MEM differentiation agent producing medium or an MEM growth medium, using the method as described in Example 1. BALB/3T3 cells, 3T3-Swiss albino cells and NIH3T3 cells were used as the undifferentiated cells. These cells were inoculated in 24-well plates. Eighteen hours after inoculation, the plates were added with 1 ml of the culture supernatant and incubated in a 5% CO₂ incubator at 37. ° C., respectively. After 72 hours, alkaline phosphatase activities were measured by the method as described in Example 1.

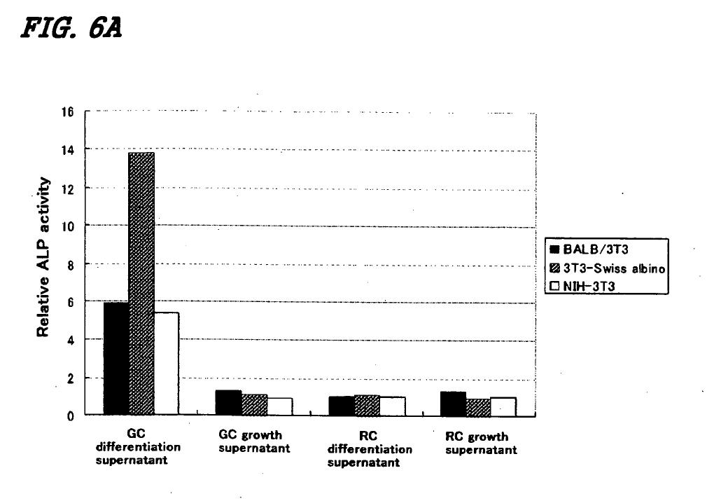

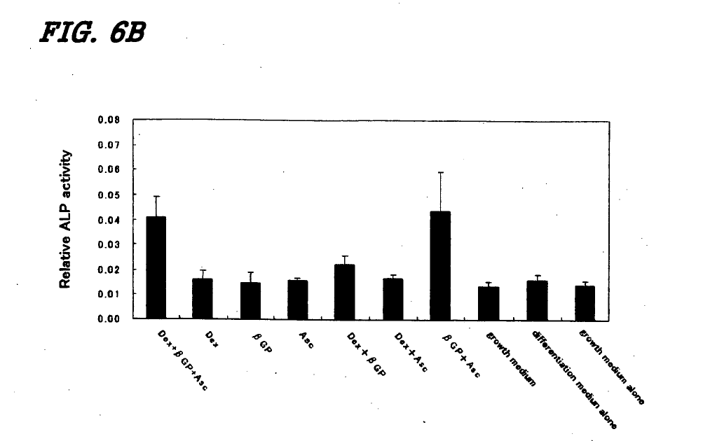

The value of alkaline phosphatase activity of a sample added with only an MEM differentiation agent producing medium was defined as 1. The value of the alkaline phosphatase activities were about 5.9 times in BALB/3T3 cells (See Table 4 left, and FIG. 6A), about 13.8 times in 3T3-Swiss albino cells (See Table 4 center and FIG. 6A), and about 5.4 times in NIH3T3 cells (See Table 4 right and FIG. 6A), when the culture supernatant from chondrocytes capable of hypertrophication cultured in an MEM differentiation agent producing medium was added.

The value of the alkaline phosphatase activity of a sample added with only the MEM growth medium was defined as 1. The value of the alkaline phosphatase activities were about 1.3 times in BALB/3T3 cells (See Table 4, left, and FIG. 6A), about 1.1 times in 3T3-Swiss albino cells (See Table 4, center, and FIG. 6A), and about 0.9 time in NIH3T3 cells (See Table 4, right, and FIG. 6A), when the culture supernatant from chondrocytes capable of hypertrophication cultured in an MEM growth medium was added.

TABLE 4
(The ability of inducing differentiation into an osteoblast from
BALB/3T3 cells, 3T3-Swiss albino cells and NIH-3T3 cells)

| | BALB/3T3 | | 3T3-Swiss albino | | NIH-3T3 | |
|---|---|---|---|---|---|---|
| | relative | absolute | relative | absolute | relative | absolute |
| | value | value | value | value | value | value |
| GC | 5.9 | 0.107 | 13.8 | 0.174 | 5.4 | 0.097 |
| differentiation | | | | | | |
| supernatant | | | | | | |
| differentiation | 1 | 0.018 | 1 | 0.013 | 1 | 0.018 |
| medium only | | | | | | |
| GC growth | 1.3 | 0.021 | 1.1 | 0.013 | 0.9 | 0.016 |
| supernatant | | | | | | |
| growth medium | 1 | 0.016 | 1 | 0.013 | 1 | 0.018 |
| only | | | | | | |

GC (4 weeks old): One experiment was performed. Three trials were performed.

GC differentiation supernatant: Culture supernatant from chondrocytes capable of hypertrophication cultured in an MEM differentiation agent producing medium

GC growth supernatant: Culture supernatant from chondrocytes capable of hypertrophication cultured in an MEM growth medium

Differentiation medium only: An MEM differentiation agent producing medium alone.

Growth medium only: An MEM growth medium alone.

When chondrocytes capable of hypertrophication are cultured using an MEM differentiation agent producing medium, it was confirmed that there is the agent increasing the alkaline phosphatase activities of 3T3-Swiss albino cells, BALB/3T3 cells and NIH3T3 cells in this culture supernatant. It was also confirmed that there are the agents increasing the alkaline phosphatase activity of these undifferentiated cells into osteoblasts. On the other hand, when chondrocytes capable of hypertrophication are cultured using an MEM growth medium, it was confirmed that there is not the agent in this culture supernatant.

### Comparative Example 6

### Studies on Whether the Components Existing in Culture Supernatant of Resting Cartilage Cells without the Ability of Hypertrophication are Capable of Inducing Differentiation of an Undifferentiated Cell, Other than a Mouse C3H10T1/2 cell, into an Osteoblast

Each culture supernatant was obtained by culturing resting cartilage cells without the ability of hypertrophication in an MEM differentiation agent producing medium and an MEM growth medium, using the method as described in Comparative Example 1B. BALB/3T3 cells, 3T3-Swiss albino cells and NIH3T3 cells were used as the undifferentiated cells. These cells were inoculated in 24-well plates. Eighteen hours after inoculation, the plates were added with 1 ml of the culture supernatant and incubated in a 5% CO₂ incubator at 37° C., respectively. After 72 hours, alkaline phosphatase activities were measured by the method as described in Example 1.

The value of the alkaline phosphatase activity of a sample added with only an MEM differentiation agent producing medium was defined as 1. The value of the alkaline phosphatase activities were about 1.0 time in BALB/3T3 cells (See Table 5, left, and FIG. 6A), about 1.1 times in 3T3-Swiss albino cells (See Table 5, center, and FIG. 6A), and about 1.0 time in NIH3T3 cells (See Table 5, right, and FIG. 6A), when the culture supernatant from resting cartilage cells without the ability of hypertrophication cultured in an MEM differentiation agent producing medium was added.

The value of the alkaline phosphatase activity of a sample added with only the MEM growth medium was defined as 1. The value of the alkaline phosphatase activities were about 1.3 times in BALB/3T3 cells (See Table 5, left, and FIG. 6A), about 0.9 time in 3T3-Swiss albino cells. (See Table 5, center, and FIG. 6A), and about 1.0 time in NIH3T3 cells (See Table 5, right, and FIG. 6A), when the culture supernatant from resting cartilage cells without the ability of hypertrophication cultured in an MEM growth medium was added.

TABLE 5
(The ability of inducing differentiation into an osteoblast from
BALB/3T3 cells, 3T3-Swiss albino cells and NIH-3T3 cells)

| | BALB/3T3 | | 3T3-Swiss albino | | NIH-3T3 | |
|---|---|---|---|---|---|---|
| | relative | absolute | relative | absolute | relative | absolute |
| | value | value | value | value | value | value |
| RC | 1.0 | 0.018 | 1.1 | 0.014 | 1.0 | 0.018 |
| differentiation | | | | | | |
| supernatant | | | | | | |
| differentiation | 1 | 0.018 | 1 | 0.013 | 1 | 0.018 |
| medium only | | | | | | |
| RC growth | 1.3 | 0.020 | 0.9 | 0.012 | 1.0 | 0.019 |
| supernatant | | | | | | |
| growth medium | 1 | 0.016 | 1 | 0.013 | 1 | 0.018 |
| only | | | | | | |

RC (8 weeks old): One experiment was performed. Three trials were performed.

RC differentiation supernatant: Culture supernatant from resting cartilage cells cultured in an MEM differentiation agent producing medium

RC growth supernatant: Culture supernatant from resting cartilage cells cultured in an MEM growth medium

Differentiation medium only: An MEM differentiation medium alone.

Growth medium only: An MEM growth medium alone.

When resting cartilage cells without the ability of hypertrophication were cultured using an MEM differentiation agent producing medium, it was confirmed that alkaline phosphatase activities differed little between those added with only an MEM differentiation agent producing medium, in 3T3-Swiss albino cells, BALB/3T3 cells or NIH3T3 cells. It was also confirmed that there were not the agents capable of inducing differentiation of these undifferentiated cells into osteoblasts. It was also confirmed that there was not the agent in these culture supernatants, when resting cartilage cells without the ability of hypertrophication were cultured using an MEM growth medium.

### Example 7

### Preparation and Detection of a Cellular Function Regulating Agent Produced by Culturing a Chondrocyte Capable of Hypertrophication from Costal Cartilage in the Medium Including the Conventional Osteoblast Differentiation Components

Chondrocytes capable of hypertrophication from costal cartilage were obtained by the method as described in Example 1. The chondrocytes were diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B), and were further added with dexamethasone, β-glycerophosphate, ascorbic acid or a combination thereof as the conventional osteoblast differentiation components. These cells were then cultured and each supernatant of the medium was collected on a time course.

| | Concentration of each osteoblast |
|---|---|
| Component added | differentiation component added |
| Dex + βGP + Asc | Dex 10 nM, βGP 10 mM, Asc 50 μg/ml |
| Dex | Dex 10 nM |
| βGP | βGP 10 mM |
| Asc | Asc 50 μg/ml |
| Dex + βGP | Dex 10 nM, βGP 10 mM |
| Dex + Asc | Dex 10 nM, Asc 50 μg/ml |
| βGP + Asc | βGP 10 mM, Asc 50 μg/ml |
| Growth medium | no osteoblast differentiation component |

Dex: dexamethasone
βGP: β-glycerophosphate
Asc: ascorbic acid

The alkaline phosphatase activities were measured, when mouse C3H10T1/2 cells (1.25×10⁴ cell/cm²) were added with 1 ml of each culture supernatant and incubated in a 5% CO₂ incubator at 37° C. The method of measuring alkaline phosphatase activity was the same as in Example 1. As shown following table and FIG. 6B, the resulting alkaline phosphatase activity was 0.041 in a medium added with an MEM differentiation agent producing medium (Dex+βGP+Asc), and 0.044 in a medium added with an MEM growth medium containing βGP and Asc (βGP+Asc). Furthermore, in a medium added with a growth medium containing individual conventional osteoblast-differentiation component, the alkaline phosphatase activity was 0.016 in the case of Dex alone, 0.015 in the case of BGP alone, and 0.016 in the case of Asc. The alkaline phosphatase activity was 0.022 in the case of a growth medium containing Dex and BGP (Dex+BGP), 0.017 in the case of the Dex and Asc (Dex+Asc). As a control, the supernatant of the medium cultured with chondrocytes capable of hypertrophication in a growth medium only was used. As a result, the alkaline phosphatase activity was 0.014, when mouse C3H10T1/2 cells were added with the medium. The alkaline phosphatase activities were 0.016 and 0.014, when C3H10T1/2 cells were added with an MEM differentiation agent producing medium only and an MEM growth medium only, respectively.

(The effect of the conventional osteoblast differentiation components on the production of the agent capable of inducing differentiation of an osteoblast)

| | mean | SD |
|---|---|---|
| Dex + βGP + Asc | 0.041 | 0.008 |
| Dex | 0.016 | 0.004 |
| βGP | 0.015 | 0.004 |
| Asc | 0.016 | 0.001 |
| Dex + βGP | 0.022 | 0.004 |
| Dex + Asc | 0.017 | 0.002 |
| βGP + Asc | 0.044 | 0.016 |
| Growth medium | 0.014 | 0.002 |
| Differentiation medium only | 0.016 | 0.002 |
| Growth medium only | 0.014 | 0.001 |

Dex: dexamethasone
βGP: β-glycerophosphate
Asc: ascorbic acid

Differentiation medium only: An MEM differentiation medium alone (i.e., a medium which was not cultured for chondrocytes)

Growth medium only: An MEM growth medium alone (i.e., a medium which was not cultured for chondrocytes)

When an MEM growth medium cultured with chondrocytes capable of hypertrophication was added with each conventional osteoblast differentiation component alone, the agent capable of inducing differentiation of undifferentiated cells into osteoblasts was not produced. When β-glycerophosphate and ascorbic acid were added, the agent capable of inducing the differentiation of undifferentiated cells into osteoblasts was produced. It was confirmed that the production of the agent capable of inducing the differentiation of undifferentiated cells into osteoblast was enhanced, when all of dexamethasone, β-glycerophosphate and ascorbic acid were added (i.e., an MEM differentiation agent producing medium).

### Example 8

### Study on the Agent Included in a Culture Supernatant Obtained by Culturing a Chondrocyte Capable of Hypertrophication in an MEM Differentiation Agent Producing Medium

Using the method as described in Example 1, the chondrocytes capable of hypertrophication were cultured in an MEM differentiation agent producing medium, and the supernatants were collected on a time course ranging from 4 day to 3 weeks. The supernatants were placed in a centrifugal filter, subjected to centrifugal ultrafiltration at 4,000×g, 4° C. for 30 minutes under conditions suitable for the separation of a macromolecular weight fraction and a low molecular weight below 50,000 fraction, to separate the supernatants containing a macromolecular weight of 50,000 or more fraction and a low molecular weight fraction. The mouse C3H10T1/2 cells (in the BME medium) were then inoculated in 24-well plates (1.25×10⁴ cell/cm²) and hydroxyapatites (1×10⁶ cells/ml). Eighteen hours after inoculation, the plates and hydroxyapatites were added with 1 ml of the fraction of each culture supernatant and cultured in a 5% CO₂ incubator at 37° C., respectively. After 72 hours, alkaline phosphatase activities were measured by the method as described in Example 1.

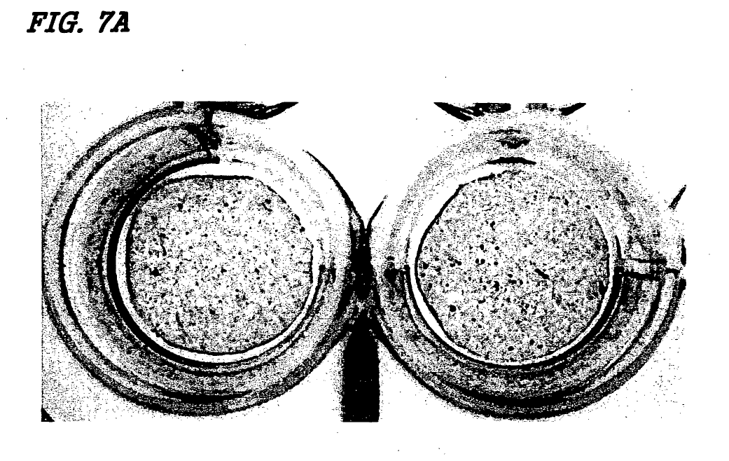

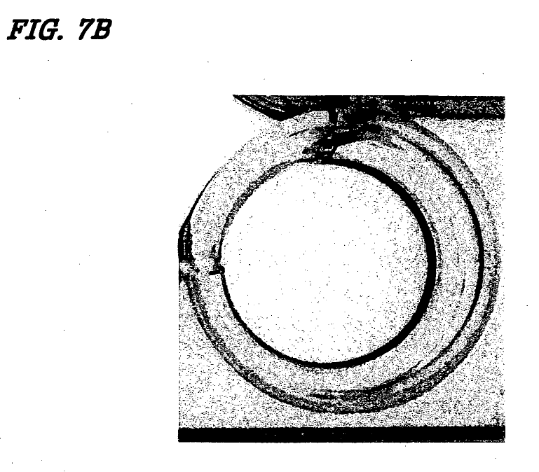

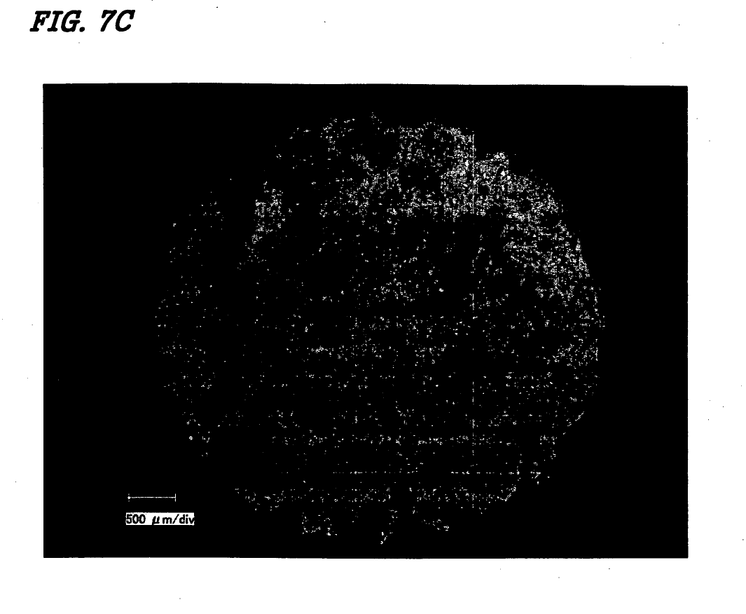

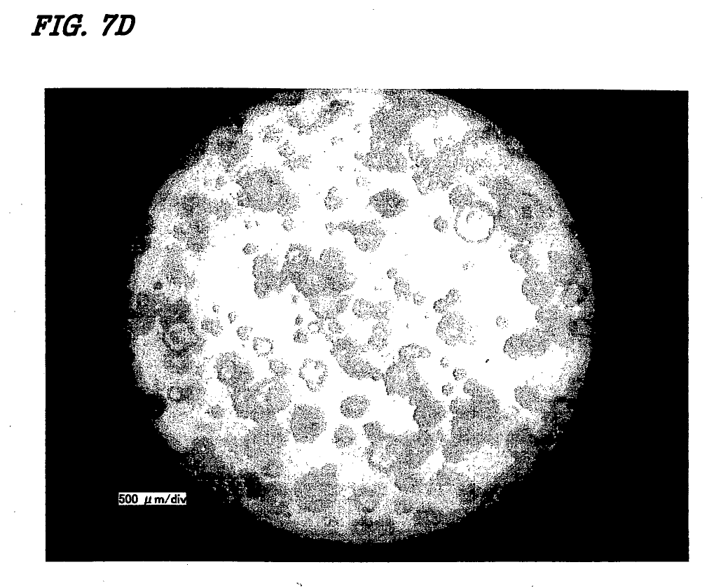

When the fraction of the supernatants thereof having a molecular weight over 50,000 was added, mouse C3H10T1/2 cells inoculated in 24-well plates and hydroxyapatites were stained red (See FIGS. 7A and 7B). It was indicated that the agent capable of increasing the alkaline phosphatase activity was present in this fraction of the supernatants thereof having a molecular weight over 50,000. When the fraction of the supernatants thereof having a molecular weight below 50,000 was added, C3H10T1/2 cells inoculated in 24-well plates and hydroxyapatites were not stained. The alkaline phosphatase activity was not observed (See FIGS. 7C and 7D).

According to the results, it was found that the agents capable of inducing the differentiation of mouse C3H10T1/2 cells into osteoblasts existed in the fraction having a molecular weight over 50,000, which was the fraction of the culture supernatant cultured with the chondrocyte capable of hypertrophication in an MEM differentiation agent producing medium.

### Example 9

### Preparation and Detection of a Cellular Function Regulating Agent Produced by Culturing a Chondrocyte Capable of Hypertrophication from Mouse Costa/Costal Cartilage in the MEM Differentiation Agent Producing Medium

(Preparation of a Chondrocyte Capable of Hypertrophication from Mouse Costa/Costal Cartilage)

Eight weeks old mice (Balb/cA) were examined in the present Example. The mice were sacrificed using chloroform. The mice's chests were shaved using a razor and their whole bodies were immersed in Hibitane (10-fold dilution) to be disinfected. The mice's chests were incised and the costa/costal cartilages were removed aseptically. The translucent growth cartilage region was collected from the boundary region of the costa/costal cartilage. The growth cartilage was sectioned and incubated in 0.25% trypsin-EDTA/Dulbecco's phosphate buffered saline (D-PBS) at 37° C. for 1 hour, with stirring. The sections were then washed and collected by centrifugation ((170×g)×3 min.) and followed by an incubation in 0.2% Collagenase (Invitrogen)/D-PBS at 37° C. for 2.5 hours, with stirring. After collection by centrifugation ((170×g)×3 min.), the cells were incubated in 0.2% Dispase (Invitrogen)/(HAM+10% FBS) in a stirring flask overnight at 37° C. with stirring. The following day, the resulting cell suspension was filtered and the cells were washed and collected by centrifugation ((170×g) for 3 min.). The cells were stained with trypan blue and counted under a microscope.

The cells were evaluated as cells not stained were considered to be living cells, and those stained blue were considered to be dead cells.

(Identification of a Chondrocyte Capable of Hypertrophication)

Since the cells obtained in Example 9 were impaired by the enzymes used in cell separation (e.g. trypsin, collagenase, and dispase), they were cultured to recover. Chondrocytes capable of hypertrophication are identified by using localization or expression of chondrocyte markers and their morphological hypertrophy under a microscope.

(Localization or Expression of Specific Markers for Chondrocytes Capable of Hypertrophication)

A cell suspension obtained using the method as described above is treated with sodium dodecyl sulfate (SDS). The SDS-treated solution is subjected to SDS polyacrylamide gel electrophoresis. The gel is then blotted onto a transfer membrane (Western blotting), reacted with a primary antibody to a chondrocyte marker, and detected with a secondary antibody labeled with an enzyme such as peroxidase, alkaline phosphatase or glucosidase, or a fluorescent tag such as fluorescein isothiocyanate (FITC), phycoerythrin (PE), Texas Red, 7-amino-4-methylcoumarin-3-acetate (AMCA) or rhodamine.

Cell cultures obtained using the method as described above are fixed with 10% neutral formalin buffer, reacted with a primary antibody to a chondrocyte marker, and detected with a secondary antibody labeled with an enzyme such as peroxidase, alkaline phosphatase or glucosidase, or a fluorescent tag such as FITC, PE, Texas Red, AMCA or rhodamine.

The alkaline phosphatase can be detected by staining. A cell culture obtained by the above-described manipulation was fixed with 60% acetone/citric acid buffer, washed with distilled water, and soaked in the mixture of First Violet B and Naphthol AS-MX at RT in the dark for 30 minutes for reaction, and thereby stained.

(Histological Assessment of the Ability of Hypertrophication in Chondrocytes)

5×10⁵ cells in a HAM's F12 medium are centrifuged to prepare a pellet of cells. The pellet is cultured with a pre-determined period. Cell sizes before and after culturing are compared under a microscope. When a significant increase in size is observed, the cells are determined to be capable of hypertrophication.

(Results)

The cells obtained in the Example 9 express a chondrocyte marker, and are determined to be morphologically hypertrophic. This shows that the cells obtained in the Example 9 are chondrocytes capable of hypertrophication. The cells are used in the following experiments.

(Detection of the Agent Produced by a Chondrocyte Capable of Hypertrophication Collected from the Mouse Costa/Costal Cartilage)

Chondrocytes capable of hypertrophication obtained by Example 9 were diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was inoculated evenly on the dish (Becton Dickinsin) and cultured in a 5% CO₂ incubator at 37° C., followed by collecting the supernatant of the medium on a time course (4 day, 7 day, 11 day, 14 day, 18 day and 21 day).

(Studies on Whether the Culture Supernatant Collected is Capable of Inducing Differentiation of an Undifferentiated Cell into an Osteoblast)

A density of 1.25×10⁴ cells/cm² of mouse C3H10T1/2 cells (Dainippon Sumitomo Pharmaceutical, CCL-226) were inoculated evenly in 24-well plates (Becton Dickinson, 2.5×10⁴/well). Eighteen hours after inoculation, the plates were added with 1 ml of the culture supernatant and cultured in a 5% CO₂ incubator at 37° C. After 72 hours, alkaline phosphatase activities were measured by the method as described in Example 1. In the present Example, the agent was determined to be capable of increasing the value of the alkaline phosphatase activity, when the agent is capable to increase the value of the alkaline phosphatase (ALP) activity of mouse C3H10T1/2 cells by more than 1.5 times comparing that of the cells cultured in the medium with or without the agent of the present invention.

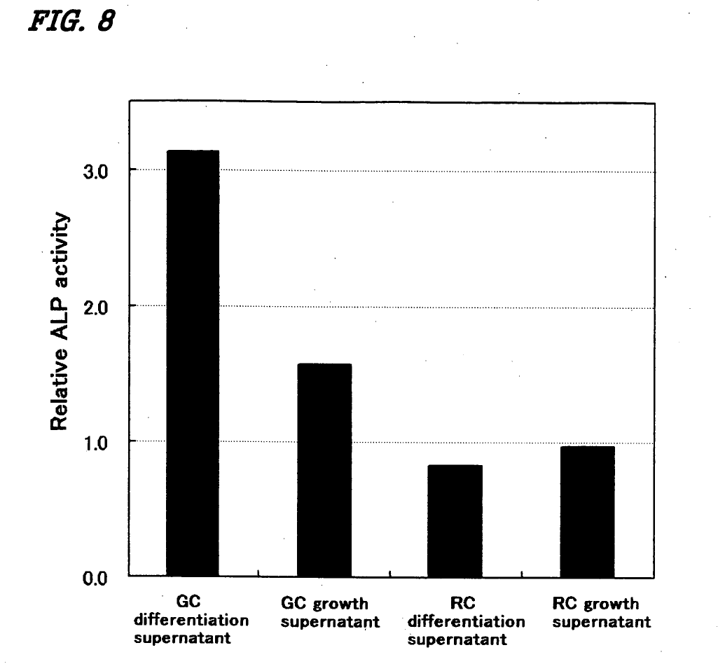

The value of the alkaline phosphatase activity of a sample added only an MEM differentiation agent producing medium was defined as 1. The value of the alkaline phosphatase activity was increased about 3.1 times (See Table 6, upper column, and FIG. 8).

(Identification of an Osteoblast)

As described above, it was shown that the alkaline phosphatase (ALP) activity, which is one of the osteoblast marker, of a C3H10T1/2 cell was increased by an agent capable of inducing differentiation into an osteoblast. Furthermore, it was also shown that C3H10T1/2 cells were stained red by adding the agent capable of inducing differentiation of osteobalasts and incubated for 72 hours in the alkaline phosphatase staining of C3H10T1/2 cells. Therefore, the expression of alkaline phosphatase is also indicated using the staining method. As a result, it was confirmed to differentiate a C3H10T1/2 cell into an osteoblast.

### Comparative Example 9A

### Preparation and Detection of the Agent Produced by Culturing a Chondrocyte Capable of Hypertrophication Derived from the Mouse Costa/Costal Cartilage in an MEM Growth Medium

Chondrocytes capable of hypertrophication were collected from the mouse costa/costal cartilage using the method as described in Example 9. The chondrocytes capable of hypertrophication were diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension was cultured and followed by collecting the supernatant of the medium on a time course (4 day, 7 day, 11 day, 14 day, 18 day, 21 day).

A density of 1.25×10⁴ cells/cm² of mouse C3H10T1/2 cells (Dainippon Sumitomo Pharmaceutical, CCL-226) were inoculated evenly in 24-well plates (Becton Dickinson, 2.5×10⁴/well). Eighteen hours after inoculation, the plates were added with 1 ml of the culture supernatant and cultured in a 5% CO₂ incubator at 37° C. After 72 hours, alkaline phosphatase activities were measured by the method as described in Example 1. The value of the alkaline phosphatase activity of a sample added with only the MEM growth medium was defined as 1. The value of the alkaline phosphatase activity was about 1.6 times (See Table 6, lower column, and FIG. 8).

There was little difference in the alkaline phosphatase activity between samples added with the supernatant of the cell culture using an MEM growth media and those added with an MEM growth medium only (see FIG. 8).

(Identification of an Osteoblast)

It was confirmed that the culture supernatant obtained as described above did not express osteoblast markers in C3H10T1/2 cells, using the method as described in Example 9.

(Comparative Example 9B: Preparation and detection of the agent produced by culturing resting cartilage cells derived from mouse costal cartilage in an MEM differentiation agent producing medium)

(Preparation of Resting Cartilage Cells Derived from Costal Cartilage)

Eight weeks old male mice (Balb/cA) were sacrificed using chloroform. The mice's chests were shaved using a razor and their whole bodies were immersed in Hibitane (10-fold dilution) to be disinfected. The mice's chests were incised and the costal cartilages-were removed aseptically. The region of the opaque resting cartilage was collected from the costal cartilage. The resting cartilage was sectioned and incubated in 0.25% trypsin-EDTA/Dulbecco's phosphate buffered saline (D-PBS) at 37° C. for 1 hour with stirring. The sections were then washed and collected by centrifugation (170×g for 3 min.), followed by an incubation in 0.2% Collagenase (Invitrogen)/D-PBS at 37° C. for 2.5 hours with stirring. After washes and collection by centrifugation (170×g for 3 min.), the cells were incubated in 0.2% Dispase (Invitrogen)/(HAM+10% FBS) in a stirring flask overnight at 37° C. with stirring. Optionally, the overnight treatment with 0.2% Dispase was omitted. The following day, the resulting cell suspension was filtered and the cells were washed and collected by centrifugation (170×g for 3 min.). The cells were stained with trypan blue and counted under a microscope.

The cells were evaluated as cells not stained were considered to be living cells, and those stained blue were considered to be dead cells.

(Identification of Chondrocytes without the Ability of Hypertrophication Derived from Costal Cartilage)

By detecting the localization or expression of chondrocyte markers using the method as described in Example 9, and examining the cells morphologically, it is confirmed that the cells obtained are chondrocytes without the ability of hypertrophication.

(Detection of the Agent Produced by Culturing Resting Cartilage Cells Collected from the Costal Cartilage in an MEM Differentiation Agent Producing Medium

Resting cartilage cells collected from the costal cartilage were diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was cultured and the supernatant of each medium was collected on a time course (4 day, 7 day, 11 day, 14 day, 18 day, 21 day).

Mouse C3H10T1/2 cells (Dainippon Sumitomo Pharmaceutical, CCL-226) were inoculated in 24-well plates. Eighteen hours after inoculation, the plates were added with 1 ml of the culture supernatant and cultured in a 5% CO₂ incubator at 37° C. After 72 hours, alkaline phosphatase activity was measured by the method as described in Example 1. The value of the alkaline phosphatase activity of a sample added with only an MEM differentiation agent producing medium was defined as 1. The value of the alkaline phosphatase activity was about 0.8 time (See Table 6, upper column, and FIG. 8).

There was little difference of the alkaline phosphatase activity between samples added with the supernatant of the cell culture using an MEM differentiation agent producing medium and added with an MEM differentiation agent producing medium only (See Table 6, upper column, and FIG. 8). It is confirmed that the culture supernatant of the cell culture obtained by the above-described manipulation does not express osteoblast markers of C3H10T1/2 cells.

### Comparative Example 9C

### Preparation and Detection of the Agent Produced by Culturing Resting Cartilage Cells Collected from the Mouse Costal Cartilage in an MEM Growth Medium)

Resting cartilage cells were collected from the costal cartilage using the method as described in Comparative Example 9B. The resting cells were diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension was cultured, followed by collecting the supernatant of the medium on a time course (4 day, 7 day, 11 day, 14 day, 18 day, 21 day).

Mouse C3H10T1/2 cells (Dainippon Sumitomo Pharmaceutical, CCL-226) were inoculated in 24-well plates. Eighteen hours after inoculation, the plates were added with 1 ml of the culture supernatant and cultured in a 5% CO₂ incubator at 37° C. After 72 hours, alkaline phosphatase activity was measured by the method as described in Example 1. The value of the alkaline phosphatase activity of a sample added with only an MEM growth medium was defined as 1. The value of alkaline phosphatase activity was about 1.0 time (See Table 6, lower column, and FIG. 8).

There was little difference of the alkaline phosphatase activity between samples added with the supernatant of the cell culture, which were cultured with resting cartilage from costal cartilage using an MEM growth medium, and those added with an MEM growth medium only (See Table 6, lower column, and FIG. 8). It was confirmed that the culture supernatant of the cell culture obtained by the above-described manipulation did not express osteoblast markers of C3H10T1/2 cells.

TABLE 6
(Comparison of the alkaline phosphatase activities added with the
culture supernatant from mouse-derived chondrocytes cultured in
an MEM differentiation agent producing medium or in an MEM growth
medium in Example 9 and Comparative Examples 9A-9C)

| | |
|---|---|
| relative value | absolute value |

MEM differentiation agent producing medium (mean value)

| | | |
|---|---|---|
| GC supernatant | 3.1 | 0.038 |
| RC supernatant | 0.8 | 0.011 |
| Differentiation medium only | 1 | 0.012 |

MEM growth medium (mean value)

| | | |
|---|---|---|
| GC supernatant | 1.6 | 0.021 |
| RC supernatant | 1.0 | 0.013 |
| Growth medium only | 1 | 0.014 |

8 weeks old: One experiment was performed. Two trials were carried out

GC supernatant: Culture supernatant from chondrocytes capable of hypertrophication cultured in the indicated medium

RC supernatant: Culture supernatant from resting cartilage cells cultured in the indicated medium

Differentiation medium only: An MEM differentiation agent producing medium alone

Growth medium only: An MEM growth medium alone

### Conclusion of Example 9 and Comparative Examples 9A-9C

When chondrocytes capable of hypertrophication, collected from mouse costa/costal cartilage, were cultured using an MEM differentiation agent producing medium, it was confirmed that there was the agent increasing the alkaline phosphatase activity of a mouse C3H10T1/2 cell and capable of inducing differentiation into an osteoblast in this culture supernatant. On the other hand, when chondrocytes capable of hypertrophication were cultured using an MEM growth medium, it was confirmed that there was not the agent in this culture supernatant. It was found that a chondrocyte capable of hypertrophication produced the agent capable of inducing differentiation of an undifferentiated cell into an osteoblast by culturing in an MEM differentiation agent producing medium.

It was confirmed that the chondrocyte without the ability of hypertrophication, derived from mouse costal cartilage, did not produce an agent capable of inducing differentiate of an undifferentiated cell into an osteoblast in an MEM differentiation agent producing medium or an MEM growth medium.

### Example 10

### Preparation and Detection of a Cellular Function Regulating Agent Produced by Culturing a Chondrocyte Capable of Hypertrophication from Rabbit Costa/Costal Cartilage in the MEM Differentiation Agent Producing Medium

(Preparation of Chondrocytes Capable of Hypertrophication from Rabbit Costa/Costal Cartilage).

Eight weeks old rabbits (Japanese White) were examined in the present Example. The rabbits were sacrificed using chloroform. The rabbits' chests were shaved using a razor and their whole bodies were immersed in Hibitane (10-fold dilution) to be disinfected. The rabbits' chests were incised and the costa/costal cartilages removed aseptically. The translucent growth cartilage region was collected from the boundary region of the costa/costal cartilage. The growth cartilage was sectioned and incubated in 0.25%-trypsin-EDTA/Dulbecco's phosphate buffered saline (D-PBS) at 37° C. for 1 hour, with stirring. The sections were then washed and collected by centrifugation ((170×g)×3 min.), followed by an incubation in 0.2% Collagenase (Invitrogen)/D-PBS at 37° C. for 2.5 hours, with stirring. After collection by centrifugation ((170×g)×3 min.), the cells were incubated in 0.2% Dispase (Invitrogen)/(HAM+10% FBS) in a stirring flask overnight at 37° C. with stirring. The following day, the resulting cell suspension was filtered and the cells were washed and collected by centrifugation ((170×g)×3 min.). The cells were stained with trypan blue and counted under a microscope.

The cells were evaluated as cells not stained were considered to be living cells, and those stained blue were considered to be dead cells.

(Identification of Chondrocytes Capable of Hypertrophication)

Since the cells obtained in Example 10 were impaired by the enzymes used in cell separation (e.g. trypsin, collagenase, and dispase), they were cultured to recover. Chondrocytes capable of hypertrophication are identified by using the localization or expression of chondrocyte markers and their morphological hypertrophy under a microscope.

(Localization or Expression of Specific Markers for Chondrocytes Capable of Hypertrophication)

A cell suspension obtained using the method as described above is treated with sodium dodecyl sulfate (SDS). The SDS-treated solution is subjected to SDS polyacrylamide gel electrophoresis. The gel is then blotted onto a transfer membrane (Western blotting), reacted with a primary antibody to a chondrocyte marker, and detected with a secondary antibody labeled with an enzyme such as peroxidase, alkaline phosphatase or glucosidase, or a fluorescent tag such as fluorescein isothiocyanate (FITC), phycoerythrin (PE), Texas Red, 7-amino-4-methylcoumarin-3-acetate (AMCA) or rhodamine.

Cell cultures obtained using the method as described above are fixed with 10% neutral formalin buffer, reacted with a primary antibody to a chondrocyte marker, and detected with a secondary antibody labeled with an enzyme such as peroxidase, alkaline phosphatase or glucosidase, or a fluorescent tag such as FITC, PE, Texas Red, AMCA or rhodamine.

The alkaline phosphatase can be detected by staining. A cell culture obtained by the above-described manipulation was fixed with 60% acetone/citric acid buffer, washed with distilled water, and soaked in the mixture of First Violet B and Naphthol AS-MX at RT in the dark for 30 minutes for reaction, and thereby stained.

(Histological Assessment of the Ability of Hypertrophication in Chondrocytes)

5×10⁵ cells in a HAM's F12 medium are centrifuged to prepare a pellet of cells. The pellet is cultured for a pre-determined period. Cell sizes before and after culturing are compared under a microscope. When a significant increase in size is observed, the cells are determined to be capable of hypertrophication.

(Results)

The cells obtained in Example 10 express a chondrocyte marker and are determined to be morphologically hypertrophic. This shows that the cells obtained in Example 10 are chondrocytes capable of hypertrophication. The cells are used in the following experiments.

(Detection of the Agent Produced by a Chondrocyte Capable of Hypertrophication Collected from the Rabbit Costa/Costal Cartilage)

Chondrocytes capable of hypertrophication obtained in Example 10 were diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was inoculated evenly on the dish (Becton Dickinsin), cultured in a 5% CO₂ incubator at 37° C. and the supernatant of each medium was collected on a time course (4 day, 7 day, 11 day, 14 day, 18 day, 21 day).

(Studies on Whether the Culture Supernatant Collected is Capable of Inducing the Differentiation of an Undifferentiated Cell into an Osteoblast)

A density of 1.25×10⁴ cells/cm² of mouse C3H10T1/2 cells (Dainippon Sumitomo Pharmaceutical, CCL-226) were inoculated evenly in 24-well plates (Becton Dickinson, 2.5×10⁴/well). Eighteen hours after inoculation, the plates were added with 1 ml of the culture supernatant and cultured in a 5% CO₂ incubator at 37° C. After 72 hours, alkaline phosphatase activity was measured by the method as described in Example 1. In the present Example, the agent was determined to be capable of increasing the value of the alkaline phosphatase activity, when the agent is capable to increase the value of the alkaline phosphatase (ALP) activity of mouse C3H10T1/2 cells by more than about 1.5 times comparing that of the cells cultured in the medium with or without the agent of the present invention.

The value of the alkaline phosphatase activity of a sample added with only the MEM differentiation agent producing medium is defined as 1. The value of alkaline phosphatase activity is increased.

(Identification of an Osteoblast)

As described above, it was shown that the alkaline phosphatase (ALP) activity, which is one of the osteoblast markers, of C3H10T1/2 cells was increased by an agent capable of inducing differentiation into an osteoblast. Furthermore, it is also shown that C3H10T1/2 cells are stained red after adding the agent capable of inducing differentiation into an osteoblast and incubating for 72 hours in the alkaline phosphatase staining of C3H10T1/2 cell. Therefore, the expression of alkaline phosphatase is indicated using the staining method. As a result, it was confirmed that C3H10T1/2 cells differentiated into osteoblasts.

### Comparative Example 10A

### Preparation and Detection of the Agent Produced by Culturing a Chondrocyte Capable of Hypertrophication Derived from the Rabbit Costa/Costal Cartilage in an MEM Growth Medium

Chondrocytes capable of hypertrophication were collected from the rabbit costa/costal cartilage using the method as described in Example 10. The chondrocytes capable of hypertrophication were diluted to 4×10⁴ cell/cm² in an MEM growth medium (Minimum Essential Medium (MEM) with a final concentration of 15% FBS, 100 U/ml penicillin, 0.1 mg/ml streptomycin and 0.25 μg/ml amphotericin B). The cell suspension was cultured and followed by collecting the supernatant of the medium on a time course (4 day, 7 day, 11 day, 14 day, 18 day, 21 day).

A density of 1.25×10⁴ cells/cm² of mouse C3H10T1/2 cells (Dainippon Sumitomo Pharmaceutical, CCL-226) were inoculated evenly in 24-well plates (Becton Dickinson, 2.5×10⁴/well). Eighteen hours after inoculation, the plates were added with 1 ml of the culture supernatant and cultured in a 5% CO₂ incubator at 37° C. After 72 hours, the alkaline phosphatase activities were measured by the method as described in Example 1.

There was little difference of the alkaline phosphatase activity between samples added with the supernatant of the cell culture using an MEM growth medium, and those added with an MEM growth medium only

(Identification of an Osteoblast)

It is confirmed that the culture supernatant obtained as described above does not express osteoblast markers in C3H10T1/2 cells, using the method as described in Example 10.

### Comparative Example 10B

### Preparation and Detection of the Agent Produced by Culturing Resting Cartilage Cells Derived from Rabbit Costal Cartilage in an MEM Differentiation Agent Producing Medium

(Preparation of Resting Cartilage Cells Derived from Costal Cartilage)

Eight weeks old male rabbits (Japanese White) were sacrificed using chloroform. The rabbits' chests were shaved using a razor and their whole bodies were immersed in Hibitane (10-fold dilution) to be disinfected. The rabbits' chests were incised and the costal cartilages were removed aseptically. The region of opaque resting cartilage was collected from the costal cartilage. The resting cartilage was sectioned and incubated in 0.25% trypsin-EDTA/Dulbecco's phosphate buffered saline (D-PBS) at 37° C. for 1 hour, with stirring. The sections were then washed and collected by centrifugation ((170×g)×3 min.), followed by an incubation in 0.2% Collagenase (Invitrogen)/D-PBS at 37° C. for 2.5 hours, with stirring. After washes and collection by centrifugation ((170×g)×3 min.), the cells were incubated in 0.2% Dispase (Invitrogen)/(HAM+10% FBS) in a stirring flask overnight at 37° C. with stirring. Optionally, the overnight treatment with 0.2% Dispase is omitted. The following day, the resulting cell suspension was filtered and the cells were washed and collected by centrifugation ((170×g)×3 min.). The cells were stained with trypan blue and counted under a microscope.

The cells were evaluated as cells not stained were considered to be living cells, and those stained blue were considered to be dead cells.

(Identification of Chondrocytes without the Ability of Hypertrophication Derived from Costal Cartilage)

By detecting the localization or expression of chondrocyte markers using the method as described in Example 10, and examining the cells morphologically, it is confirmed that the cells obtained are chondrocytes without the ability of hypertrophication.

(Detection of the Agent Produced by Culturing Resting Cartilage Cells Collected from the Costal Cartilage in an MEM Differentiation Agent Producing Medium)

Resting cartilage cells collected from the costal cartilage were diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was cultured and the supernatant of each medium was collected on a time course (4 day, 7 day, 11 day, 14 day, 18 day, 21 day).

Mouse C3H10T1/2 cells (Dainippon Sumitomo Pharmaceutical, CCL-226) were inoculated evenly in 24-well plates. Eighteen hours after inoculation, the plates were added with 1 ml of the culture supernatant and cultured in a 5% CO₂ incubator at 37° C. After 72 hours, the alkaline phosphatase activity was measured by the method as described in Example 1.

There was little difference in the alkaline phosphatase activity between samples added with the supernatant of the cell culture using an MEM differentiation agent producing medium and those added with an MEM differentiation agent producing medium only. It is confirmed that the culture supernatant of the cell culture obtained by the above-described manipulation does not express osteoblast markers of C3H10T1/2 cells.

### Comparative Example 10C

### Preparation and Detection of the Agent Produced by Culturing Resting Cartilage Cells Collected from the Costal Cartilage in an MEM Growth Medium

Resting cartilage cells were collected from the costal cartilage using the method as described in Comparative Example 10B. The resting cells were diluted to 4×10⁴ cell/cm² in MEM growth medium (Minimum Essential Medium (MEM) with a final concentration of 15% FBS, 100 U/ml penicillin, 0.1 mg/ml streptomycin and 0.25 μg/ml amphotericin B). The cell suspension was cultured and followed by collecting the supernatant of the medium on a time course (4 day, 7 day, 11 day, 14 day, 18 day, 21 day).

Mouse C3H10T1/2 cells (Dainippon Sumitomo Pharmaceutical, CCL-226) were inoculated evenly in 24-well plates. Eighteen hours after inoculation, the plates were added with 1 ml of the culture supernatant and cultured in a 5% CO₂ incubator at 37° C. After 72 hours, the alkaline phosphatase activity was measured by the method as described in Example 1.

There was little difference in the alkaline phosphatase activity between samples added with the supernatant of the cell culture, which were cultured with resting cartilage from costal cartilage using an MEM growth medium, and those added with an MEM growth medium only. It is confirmed that the culture supernatant of the cell culture obtained by the above-described manipulation does not express osteoblast markers of C3H10T1/2 cells.

### Conclusion of Example 10 and Comparative Examples 10A-10C

When chondrocytes capable of hypertrophication collected from rabbit costa/costal cartilage were cultured in an MEM differentiation agent producing medium, it was confirmed that in this culture supernatant, there was the agent increasing the alkaline phosphatase activity of an mouse C3H10T1/2 cell and capable of inducing the differentiation into an osteoblast. On the other hand, when chondrocytes capable of hypertrophication were cultured using an MEM growth medium, it was confirmed that there was not the agent in this culture supernatant. It was found that a chondrocyte capable of hypertrophication produced the agent capable of inducing the differentiation of an undifferentiated cell into an osteoblast by culturing in an MEM differentiation agent producing medium.

It was confirmed that the chondrocytes without the ability of hypertrophication, derived from rabbit costal cartilage, did not produce an agent capable of inducing differentiation of an undifferentiated cell into an osteoblast in an MEM differentiation agent producing medium or an MEM growth medium.

### Example 11

### Study on the Effect of a Medium for Culturing Undifferentiated Cells on the Differentiation of Undifferentiated Cells into Osteoblasts

Chondrocytes capable of hypertrophication, resting cartilage cells without the ability of hypertrophication and articular cartilage cells were collected using methods as described in Example 1, Comparative Example 1B and Comparative Example 1D, respectively. The cells were diluted to 4×10⁴ cell/cm² in an MEM differentiation agent producing medium and an MEM growth medium, respectively. The cell suspension was cultured in a 5% CO₂ incubator at 37° C. and each supernatant of the respective medium was collected on a time course (4 day, 7 day, 11 day, 14 day, 18 day, 21 day). The mouse C3H10T1/2 cell was used as the undifferentiated cell. A density of 1.25×10⁴ cells/cm² of these cells were inoculated in 24-well plates including a HAM medium or an MEM medium. Eighteen hours after inoculation, the plates were added with 1 ml of the culture supernatant and cultured in a 5% CO₂ incubator at 37° C., respectively. After 72 hours, the alkaline phosphatase activity was measured by the method as described in Example 1.

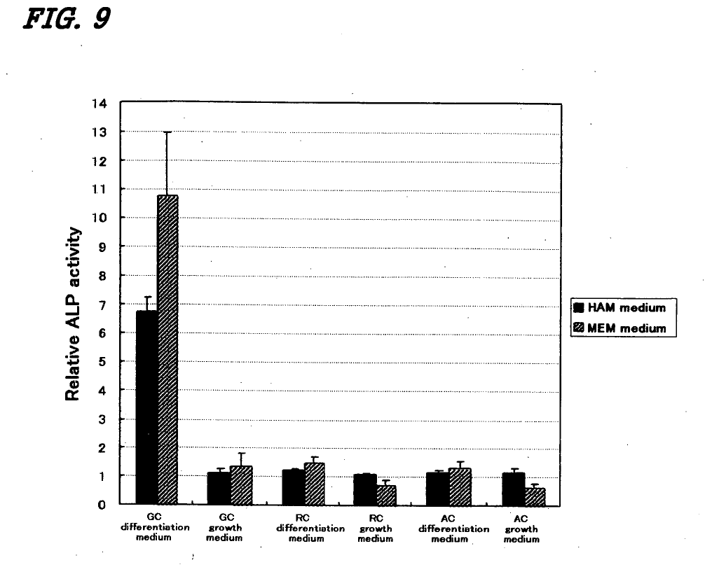

It was found that the alkaline phosphatase activity resulting from the addition of the culture supernatant from chondrocytes capable of hypertrophication cultured in an MEM differentiation agent producing medium was higher by about 6.7 times than those resulting from the addition of an MEM differentiation agent producing medium alone, when C3H10T1/2 cells were cultured in a HAM medium. The increase in alkaline phosphatase activity was not observed, when the chondrocyte capable of hypertrophication was cultured using an MEM growth medium. The increase of the alkaline phosphatase activity were not observed, when the culture supernatant used an MEM differentiation agent producing medium or an MEM growth medium, in the resting cartilage cells and chondrocytes derived from articular cartilage cells without the ability of hypertrophication, respectively (See Table 7 and FIG. 9).

It was found that the alkaline phosphatase activity resulting from the addition of the culture supernatant from chondrocytes capable of hypertrophication cultured in an MEM differentiation agent producing medium was higher by about 10.8 times than those resulting from the addition of an MEM differentiation agent producing medium alone, when C3H10T1/2 cells were cultured in an MEM medium. The increase in alkaline phosphatase activity was not observed, when the chondrocyte capable of hypertrophication was cultured using an MEM growth medium. The increases in alkaline phosphatase activity were not observed, when the culture supernatant used an MEM differentiation agent producing medium or an MEM growth medium, in the resting cartilage cells and chondrocytes derived from articular cartilage cells without the ability of hypertrophication, respectively. It was found that the basic medium used in culturing C3H1T01/2 cells did not affect the induction of differentiation of the C3H1T01/2 cells into osteoblasts (See Table 7 and FIG. 9).

TABLE 7
(the effect of a culture medium for culturing undifferentiated cells on
the differentiation of the undifferentiated cells into osteoblasts)

| | |
|---|---|
| relative value | absolute value |

HAM medium (mean value)

| | | |
|---|---|---|
| GC differentiation supernatant | 6.7 | 0.058 |
| GC growth supernatant | 1.1 | 0.010 |
| RC differentiation supernatant | 1.2 | 0.011 |
| RC growth supernatant | 1.1 | 0.010 |
| AC differentiation supernatant | 1.2 | 0.010 |
| AC growth supernatant | 1.2 | 0.011 |
| differentiation medium only | 1 | 0.009 |
| growth medium only | 1 | 0.009 |

MEM medium (mean value)

| | | |
|---|---|---|
| GC differentiation supernatant | 10.8 | 0.085 |
| GC growth supernatant | 1.3 | 0.015 |
| RC differentiation supernatant | 1.5 | 0.012 |
| RC growth supernatant | 0.7 | 0.008 |
| AC differentiation supernatant | 1.3 | 0.010 |
| AC growth supernatant | 0.6 | 0.007 |
| differentiation medium only | 1 | 0.008 |
| growth-medium only | 1 | 0.011 |

GC (4 weeks old): One experiment was performed. Three trials were performed.

RC (8 weeks old): One experiment was performed. Three trials were performed.

AC (8 weeks old): One experiment was performed. Three trials were performed.

GC differentiation supernatant: Culture supernatant from chondrocytes capable of hypertrophication cultured in an MEM differentiation agent producing medium

GC growth supernatant: Culture supernatant from chondrocytes capable of hypertrophication cultured in an MEM growth medium

RC differentiation supernatant: Culture supernatant from resting cartilage cells cultured in an MEM differentiation agent producing medium

RC growth supernatant: Culture supernatant from resting cartilage cells cultured in an MEM growth medium

AC differentiation supernatant: Culture supernatant from articular cartilage cells cultured in an MEM differentiation agent producing medium

AC growth supernatant: Culture supernatant from articular cartilage cells cultured in an MEM growth medium

Differentiation medium only: An MEM differentiation agent producing medium alone.

Growth medium only: An MEM growth medium alone.

### Example 12

### Heat Degeneration of an Agent Capable of Inducing Differentiation of an Undifferentiated Cell into an Osteoblast; the Agent is Produced by a Chondrocyte Capable of Hypertrophication)

Chondrocytes capable of hypertrophication were collected using the method as described in Example 1. The chondrocytes were diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was cultured and the supernatant of the medium was collected on a time course (4 day, 7 day, 11 day, 14 day, 18 day, 21 day). The culture supernatant was heated for 3 minutes in boiling water.

A density of 1.25×10⁴ cell/cm² of mouse C3H10T1/2 cells were cultured in the BME medium. After eighteen hours, the cells were added with 1 ml of the non-heated culture supernatant, the heated culture supernatant, and an MEM differentiation agent producing medium alone, respectively. After 72 hours, the alkaline phosphatase activities were measured by the method as described in Example 1.

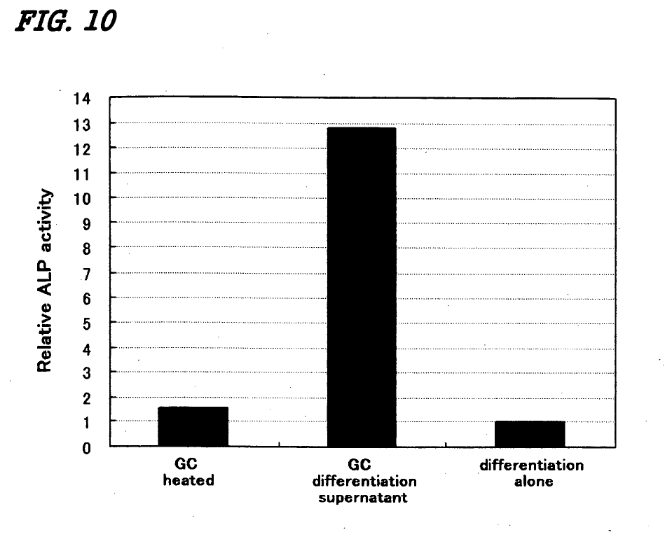

The value of the alkaline phosphatase activity of a sample added only a MEM differentiation agent producing medium was defined as 1. The value of the alkaline phosphatase activity was about 12.8 times, when non-heated culture supernatant from chondrocytes capable of hypertrophication cultured in an MEM differentiation agent producing medium was added (See Table 8 and FIG. 10). Furthermore, the value of the alkaline phosphatase activity decreased by about 1.6 times, when heated culture supernatant from chondrocytes capable of hypertrophication cultured in an MEM differentiation agent producing medium was added (See Table 8 and FIG. 10). According to the results, it was confirmed that the agent having the ability to inducing differentiation of undifferentiated cells into osteoblasts was degenerated (inactivated) by heat treatment. The agent was present in the culture supernatant from chondrocytes capable of hypertrophication cultured in an MEM differentiation agent producing medium.

TABLE 8
(Heat degerenation of an agent capable of inducing
differentiation of an undifferentiated cell into an osteoblast)
ALP activity (mean value)

| | relative value | absolute value |
|---|---|---|
| heated | 1.6 | 0.014 |
| non-treated | 12.8 | 0.111 |
| differentiation supernatant only | 1 | 0.009 |

Four weeks old: One experiment was performed. Three trials were performed.

Heated: Treated culture supernatant from chondrocytes capable of hypertrophication cultured in an MEM differentiation agent producing medium

Non-treated: Culture supernatant from chondrocytes capable of hypertrophication cultured in an MEM differentiation agent producing medium

Differentiation supernatant only: An MEM differentiation agent producing medium only.

### Example 13

### The Effect of Subcutaneous Implantation of a Composite Material Using Chondrocytes Capable of Hypertrophication Having the Ability of Producing an Agent Capable of Inducing the Differentiation of an Osteoblast, and a Biocompatible Scaffold

In the present Example, chondrocytes capable of hypertrophication are used. The chondrocytes are prepared in Examples 1-3 (rat), Examples 4-5 (human), Example 7 (rat), Example 9 (mouse), and Example 10 (rabbit). The chondrocytes capable of hypertrophication are diluted to 1×10⁶ cell/ml in an MEM differentiation agent producing medium. These cell suspensions are inoculated evenly in hydroxyapatite, PuraMatrix™ (Becton Dickinsin, catalog number 354250, BD PuraMatrix™ peptide hydrogel), collagen (gel and sponge), gelatin (sponge), agarose, alginic acid, and Matrigel™ (Becton Dickinsin), respectively, and cultured in a 5% CO₂ incubator at 37° C. for 1 week. Thereby, composite materials are prepared.

These composite materials are subcutaneously implanted into syngenic animals or immunodeficient animals. Four weeks after implantation, the syngenic animals or immunodeficient animals are sacrificed and the implanted regions are removed, fixed with 10% neutral buffered formalin, and embedded in paraffin. The sample is sectioned and stained with HE to evaluate the condition of the implanted region. Osteogenesis is observed in all of the composite materials comprising chondrocytes capable of hypertrophication, which are made to have the ability of producing the agent capable of inducing differentiation of an osteoblast by culturing in an MEM differentiation agent producing medium.

### Comparative Example A

### The Effect of Subcutaneous Implantation of a Composite Material Using Chondrocytes without the Ability of Hypertrophication and a Biocompatible Scaffold

Chondrocytes without the ability of hypertrophication are used. The chondrocytes without the ability of hypertrophication are prepared in Comparative Examples 1B, 1D and 3B (rat), Comparative Examples 4B and 5B (human), Comparative Example 9B (mouse), and Comparative Example 10B (rabbit). The chondrocytes without the ability of hypertrophication are diluted in an MEM differentiation agent producing medium and an MEM growth medium, respectively, and composite materials are prepared by using the same manner as described in Example 13. These composite materials are subcutaneously implanted into syngenic animals or immunodeficient animals. Four weeks after implantation, the syngenic animals or immunodeficient animals are sacrificed and the implanted regions are removed, fixed with 10% neutral buffered formalin, and embedded in paraffin. The sample is sectioned and stained with HE to evaluate the condition of the implanted region. Osteogenesis is not observed in any of the composite materials made of biocompatible scaffolds, when chondrocytes without the ability of hypertrophication are used.

### Comparative Example B

### The Effect of Subcutaneous Implantation of a Scaffold Alone

Hydroxyapatite, PuraMatrix™ (Becton Dickinsin, catalog number 354250, BD PuraMatrix™ peptide hydrogel), collagen (gel and sponge), gelatin (sponge), agarose, alginic acid, and Matrigel™ (Becton Dickinsin), which are scaffolds, respectively, are subcutaneously implanted into syngenic animals or immunodeficient animals, alone, using the method as described in Example 13. As a result, osteogenesis is not observed.

### Example 14

### The Effect of Subcutaneous Implantation of a Pellet of Chondrocytes Capable of Hypertrophication Having the Ability of Producing an Agent Capable of Inducing Differentiation of an Osteoblast

(Preparation of a Pellet of Chondrocytes Capable of Hypertrophication Having the Ability of Producing an Agent Capable of Inducing Differentiation of an Osteoblast)

In the present Example, chondrocytes capable of hypertrophication are used. The chondrocytes are prepared in Examples 1-3 (rat), Examples 4-5 (human), Example 7 (rat), Example 9 (mouse), and Example 10 (rabbit). MEM differentiation agent producing medium is added to these cells (5×10⁵ cells) at a final cell density of 5×10⁵ cells/0.5 ml. The cell suspensions are centrifuged (at 1000 rpm (170×g)×5 min.) to prepare pellets of chondrocytes capable of hypertrophication which are capable of inducing the differentiation of an osteoblast.

These pellets of chondrocytes capable of hypertrophication are subcutaneously implanted into syngenic animals or immunodeficient animals. Four weeks after implantation, the syngenic animals or immunodeficient animals are sacrificed and the implanted regions are removed, fixed with 10% neutral buffered formalin, and embedded in paraffin. The sample is sectioned and stained with HE to evaluate the condition of the implanted region osteogenesis is observed in all of the pellets of chondrocytes capable of hypertrophication, which have the ability of producing the agent capable of inducing differentiation of an osteoblast.

### Comparative Example A

### The Effect of Subcutaneous Implantation of a Pellet of Chondrocytes without the Ability of Hypertrophication

Chondrocytes without the ability of hypertrophication are used. The chondrocytes without the ability of hypertrophication are prepared in Comparative Examples 1B, 1D and 3B (rat), Comparative Examples 4B and 5B (human), Comparative Example 9B (mouse), and Comparative Example 10B (rabbit). MEM differentiation agent producing medium and MEM growth medium are added to these cells (5×10⁵ cells) at a final-cell density of 5×10⁵ cells/0.5 ml, respectively. The cell suspensions are centrifuged (at 1000 rpm (170×g) for 5 min.) to prepare pellets of chondrocytes without the ability of hypertrophication. The pellets of chondrocytes without the ability of hypertrophication are subcutaneously implanted into syngenic animals or immunodeficient animals. As a result, osteogenesis is not observed.

### Example 15

### The Relation Between an Agent Capable of Inducing the Differentiation of an Osteoblast Produced by Chondrocytes Capable of Hypertrophication, BMP, and TGFβ

Chondrocytes capable of hypertrophication collected from costa/costal were collected, using the method as described in Example 1. The chondrocytes were diluted to 4×10⁴ cell/cm² in an MEM differentiation agent producing medium (Minimum Essential Medium (MEM) with a final concentration of 15% FBS (fetal bovine serum), 10 nM dexamethasone, 10 mM β-glycerophosphate, 50 μg/ml ascorbic acid, 100 U/ml penicillin, 0.1 mg/ml streptomycin and 0.25 μg/ml amphotericin B). The cell suspension was cultured and followed by collecting the supernatant of the medium on a time course. Furthermore, supernatant was subjected to the following assay, and the alkaline phosphatase activity was measured.

(TGFβ Assay)

TGFβ assay was performed by a method described in Nagano, T., et al.: Effect of heat treatment on bioactivities of enamel matrix derivatives in human periodontal ligament (HPDL) cells. J. Periodont. Res., 39: 249-256, 2004. A density of 5×10⁴/well of HPDL cells were inoculated in 96-well plates and cultured for 24 hours. The culture medium was substituted with an medium including 10 nM 1a, 25-dihydroxyvitamin D3 and a test sample, and cultured for 96 hours, followed by washing with PBS. Thereafter, the alkaline phosphatase activity was measured. Specifically, the culture medium was reacted with 10 mM p-nitrophenyl phosphate as a substrate in 100 mM 2-amino-2-methyl-1,3 propanediol hydrochloric acid buffer (pH10.0) including 5 mM MgCl₂ at 37° C. for 10 minutes. The absorbance was measured at 405 nm after NaOH was added thereto.

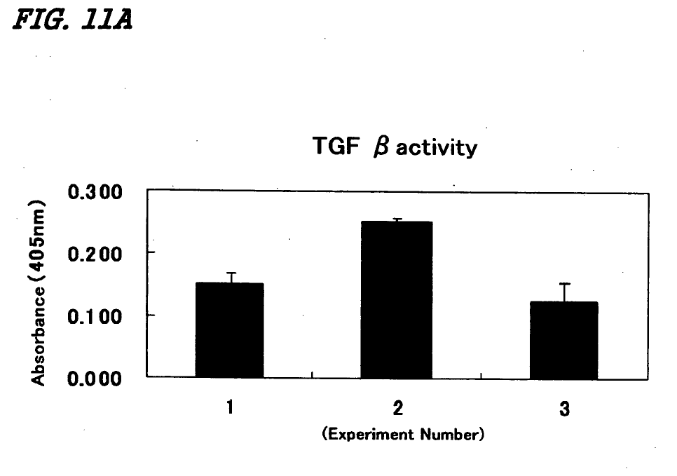

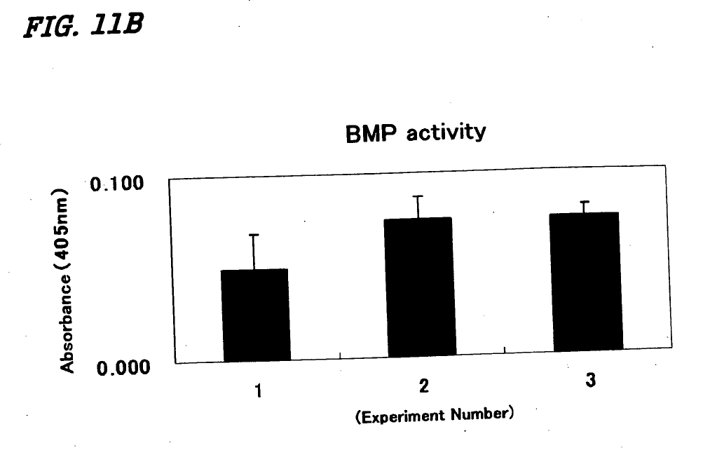

The absorbance was about 0.1515, about 0.2545, and about 0.1242, (See Table 9 and FIG. 11A) when the culture supernatant from chondrocytes capable of hypertrophication cultured in an MEM differentiation agent producing medium was added.

TABLE 9
(TGFβ activity)

| | 405 nm(OD) | SD |
|---|---|---|
| 1 | 0.1515 | 0.01818 |
| 2 | 0.2545 | 0.00303 |
| 3 | 0.1242 | 0.03030 |

(BMP Assay)

BMP assay was performed by a method described in Iwata, T., et al.: Noggin Blocks Osteoinductive Activity of Porcine Enamel Extracts. J. Dent. Res., 81: 387-391, 2002. A density of 5×10⁴/well of ST2 cells were inoculated in 96-well plates and cultured for 24 hours. The culture medium was substituted with an medium including 200 nM all-trans retinoic acid and a test sample, and cultured for 72 hours, followed by washing with PBS. Thereafter, the alkaline phosphatase activity was measured. Specifically, the culture medium was reacted with 10 mM p-nitrophenyl phosphate as a substrate in 100 mM 2-amino-2-methyl-1,3 propanediol hydrochloric acid buffer (pH10.0) including 5 mM MgCl₂ at 37° C. for 8 minutes. The absorbance was measured at 405 nm after NaOH was added thereto.

The absorbance was about 0.0500, about 0.0750, and about 0.0750, (See Table 10 and FIG. 11B) when the culture supernatant from chondrocytes capable of hypertrophication cultured in an MEM differentiation agent producing medium was added.

TABLE 10
(BMP activity)

| | 405 nm(OD) | SD |
|---|---|---|
| 1 | 0.0500 | 0.0188 |
| 2 | 0.0750 | 0.0125 |
| 3 | 0.0750 | 0.0063 |

The activity of TGFβ was observed in an MEM differentiation agent producing medium including the agent of the present invention. Therefore, It was demonstrated that TGFβ was present in this differentiation agent producing medium (See FIG. 11A). On the other hand, a slight decrease in the activity of BMP was also observed (See FIG. 11B). The BMP pathway is suppressed by the presence of TGFβ. Nevertheless, the alkaline phosphatase activity was increased in the supernatant of a differentiation agent producing medium containing TGFβ. According to the result, it is believed that the increase of the alkaline phosphatase activity was induced by an agent of the present invention, which was not BMP.

As discussed above, the present invention has been illustrated by preferred embodiments of the invention. However, the scope of the present invention should not be limited by such embodiments. It is appreciated that the present invention should be limited only by the scope of the claims. It is understood that those skilled in the art can perform equivalents of the invention according to the description of the invention or the common technical knowledge within the art. It is also understood that the contents of patents, patent application and literatures cited herein should be incorporated as references, as specifically described herein.

### INDUSTRIAL APPLICABILITY

The present invention successfully produces the agent capable of inducing the differentiation of osteoblasts from a wide rang of cells, including conventional cell lines and/or non-conventional cells, by providing a cellular function regulating agent produced by a chondrocyte capable of hypertrophication. The chondrocyte capable of hypertrophication is capable of inducing the differentiation of an undifferentiated cell into an osteoblast. Since such agent is not known, the existence of the agent itself is industrially applicable.

What is claimed is:

1. An agent obtainable by culturing a chondrocyte capable of hypertrophication in a differentiation agent producing medium, wherein the differentiation agent producing medium comprises at least one conventional osteoblast differentiation component selected from the group consisting of glucocorticoid, β-glycerophosphate and ascorbic acid.

2. The agent according to claim 1, wherein the agent is separated into a molecular weight fraction of over 50,000, wherein the culture supernatant cultured in the differentiation agent producing medium is placed in a centrifugal filter, and subjected by centrifugal ultrafiltration of 4,000×g, 4° C. for 30 minutes under conditions suitable for the separation of a macromolecular weight fraction and a low molecular weight fraction.

3. The agent according to claim 1, wherein the agent is capable of inducing the differentiation of osteoblasts from an undifferentiated cell.

4. The agent according to claim 3, wherein the undifferentiated cell is a cell which is not differentiated by glucocorticoid, β-glycerophosphate and ascorbic acid.

5. The agent according to claim 1, wherein the agent is capable of increasing the value of alkaline phosphatase (ALP) activity of a C3H10T1/2 cell which is exposed to the agent in Eagle's basal medium to be higher by more than about one times that of the cell cultured in Eagle's basal medium without the agent, wherein the alkaline phosphatase activity is determined by following steps:
A) determining two absorbances at 405 nm, wherein to one absorbance sample of 100 μl with or without the agent, 50 μl of 4 mg/ml p-nitrophenyl phosphate and 50 μl of alkali buffer (pH 10.3) 50 μl, are added respectively, reacted at 37° C. for 15 minutes, and 50 μl 1N NaOH is added to terminate the reaction, and to the other absorbance sample, a further 20 μl concentrated hydrochloric acid is added; and
B) calculating the difference in absorbance before and after addition of the concentrated hydrochloric acid, wherein the difference of absorbance is an indicator of the alkaline phosphatase activity.

6. The agent according to claim 1, wherein the agent is capable of increasing the value of alkaline phosphatase (ALP) activity of a C3H10T1/2 cell when the C3H10T1/2 cell is exposed to the agent in Eagle's basal medium, wherein the alkaline phosphatase activity is determined by following steps:
A) determining two absorbances at 405 nm, wherein to one absorbance sample of 100 μl with or without the agent, 50 μl of 4 mg/ml p-nitrophenyl phosphate and 50 μl of alkali buffer (pH 10.3) are added respectively, reacted at 37° C. for 15 minutes, and 50 μl 1N NaOH is added to terminate the reaction, and to the other absorbance sample a further 20 μl concentrated hydrochloric acid is added; and
B) calculating the difference in absorbance before and after addition of the concentrated hydrochloric acid, wherein the difference of absorbance is an indicator of the alkaline phosphatase activity.

7. The agent according to claim 1, wherein the agent is capable of enhancing expression of a specific substance for osteoblasts selected from the group consisting of type I collagen, bone proteoglycan, alkaline phosphatase, osteocalcin, matrix Gla protein, osteoglycin, osteopontin, bone sialic acid protein, osteonectin and pleiotrophin.

8. The agent according to claim 1, wherein the agent has at least one property selected from the group consisting of preventing induction of the differentiation of undifferentiated cells into osteoblasts and preventing induction of the alkaline phosphatase activity in undifferentiated cells by heating for 3 minutes in boiling water.

9. The agent according to claim 1, wherein the chondrocyte capable of hypertrophication is derived from a mammal.

10. The agent according to claim 9, wherein the mammal is a human, mouse, rat, or rabbit.

11. The agent according to claim 1, wherein the chondrocyte capable of hypertrophication is a cell sampled from the region selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture, and the cartilaginous part of a bone proliferation phase.

12. The agent according to claim 9, wherein the chondrocyte capable of hypertrophication is a cell capable of hypertrophication induced by differentiation.

13. The agent according to claim 1, wherein the chondrocyte capable of hypertrophication expresses at least one marker selected from the group consisting of type X collagen, alkaline phosphatase, osteonectin, type II collagen, cartilage proteoglycan or components thereof; hyaluronic acid, type IX collagen, type XI collagen, or chondromodulin.

14. The agent according to claim 1, wherein the ability of hypertrophication of the chondrocyte capable of hypertrophication is discriminated by morphological change.

15. The agent according to claim 1, wherein the chondrocyte capable of hypertrophication is determined to be capable of hypertrophication when a significant increase in size thereof is observed by preparing a pellet of the cells by centrifugation of HAM's F12 culture medium including 5×10⁵ cells, culturing the pellet for a pre-determined period, and comparing the size of the cells observed under a microscope before culture with the size after the culture thereof.

16. The agent according to claim 1, wherein the differentiation agent producing medium comprises at least one conventional osteoblast differentiation component selected from the group consisting of β-glycerophosphate and ascorbic acid.

17. The agent according to claim 1, wherein the differentiation agent producing medium comprises both β-glycerophosphate and ascorbic acid as the conventional osteoblast differentiation components.

18. The agent according to claim 1, wherein the differentiation agent producing medium comprises Minimum Essential Medium (MEM) or HAM Medium as the basic medium component.

19. The agent according to claim 1, wherein the differentiation agent producing medium comprises Minimum Essential Medium (MEM) as the basic component, and β-glycerophosphate and ascorbic acid as the conventional osteoblast differentiation components.

20. The agent according to claim 1, wherein the differentiation agent producing medium further comprises a serum component.

21. The agent according to claim 1, wherein the agent obtainable by culturing a chondrocyte capable of hypertrophication in a differentiation agent producing medium is obtained from the supernatant of the differentiation agent producing medium.

22. The agent according to claim 1, wherein the agent obtainable by culturing a chondrocyte capable of hypertrophication in a differentiation agent producing medium exists within the chondrocyte capable of hypertrophication.

23. A composition comprising the agent according to claim 1.

24. A composition for inducing the differentiation of osteoblasts comprising the agent according to claim 1.

25. A composition for inducing the differentiation of undifferentiated cells into osteoblasts comprising the agent according to claim 1.

26. The composition according to claim 23, wherein the composition further comprises at least one conventional osteoblast differentiation component selected from the group consisting of glucocorticoid, β-glycerophosphate and ascorbic acid.

27. The composition according to claim 23, wherein the composition further comprises at least one conventional osteoblast differentiation component selected from the group consisting of β-glycerophosphate and ascorbic acid.

28. The composition according to claim 23, wherein the composition further comprises both β-glycerophosphate and ascorbic acid as the conventional osteoblast differentiation components.

29. A method of producing a composition comprising an agent capable of inducing the differentiation of osteoblasts, wherein the method comprises culturing a chondrocyte capable of hypertrophication in a differentiation agent producing medium, wherein the differentiation agent producing medium comprises at least one conventional osteoblast differentiation component selected from the group consisting of glucocorticoid, β-glycerophosphate and ascorbic acid.

30. The method of production according to claim 29, comprising harvesting a supernatant of the differentiation agent producing medium.

31. The method of production according to claim 30, further comprising extracting from the supernatant of the differentiation agent producing medium.

32. The method of production according to claim 29, wherein the chondrocyte capable of hypertrophication is derived from a mammal.

33. The method of production according to claim 32, wherein the mammal is a human, mouse, rat, or rabbit.

34. The method of production according to claim 29, wherein the chondrocyte capable of hypertrophication is a cell sampled from the region selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture, and the cartilaginous part of a bone proliferation phase.

35. The method of production according to claim 32, wherein the chondrocyte capable of hypertrophication is a cell capable of hypertrophication induced by differentiation.

36. The method of production according to claim 29, wherein the differentiation agent producing medium comprises at least one conventional osteoblast differentiation component selected from the group consisting of β-glycerophosphate and ascorbic acid.

37. The method of production according to claim 29, wherein the differentiation agent producing medium comprises both β-glycerophosphate and ascorbic acid as the conventional osteoblast differentiation components.

38. The method of production according to claim 29, wherein the differentiation agent producing medium comprises Minimum Essential Medium (MEM) or HAM Medium as the basic medium component.

39. The method of production according to claim 29, wherein the differentiation agent producing medium comprises Minimum Essential Medium (MEM) as basic component, and β-glycerophosphate and ascorbic acid as conventional osteoblast differentiation components.

40. The method of production according to claim 29, wherein the differentiation agent producing medium further comprises a serum component.

41. The method of production according to claim 29, wherein the agent capable of inducing the differentiation is secreted into the supernatant of the differentiation agent producing medium.

42. The method of production according to claim 29, wherein the agent capable of inducing the differentiation exists within the chondrocyte capable of hypertrophication.

43. A method of producing an osteoblast by induction to differentiate an undifferentiated cell into an osteoblast, comprising the steps of:
A) inoculating the undifferentiated cell to a culture scaffold or a culture vessel; and
B) exposing the undifferentiated cell to the agent according to claim 1 by adding a solution including the agent according to claim 1 to the medium or by exchanging the medium for a medium including the agent, after the undifferentiated cell is stabilized.

44. The method according to claim 43, wherein the undifferentiated cell is derived from a mammal.

45. The method according to claim 44, wherein the mammal is a human, mouse, rat, or rabbit.

46. The method according to claim 43, wherein the chondrocyte capable of hypertrophication is a cell sampled from the region selected from the group consisting of the chondro-osseous junction of costa, epiphysial line of long bone, epiphysial line of vertebra, zone of proliferating cartilage of ossicle, perichondrium, bone primordium formed from cartilage of fetus, the callus region of a healing bone-fracture, and the cartilaginous part of a bone proliferation phase.

47. The method according to claim 43, wherein the medium culturing the undifferentiated cell is Eagle's basal medium (BME), Minimum Essential Medium (MEM), Dulbecco's Modified Eagle Medium (DMEM) or HAM medium, or a combination thereof.

48. The method according to claim 43, wherein the undifferentiated cell is selected from the group consisting of an embryonic stem cell, an embryonic germ stem cell and a tissue stem cell.

49. The method according to claim 48, wherein the tissue stem cell is selected from the group consisting of a mesenchymal stem cell, a hematopoietic stem cell, a vascular stem cell, a hepatic stem cell, a pancreatic (common) stem cell, and a neural stem cell.

50. The method according to claim 48, wherein the tissue stem cell is a mesenchymal stem cell.

51. The method according to claim 49, wherein the mesenchymal stem cell is a stem cell derived from bone marrow.

52. The method according to claim 49, wherein the mesenchymal stem cell is derived from adipose tissue, synovial tissue, muscular tissue, peripheral blood, placental tissue, menstrual blood, or cord blood.

53. The method according to claim 43, wherein the undifferentiated cell is a cell selected from the group consisting of a C3H10T1/2 cell, an ATDC5 cell, a 3T3-Swiss albino cell, a BALB/3T3 cell, and a NIH3T3 cell.

54. The method according to claim 53, wherein the undifferentiated cell is a cell selected from the group consisting of a C3H10T1/2 cell, a 3T3-Swiss albino cell, a BALB/3T3 cell, and a NIH3T 3 cell.

55. An osteoblast which is induced by contact with an agent derived from a chondrocyte capable of hypertrophication.

56. The osteoblast according to claim 55, wherein the agent is capable of increasing the value of alkaline phosphatase (ALP) activity of a C3H10T1/2 cell which is exposed to the agent in Eagle's basal medium to be higher by more than about one times that of the cell cultured in Eagle's basal medium without the agent, wherein the alkaline phosphatase activity is determined by following steps:
A) determining two absorbances at 405 nm, wherein to one absorbance sample of 100 μl with or without the agent, 50 μl of 4 mg/ml p-nitrophenyl phosphate and 50 μl of an alkali buffer (pH 10.3) are added, respectively, reacted at 37° C. for 15 minutes, and 50 μl 1N NaOH is added to terminate the reaction, and to the other absorbance sample, a further 20 μl concentrated hydrochloric acid is added; and
B) calculating the difference in absorbance before and after addition of the concentrated hydrochloric acid, wherein the difference of absorbance is an indicator of the alkaline phosphatase activity.

57. The osteoblast according to claim 55, wherein the agent is capable of increasing the value of the alkaline phosphatase (ALP) activity of a C3H10T1/2 cell when the C3H10T1/2 cell is exposed to the agent in Eagle's basal medium, wherein the alkaline phosphatase activity is determined by the following steps:
A) determining two absorbances at 405 nm, wherein to one absorbance sample of 100 μl with or without the agent, 50 μl of 4 mg/ml p-nitrophenyl phosphate and 50 μl of alkali buffer (pH 10.3) are added, respectively, reacted at 37° C. for 15 minutes, and 50 μl 1N NaOH is added to terminate the reaction, and to the other absorbance sample, a further 20 μl concentrated hydrochloric acid is added; and
B) calculating the difference in absorbance before and after addition of the concentrated hydrochloric acid, wherein the difference of absorbance is an indicator of the alkaline phosphatase activity.

58. The osteoblast according to claim 55, wherein the osteoblast is derived from an undifferentiated cell.

59. A method of producing an agent capable of inducing the differentiation of osteoblasts, wherein the method comprises culturing a chondrocyte capable of hypertrophication in a differentiation agent producing medium, wherein the differentiation agent producing medium comprises at least one conventional osteoblast differentiation component selected from the group consisting of glucocorticoid, β-glycerophosphate and ascorbic acid.

60. A composition for use in producing an agent capable of inducing the differentiation of osteoblasts, wherein the composition comprises a chondrocyte capable of hypertrophication.

61. Kit for producing an agent capable of inducing the differentiation of osteoblasts comprising:
A) a chondrocyte capable of hypertrophication; and
B) at least one conventional osteoblast differentiation component selected from the group consisting of glucocorticoid, β-glycerophosphate and ascorbic acid.

62. A composite material for producing an agent capable of inducing the differentiation of osteoblasts comprising:
A) a chondrocyte capable of hypertrophication; and
B) a scaffold.

63. The composite material according to claim 62, wherein the scaffold comprises a material selected from the group consisting of calcium phosphate, calcium carbonate, alumina, zirconia, apatite-wollastonite deposited glass, gelatin, collagen, chitin, fibrin, hyaluronic acid, extracellular matrix mixture, silk, cellulose, dextran, agarose, agar, synthetic polypeptide, polylactic acid, polyleucine, alginic acid, polyglycolic acid, polymethyl methacrylate, polycyanoacrylate, polyacrylonitrile, polyurethan, polypropylene, polyethylene, polyvinyl chloride, ethylene-vinyl acetate copolymer, nylon and a combination thereof.

64. The composite material according to claim 62, wherein the scaffold is comprised of hydroxyapatite.

65. Kit for producing an agent capable of inducing the differentiation of osteoblasts comprising:
A) the composite material according to claim 62; and
B) at least one conventional osteoblast differentiation component selected from the group consisting of glucocorticoid, β-glycerophosphate and ascorbic acid.

66. Use of a chondrocyte capable of hypertrophication in the production of an agent capable of inducing the differentiation of osteoblasts.

67. Use of a chondrocyte capable of hypertrophication and a conventional osteoblast differentiation component in production of an agent capable of inducing the differentiation of osteoblasts.

68. A composition for enhancing or inducing osteogenesis in a biological organism, wherein the composition comprises a chondrocyte capable of hypertrophication, which has potential for the inducing the differentiation of osteoblasts.

69. A composite material for enhancing or inducing osteogenesis in a biological organism, wherein the composite material comprises:
A) a chondrocyte capable of hypertrophication, which is capable of inducing the differentiation of osteoblasts; and
B) a scaffold that is biocompatible with the biological organism.

70. Kit for enhancing or inducing osteogenesis in a biological organism comprising:
A) a chondrocyte capable of hypertrophication; and
B) at least one conventional osteoblast differentiation component selected from the group consisting of glucocorticoid, β-glycerophosphate and ascorbic acid.

71. Use of:
A) a chondrocyte capable of hypertrophication, which is capable of inducing the differentiation of osteoblasts; and
B) a scaffold that is biocompatible with the biological organism,
in manufacture of an implant or a bone repairing material for enhancing or inducing osteogenesis in a biological organism.

72. A method for enhancing or inducing osteogenesis in a biological organism, comprising locating a composite material on a region in need thereof, wherein the composite material comprises a chondrocyte capable of hypertrophication, which has potential for the inducing the differentiation of osteoblasts and a scaffold that is biocompatible with the biological organism.
